(19) United States
(12) Patent Application Publication
(10) Pub. No.: US 2026/0242368 A1
(43) Pub. Date: Aug. 20, 2026

(54) GBA1 AND GCS DUAL MODULATOR

(71) Applicant: ONO PHARMACEUTICAL CO., LTD., Osaka (JP)

(72) Inventors: Takashi KONDO, Osaka (JP); Jun TAKEUCHI, Osaka (JP); Atsushi KATO, Toyama (JP)

(73) Assignee: ONO PHARMACEUTICAL CO., LTD., Osaka (JP)

(21) Appl. No.: 19/159,446

(22) PCT Filed: Feb. 28, 2024
(86) PCT No.: PCT/CN2024/078906

§ 371 Date: Aug. 25, 2025

(30) Foreign Application Priority Data
Mar. 2, 2023 (WO) PCT/CN2023/079232

(51) Int. Cl. C07D 417/12; A61K 31/55; A61K 45/06; A61P 3/00; A61P 25/28; C07D 223/08; C07D 403/12; C07D 413/12

(52) U.S. Cl. CPC C07D 417/12; A61K 31/55; A61K 45/06; A61P 3/00; A61P 25/28; C07D 223/08; C07D 403/12; C07D 413/12

(57) ABSTRACT

A compound represented by formula (I) or a pharmaceutically acceptable salt thereof is provided, which is useful as a pharmaceutical ingredient having GBA1 and GCS dual modulatory activity in the prevention and/or treatment of a disease associated with GBA1 and/or GCS, in which all symbols represent the same meaning as the symbols described in the specification. A drug is also provided, which containing, as an active ingredient, a compound having GBA1 and GCS dual modulatory activity in prevention and/or treatment of a disease associated with GBA1 and/or GCS.

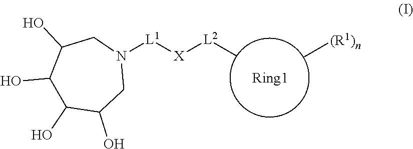

### FIELD OF THE INVENTION

The present disclosure relates to a compound having GBA1 and GCS dual modulatory activity or a pharmaceutically acceptable salt thereof. Specifically, the present disclosure relates to a medicament containing a compound represented by formula (I), or a pharmaceutically acceptable salt thereof (hereinafter, the compound is referred to as a compound of the present disclosure):

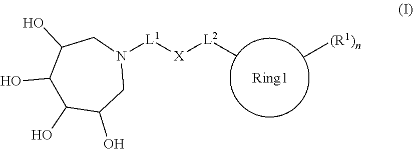

wherein all symbols represent the same meaning as the symbols described later.

### BACKGROUND ART

β-Glucocerebrosidase (GBA1) is an enzyme that hydrolyzes glucosylceramide, a glycolipid. Genetic mutations in GBA1 are known risk factors for the development of Gaucher disease, Parkinson's disease, and related synucleinopathies. In Gaucher disease, glucosylceramide accumulates in the liver, spleen, bone, and brain due to genetic mutations in GBA1, causing anemia, thrombocytopenia, hepatosplenomegaly, fracture, and serious neurological symptoms. Creation of pharmacological chaperones or gene therapy is considered to be effective as a therapeutic strategy for Gaucher disease (Non-patent document 1).

Glucosylceramide is known to be synthesized by glucosylceramide synthase (GCS), and inhibition of GCS may affect the progression of synucleinopathy by reducing glucosylceramide levels (Non-patent document 2). Therefore, compounds that regulate GBA1 and GCS may be useful in the treatment of Gaucher disease, Parkinson's disease, and related synucleinopathies.

On the other hand, Patent Document 1 states that the compound represented by the following formula (A) is a compound having ceramide glucosyltransferase inhibition, β-glucocerebrosidase inhibition, and β-glucocerebrosidase chaperone activity.

Formula (A) is as follows:

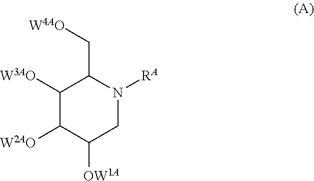

wherein R^{A} is:

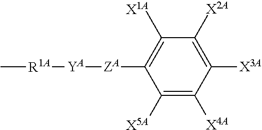

R^{IA} is a substituted or unsubstituted alkyl group;
W^{1A-4A} are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups;
X^{1A-5A} are independently selected from H, NO₂, N₃, or NH₂;
Y^{A} is absent or is a substituted or unsubstituted C1-alkyl group, other than carbonyl; and
Z^{A} is selected from a bond or NH, and the definition of groups was partially excerpted.

Patent Document 2 states that the compound represented by the following formula (B) is a compound having an increased inhibitory potency towards GCS, and/or an increased inhibitory potency towards GBA2, and/or a decreased inhibitory potency towards GBA1.

Formula (B) is as follows:

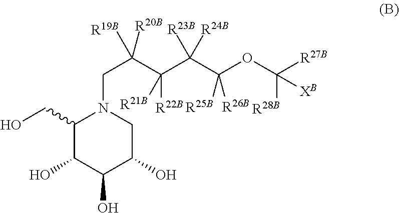

wherein each of R^{19B}-R^{26B} is independently selected from H, F, Me or Et;

R^{27B} and R^{28B} is independently selected from the group consisting of H, F, CF₃ and C1-C8 linear or branched alkyl;

X^{B} is 5-6 membered cyclic group substituted by 5-6 membered cyclic group, and the definition of groups was partially excerpted.

### PRIOR ART DOCUMENTS

### [Patent Documents]

[Patent Document 1] WO2014/179438
[Patent Document 2] WO2015/147639

### [Non-Patent Documents]

[Non-Patent Document 1] Molecular Neurodegeneration (2019) 14:36
[Non-Patent Document 2] Neurobiology of Disease 159 (2021)105507

### SUMMARY OF THE INVENTION

### Problems to be Solved by the Invention

An object of the present invention is to provide a compound having GBA1 and GCS dual modulatory activity.

### Means for Solving the Problems

As a result of intensive studies to solve the above problems, the present inventors have found that a compound represented by formula (I) described later has potent GBA1 and GCS dual modulatory activity.

That is, one aspect of the present disclosure provides the following embodiments and the like.

[A] A compound represented by formula (I) or a pharmaceutically acceptable salt thereof:

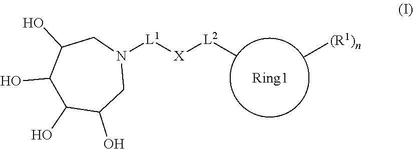

wherein
L¹ represents (1) a C4-7 alkylene, (2) a C4-7 alkenylene, or (3) a C4-7 alkynylene,
L¹ may be substituted with one to five R^{L1}s,
R^{L1} represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, or (10) a hydroxyl group,
a plurality of R^{L1}s may be the same or different,
L² represents (1) a C1-3 alkylene, (2) a C2-3 alkenylene or (3) a C2-3 alkynylene,
L² may be substituted with one to four R^{L2}s,
R^{L2} represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, or (10) a hydroxyl group,
a plurality of R^{L2}s may be the same or different,
X represents (1) a bond, (2) —O—, (3) —S—, (4) —SO—, or (5) —SO₂—,
Ring 1 represents (1) a benzene ring (2) a naphthalene ring or (3) a 5- to 10-membered heterocyclyl,
R¹ represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, (10) a C5-7 carbocyclyl, (11) a 5- to 7-membered heterocyclyl, (12) —O—(C5-7 carbocyclyl), or (13) —O-(5- to 7-membered heterocyclyl),
a plurality of R¹s may be the same or different,
the groups (2) to (13) in R¹ may be substituted with one to five R¹⁻¹s,
R¹⁻¹ represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, (10) a C1-6 alkyl sulfonyl group, (11) a cyano group, or (12) a phenyl group,
a plurality of R¹⁻¹s may be the same or different,
the symbol: [symbol]
represents that a substituent is bonded to the α-configuration, β-configuration or a mixture of α- and β-configurations at an appropriate ratio, and
n represents an integer of 0 to 5.

[B] A pharmaceutical composition comprising a compound according to the above [A] or a pharmaceutically acceptable salt thereof as an active ingredient, and a pharmaceutically acceptable carrier.

[C] The pharmaceutical composition according to the above [B], which is a GBA1 chaperone modulator.

[D] The pharmaceutical composition according to the above [B], which is a GCS inhibitor.

### Effects of the Invention

The compound of the present disclosure has GBA1 and GCS dual modulatory activity, and thus the compound of the present disclosure is useful as an agent for preventing and/or treating a disease associated with GBA1 and/or GCS.

### EMBODIMENTS FOR CARRYING OUT THE INVENTION

Hereinafter, the present disclosure will be described in detail.

In the present specification, GBA1 and GCS dual modulatory activity means both GBA1 chaperon activity and GCS inhibitory activity.

In the present specification, examples of the halogen atom include fluorine, chlorine, bromine, and iodine atoms.

In the present specification, examples of the C1-6 alkyl group include linear or branched methyl, ethyl, propyl, butyl, pentyl, hexyl groups, and isomers thereof.

In the present specification, examples of the C2-6 alkenyl group include linear or branched ethenyl, propenyl, butenyl, butadienyl, pentenyl, pentadienyl, hexenyl, hexadienyl groups, and isomers thereof.

In the present specification, examples of the C2-6 alkynyl group include linear or branched ethynyl, propynyl, butynyl, butadiynyl, pentynyl, pentadiynyl, hexynyl, hexadiynyl groups, and isomers thereof.

In the present specification, examples of the C1-3 alkylene include methylene, ethylene, propylene, and isomers thereof.

In the present specification, examples of the C2-3 alkenylene include linear or branched vinylene, propenylene, and isomers thereof.

In the present specification, examples of the C2-3 alkynylene include linear or branched ethynylene, propynylene, and isomers thereof.

In the present specification, examples of the C3-6 alkylene include linear or branched propylene, butylene, pentylene, hexylene, and isomers thereof.

In the present specification, examples of the C3-6 alkenylene include linear or branched propenylene, butenylene, pentenylene, hexenylene, and isomers thereof.

In the present specification, examples of the C3-6 alkynylene include linear or branched propynylene, butynylene, pentynylene, hexynylene, and isomers thereof.

In the present specification, examples of the C4-7 alkylene include linear or branched butylene, pentylene, hexylene, heptylene, and isomers thereof.

In the present specification, examples of the C4-7 alkenylene include linear or branched butenylene, pentenylene, hexenylene, heptenylene, and isomers thereof.

In the present specification, examples of the C4-7 alkynylene include linear or branched butynylene, pentynylene, hexynylene, heptynylene, and isomers thereof.

In the present specification, the C1-6 haloalkyl group means, for example, a linear or branched alkyl group substituted with one or more halogen atoms. Specific examples of the C1-6 haloalkyl group include a fluoromethyl group, a chloromethyl group, a bromomethyl group, an iodomethyl group, a difluoromethyl group, a trifluoromethyl group, a 1-fluoroethyl group, a 2-fluoroethyl group, a 2-chloroethyl group, a pentafluoroethyl group, a 1-fluoropropyl group, a 2-chloropropyl group, a 3-fluoropropyl group, a 3-chloropropyl group, a 4,4,4-trifluorobutyl group, a 4-bromobutyl group, a 5,5,5-trifluoropentyl group, a 6,6,6-trifluorohexyl group, and isomers thereof.

In the present specification, the C2-6 haloalkenyl group means, for example, a linear or branched alkenyl group substituted with one or more halogen atoms. Specific examples of the C2-6 haloalkenyl group include a 1-fluoroethenyl group, a 2-fluoroethenyl group, a 2-chloroethenyl group, a 1-fluoropropenyl group, a 2-chloropropenyl group, a 3-fluoropropenyl group, a 3-chloropropenyl group, a 4-bromobutenyl group, a 5,5,5-trifluoropentenyl group, a 6,6,6-trifluorohexenyl group, and isomers thereof.

In the present specification, the C2-6 haloalkynyl group means, for example, a linear or branched alkynyl group substituted with one or more halogen atoms. Specific examples of the C2-6 haloalkynyl group include a 2-fluoroethynyl group, a 2-chloroethynyl group, a 3-fluoropropynyl group, a 3-chloropropynyl group, a 4-bromobutynyl group, a 5,5,5-trifluoropentynyl group, a 6,6,6-trifluorohexynyl group, and isomers thereof.

In the present specification, examples of the C1-6 alkoxy group includes linear or branched methoxy, ethoxy, propoxy, butoxy, pentyloxy, hexyloxy groups, and isomers thereof.

In the present specification, the C1-6 haloalkoxy group means, for example, a linear or branched alkoxy group substituted with one or more halogen atoms. Specific examples of the C1-6 haloalkoxy group include a fluoromethoxy group, a chloromethoxy group, a bromomethoxy group, an iodomethoxy group, a difluoromethoxy group, a trifluoromethoxy group, a 1-fluoroethoxy group, a 2-fluoroethoxy group, a 2-chloroethoxy group, a pentafluoroethoxy group, a 1-fluoropropoxy group, a 2-fluoropropoxy group, a 2-chloropropoxy group, a 3-fluoropropoxy group, a 3-chloropropoxy group, a 4,4,4-trifluorobutoxy group, a 4-bromobutoxy group, a 5,5,5-trifluoropentyloxy group, a 6,6,6-trifluorohexyloxy group, and isomers thereof.

In the present specification, examples of the C5-7 carbocyclyl include cyclopentane, cyclohexane, cycloheptane, cyclopentene, cyclohexene, cycloheptene, cyclopentadiene, cyclohexadiene, cycloheptadiene, and benzene rings.

In the present specification, examples of the 5- to 7-membered heterocyclyl include pyrrole, imidazole, triazole, tetrazole, pyrazole, pyridine, pyrazine, pyrimidine, pyridazine, furan, pyran, thiophene, thiopyran, oxazole, isoxazole, thiazole, isothiazole, furazan, oxadiazole, oxazine, oxadiazine, thiadiazole, thiazine, thiadiazine, pyrroline, pyrrolidine, imidazoline, imidazolidine, triazoline, triazolidine, tetrazoline, tetrazolidine, pyrazoline, pyrazolidine, dihydropyridine, tetrahydropyridine, piperidine, dihydropyrazine, tetrahydropyrazine, piperazine, dihydropyrimidine, tetrahydropyrimidine, perhydropyrimidine, dihydropyridazine, tetrahydropyridazine, perhydropyridazine, dihydrofuran, tetrahydrofuran, dihydropyran, tetrahydropyran, dihydrothiophene, tetrahydrothiophene, dihydrothiopyran, tetrahydrothiopyran, dihydrooxazole, tetrahydrooxazole (oxazolidine), dihydroisoxazole, tetrahydroisoxazole (isoxazolidine), dihydrothiazole, tetrahydrothiazole (thiazolidine), dihydroisothiazole, tetrahydroisothiazole (isothiazolidine), dihydrofurazan, tetrahydrofurazan, dihydrooxadiazole, tetrahydrooxadiazole (oxadiazolidine), dihydrooxazine, tetrahydrooxadiazine, dihydrooxadiazine, tetrahydrooxadiazine, dihydrothiadiazole, tetrahydrothiadiazole (thiadiazolidine), dihydrothiazine, tetrahydrothiazine, dihydrothiadiazine, tetrahydrothiadiazine, morpholine, thiomorpholine, oxathiane, dioxolane, dioxane, dithiolane, dithiane, azepine, diazepine, oxepin, thiepin, oxazepine, oxadiazepine, thiazepine, thiadiazepine, dihydroazepine, tetrahydroazepine, perhydroazepine, dihydrodiazepine, tetrahydrodiazepine, perhydrodiazepine, dihydrooxepin, tetrahydrooxepin, perhydrooxepin, dihydrothiepin, tetrahydrothiepin, perhydrothiepin, dihydrooxazepine, tetrahydrooxazepine, perhydrooxazepine, dihydrooxadiazepine, tetrahydrooxadiazepine, perhydrooxadiazepine, dihydrothiazepine, tetrahydrothiazepine, perhydrothiazepine, dihydrothiadiazepine, tetrahydrothiadiazepine, and perhydrothiadiazepine rings.

In the present specification, examples of the 5- to 10-membered heterocyclyl include the 5- to 7-membered heterocyclyl, and indole, isoindole, indolizine, benzofuran, isobenzofuran, benzothiophene, isobenzothiophene, dithianaphthalene, indazole, quinoline, isoquinoline, quinolizine, purine, phthalazine, pteridine, naphthyridine, quinoxaline, quinazoline, cinnoline, benzoxazole, benzothiazole, benzimidazole, chromene, benzofurazan, benzothiadiazole, benzotriazole, indoline, isoindoline, dihydrobenzofuran, perhydrobenzofuran, dihydroisobenzofuran, perhydroisobenzofuran, dihydrobenzothiophene, perhydrobenzothiophene, dihydroisobenzothiophene, perhydroisobenzothiophene, dihydroindazole, perhydroindazole, dihydroquinoline, tetrahydroquinoline, perhydroquinoline, dihydroisoquinoline, tetrahydroisoquinoline, perhydroisoquinoline, dihydrophthalazine, tetrahydrophthalazine, perhydrophthalazine, dihydronaphthyridine, tetrahydronaphthyridine, perhydronaphthyridine, dihydroquinoxaline, tetrahydroquinoxaline, perhydroquinoxaline, dihydroquinazoline, tetrahydroquinazoline, perhydroquinazoline, dihydrocinnoline, tetrahydrocinnoline, perhydrocinnoline, benzoxathiane, dihydrobenzoxazine, dihydrobenzothiazine, pyrazinomorpholine, dihydrobenzoxazole, perhydrobenzoxazole, dihydrobenzothiazole, perhydrobenzothiazole, dihydrobenzimidazole, perhydrobenzimidazole, dioxaindane, benzodioxane, chroman, benzodithiolane, benzodithiane, azaspiro[4.4]nonane, oxazaspiro[4.4]nonane, dioxaspiro[4.4]nonane, azaspiro[4.5]decane, thiaspiro[4.5]decane, dithiaspiro[4.5]decane, dioxaspiro[4.5]decane, oxazaspiro[4.5]decane, azabicyclo[3.2.1]octane, oxabicyclo[3.2.1]octane, thieno[3,2-c]pyrazole, thieno[2,3-c]pyrazole, thieno[2,3-d]thiazole, thieno[2,3-d][1.2.3]triazole, dihydropyrano[3,4-d]thiazole, dihydrothieno[2,3-b]pyran, dihydrothieno[3,2-c]pyran, dihydrothieno[3,2-b]pyran, dihydrothieno[3,2-c]thiopyran, tetrahydrothieno[3,2-b]pyridine, tetrahydrothieno[3,2-c]pyridine, and thieno[3,2-c]pyridine rings.

In the present disclosure, L¹ is preferably a C4-7 alkylene group, and more preferably a hexylene group.

In the present disclosure, L² is preferably a methylene.

In the present disclosure, X is preferably —O— or —S—, and more preferably —O—.

In the present disclosure, Ring 1 is preferably a benzene ring or a 5- to 7-membered heterocyclyl, and more preferably a benzene ring, a thiazole ring, an oxazole ring or pyridazine ring, and still more preferably a ring structure selected from the group consisting of:

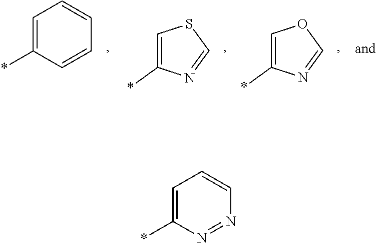 [SMILES: *c1ccccc1 ; *c1cscn1 ; *c1cocn1 ; *c1cccnn1]

wherein the * mark represents a bonding position with L².

In the present disclosure, R¹ is preferably a halogen atom, a C1-6 alkyl group, a C2-6 alkenyl group, a C1-6 alkoxy group, a benzene ring or a 5- to 6-membered heterocyclic ring, and more preferably a halogen atom, a C1-6 alkyl group, a C1-6 alkoxy group, or a benzene ring, and still more preferably a C1-6 alkyl group, or a benzene ring.

In the present disclosure, R^{L1} is preferably a halogen atom, and more preferably a fluorine atom.

In the present disclosure, R^{L2} is preferably a halogen atom, a C1-6 alkyl group, and more preferably a methyl group.

In the present disclosure, R¹⁻¹ is preferably a halogen atom, a C1-6 alkyl group, a C1-6 alkoxy group, a C1-6 haloalkyl group, or a C1-6 haloalkoxy group, and more preferably a halogen atom, a methyl group, or a methoxy group.

In the present disclosure, n is preferably 0, 1, or 2.

In the present disclosure, the compound represented by formula (I) is preferably a compound represented by formula (I-1):

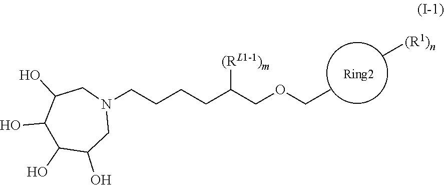

wherein R^{L-1-1} represents (1) a halogen atom, or (2) a C1-6 alkyl group,
m represents an integer of 1 to 2,

Ring 2 represents a ring structure selected from the group consisting of the following ring structures;

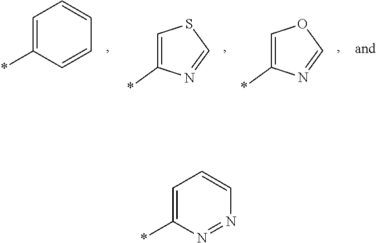 [SMILES: *c1ccccc1 ; *c1cscn1 ; *c1cocn1 ; *c1cccnn1]

wherein the * mark represents a bonding position with methylene, and
other symbols represent the same meaning as the above symbols, and more preferably a compound represented by formula (I-2):

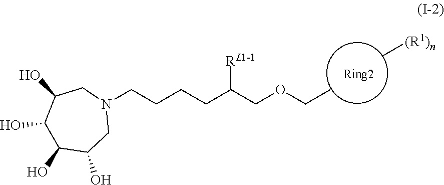

wherein R¹⁻² represents (1) a halogen atom, (2) a C1-6 alkyl group, (3) a C1-6 alkoxy group, or (4) a phenyl group, the groups (2) to (4) in R¹⁻² may be substituted with one to five R¹⁻¹s, and other symbols represent the same meaning as the above symbols.

In the present disclosure, the compound represented by formula (I) is preferably a combination of the preferred definitions of L¹, L², X, Ring 1, R¹, R^{L1}, R^{L2}, R¹⁻¹, and n.

In the present disclosure, the compound represented by formula (I-1) is preferably a combination of the preferred definitions of Ring 2, R¹, R^{L1-1}, R¹⁻¹, m, and n.

In the present disclosure, the compound represented by formula (I-2) is preferably a combination of the preferred definitions of Ring 2, R¹⁻², R^{L1-1}, R¹⁻¹, and n.

In the present disclosure, another embodiment of the compound represented by formula (I) is most preferably an example compound described in Examples described later or a pharmaceutically acceptable salt thereof.

All isomers are included in the present disclosure unless otherwise indicated. For example, the alkyl group, the alkoxy group, the alkylene group, and the like include linear groups and branched groups thereof. In addition, isomers (E-, Z-, cis-, and trans-forms) in a double bond, a ring, and a fused ring, isomers (R-, S-forms, α-, β-forms, enantiomer, and diastereomer) due to the presence of an asymmetric carbon, optically active substances (D-, L-, d-, and l-forms) having optical rotation, polar substances obtained through chromatographic separation (high-polarity substances and low-polarity substances), equilibrium compounds, rotational isomers, mixtures of these at an appropriate ratio, and racemic mixtures are all included in the present disclosure. In addition, all isomers by tautomerism are also included in the present disclosure.

In the present disclosure, unless otherwise specified, the symbol: [symbol]
represents that a substituent is bonded to the back side of the paper surface (that is, α-configuration), the symbol: [symbol]
represents that a substituent is bonded to the front side of the paper surface (that is, 3-configuration), and the symbol binding to chiral carbon atom: [symbol]
represents that a substituent is bonded to the α-configuration, β-configuration or a mixture of α- and β-configurations at an appropriate ratio, as would be apparent to those skilled in the art.

### [Salt]

The compound represented by formula (I) is converted into a salt by a known method.

The salt is a pharmaceutically acceptable salt.

The salt is preferably water-soluble.

Examples of the pharmaceutically acceptable salt include, acid addition salts, alkali metal salts, alkaline earth metal salts, ammonium salts, and amine salts.

Examples of the acid addition salt include inorganic acid salts such as hydrochloride, hydrobromide, hydroiodide, sulfate, phosphate, and nitrate, or organic acid salts such as acetate, lactate, tartrate, benzoate, citrate, methanesulfonate, ethanesulfonate, trifluoroacetate, benzenesulfonate, toluenesulfonate, isethionate, glucuronate, and gluconate.

Examples of the alkali metal salt include a potassium salt and a sodium salt.

Examples of the alkaline earth metal salt include a calcium salt and a magnesium salt.

Examples of the ammonium salt include a tetramethylammonium salt.

Examples of the amine salt include a triethylamine salt, a methylamine salt, a dimethylamine salt, a cyclopentylamine salt, a benzylamine salt, a phenethylamine salt, a piperidine salt, a monoethanolamine salt, a diethanolamine salt, a tris(hydroxymethyl)aminomethane salt, a lysine salt, an arginine salt, and an N-methyl-D-glucamine salt.

In addition, the compound of the present disclosure can be converted into an N-oxide by any method. The N-oxide represents a compound obtained by oxidizing a nitrogen atom of the compound represented by formula (I).

The compound represented by formula (I) and a pharmaceutically acceptable salt thereof may exist in a non-solvated form or in a solvated form with a pharmaceutically acceptable solvent such as water or ethanol. The solvate is preferably a hydrate. The compound represented by formula (I) and a pharmaceutically acceptable salt thereof can be converted into a solvate.

The compound represented by formula (I) can form a cocrystal with an appropriate cocrystal former. The cocrystal is preferably a pharmaceutically acceptable cocrystal that is formed with a pharmaceutically acceptable cocrystal former. The cocrystal is typically defined as a crystal in which two or more different molecules are formed by intermolecular interaction that is different from ionic bonds. The cocrystal may be a complex of a neutral molecule and a salt. The cocrystal can be prepared by a known method, for example, by melting crystallization, recrystallization from a solvent, or physically pulverizing the components together. Suitable cocrystal formers include those described in WO 2006/007448.

In the present disclosure, all references to the compound of the present disclosure include a compound represented by formula (I), a pharmaceutically acceptable salt thereof, an N-oxide thereof, a solvate thereof (for example, a hydrate), or a cocrystal thereof, or an N-oxide of a pharmaceutically acceptable salt of the compound represented by formula (I), a solvate thereof (for example, a hydrate), or a cocrystal thereof.

### [Prodrug]

The prodrug of the compound represented by formula (I) refers to a compound that is converted into the compound represented by formula (I) through a reaction with an enzyme, gastric acid, or the like in vivo. Examples of the prodrug of the compound represented by formula (I) include: compounds in which an amino group is acylated, alkylated, or phosphorylated (for example, compounds in which the amino group of the compound represented by formula (I) is eicosanoylated, alanylated, pentylaminocarbonylated, (5-methyl-2-oxo-1,3-dioxolen-4-yl)methoxycarbonylated, tetrahydrofuranylated, pyrrolidyl methylated, pivaloyloxymethylated, acetoxymethylated, tert-butylated, or the like) in the case where the compound represented by formula (I) has an amino group; compounds in which a hydroxyl group is acylated, alkylated, phosphorylated, or borated (for example, compounds in which the hydroxyl group of the compound represented by formula (I) is acetylated, palmitoylated, propanoylated, pivaloylated, succinylated, fumarylated, alanylated, dimethylaminomethylcarbonylated, or the like) in the case where the compound represented by formula (I) has a hydroxyl group; and compounds in which a carboxy group is esterified or amidated (for example, compounds in which the carboxy group of the compound represented by formula (I) is ethyl esterified, phenyl esterified, carboxymethyl esterified, dimethylaminomethyl esterified, pivaloyloxymethyl esterified, 1-{(ethoxycarbonyl)oxy}ethyl esterified, phthalidyl esterified, (5-methyl-2-oxo-1,3-dioxolen-4-yl)methyl esterified, 1-{[(cyclohexyloxy)carbonyl]oxy}ethyl esterified, methylamidated, or the like) in the case where the compound represented by formula (I) has a carboxy group. These compounds can be produced by a method known per se. The prodrug of the compound represented by formula (I) may be either a hydrate or a non-hydrate. In addition, the prodrug of the compound represented by formula (I) may be a compound that is converted into the compound represented by formula (I) under a physiological condition as described in “Development of Pharmaceuticals”, Vol. 7, “Molecular Design”, pp. 163 to 198, published by Hirokawa Shoten, 1990.

Furthermore, each atom constituting the compound represented by formula (I) may be substituted with an isotope thereof (for example, ²H, ³H, ¹¹C, ¹³C, ¹⁴C, ¹⁵N, ¹⁶N, ¹⁷O, ¹⁸O, ¹⁸F, ³⁵S, ³⁶Cl, ⁷⁷Br, and ¹²⁵I) or the like.

### [Method for Producing Compound of Present Disclosure]

The compound of the present disclosure can be produced by appropriately improving known methods, for example, methods shown below, methods equivalents thereto, the method described in Comprehensive Organic Transformations: A Guide to Functional Group Preparations, 3rd Edition (Richard C. Larock, John Wiley & Sons Inc, 2018), the methods described in Examples or the like, and using a combination thereof. A salt may be used as the starting material thereof. The order of carrying out each reaction can be appropriately changed depending on the protecting group introduced and reaction conditions.

In addition, a compound having an amino group, a carboxyl group, or a hydroxyl group can be produced, if necessary, by performing a known deprotection reaction after an appropriate reaction step, by using a compound protected with a protecting group commonly used for these groups. The protecting group is, for example, the protecting groups described in T. W. Greene, Protective Groups in Organic Synthesis, Wiley, New York, 5th Edition, 2014.

Examples of the protecting group of a carboxyl group include methyl, ethyl, tert-butyl, trichloroethyl, benzyl (Bn), phenacyl, p-methoxybenzyl, trityl, and 2-chlorotrityl.

Examples of the protecting group of an amino group or a tetrazolyl group include a benzyloxycarbonyl group, a tert-butoxycarbonyl group, an allyloxycarbonyl (Alloc) group, a 1-methyl-1(4-biphenyl)ethoxycarbonyl (Bpoc) group, a trifluoroacetyl group, a 9-fluorenylmethoxycarbonyl group, a benzyl (Bn) group, a p-methoxybenzyl group, a benzyloxymethyl (BOM) group, and a 2-(trimethylsilyl)ethoxymethyl (SEM) group.

Examples of the protecting group of a hydroxyl group or hydroxamic acid include methyl, trityl, methoxymethyl (MOM), 1-ethoxyethyl (EE), methoxyethoxymethyl (MEM), 2-tetrahydropyranyl (THP), trimethylsilyl (TMS), triethylsilyl (TES), tert-butyldimethylsilyl (TBDMS), tert-butyldiphenylsilyl (TBDPS), acetyl (Ac), pivaloyl, benzoyl, benzyl (Bn), p-methoxybenzyl, allyloxycarbonyl (Alloc), and 2,2,2-trichloroethoxycarbonyl (Troc).

The deprotection reaction is known, and can be performed by, for example, the following methods: (1) a deprotection reaction by alkaline hydrolysis;
(2) a deprotection reaction under an acidic condition;
(3) a deprotection reaction by hydrogenolysis;
(4) a deprotection reaction of a silyl group;
(5) a deprotection reaction by using a metal; and
(6) a deprotection reaction by using a metal complex.

These methods are specifically described as follows.

(1) The deprotection reaction by alkaline hydrolysis is performed, for example, at 0 to 40° C., in an organic solvent (for example, methanol, tetrahydrofuran (hereinafter, THF), and dioxane), by using a hydroxide of an alkali metal (for example, sodium hydroxide, potassium hydroxide, and lithium hydroxide), a hydroxide of an alkaline earth metal (for example, barium hydroxide and calcium hydroxide), a carbonate (for example, sodium carbonate and potassium carbonate), or an aqueous solution or mixture thereof.

(2) The deprotection reaction under an acidic condition is performed, for example, at 0 to 100° C., in an organic solvent (for example, dichloromethane, chloroform, dioxane, ethyl acetate, methanol, isopropyl alcohol, THF, and anisole), in an organic acid (for example, acetic acid, trifluoroacetic acid, methanesulfonic acid, and p-tosylic acid) or an inorganic acid (for example, hydrochloric acid and sulfuric acid), or a mixture thereof (for example, hydrogen bromide/acetic acid), in the presence or absence of 2,2,2-trifluoroethanol.

(3) The deprotection reaction by hydrogenolysis is performed, for example, at 0 to 200° C., in a solvent (for example, an ether-based solvent such as THF, dioxane, dimethoxyethane, and diethyl ether; an alcohol-based solvent such as methanol and ethanol; a benzene-based solvent such as benzene and toluene; a ketone-based solvent such as acetone and methyl ethyl ketone; a nitrile-based solvent such as acetonitrile; an amide-based solvent such as N,N-dimethylformamide (hereinafter, DMF); water, ethyl acetate, acetic acid, or a mixed solvent of two or more thereof), in the presence of a catalyst (for example, palladium-carbon, palladium black, palladium hydroxide-carbon, platinum oxide, and Raney nickel), under a hydrogen atmosphere under normal pressure or pressure, or in the presence of ammonium formate.

(4) The deprotection reaction of a silyl group is performed, for example, at 0 to 40° C., in an organic solvent (for example, THF and acetonitrile) miscible with water, by using tetrabutylammonium fluoride. In addition, the deprotection reaction is performed, for example, at −10 to 100° C., in an organic acid (for example, acetic acid, trifluoroacetic acid, methanesulfonic acid, and p-tosylic acid) or an inorganic acid (for example, hydrochloric acid and sulfuric acid), or a mixture thereof (for example, hydrogen bromide/acetic acid).

(5) The deprotection reaction using a metal is performed, for example, at 0 to 40° C., in an acidic solvent (for example, a mixed solution of acetic acid, a buffer solution having a pH of 4.2 to 7.2, or a solution thereof with an organic solvent such as THF), in the presence of powdery zinc while applying ultrasonic waves if necessary.

(6) The deprotection reaction using a metal complex is performed, for example, at 0 to 40° C., in an organic solvent (for example, dichloromethane, DMF, THF, ethyl acetate, acetonitrile, dioxane, and ethanol), water, or a mixed solvent thereof, in the presence of a trapping reagent (for example, tributyltin hydride, triethylsilane, dimedone, morpholine, diethylamine, and pyrrolidine), an organic acid (for example, acetic acid, formic acid, and 2-ethylhexanoic acid), and/or an organic acid salt (for example, sodium 2-ethylhexanoate and potassium 2-ethylhexanoate), in the presence or absence of a phosphine-based reagent (for example, triphenylphosphine), by using a metal complex (for example, tetrakistriphenylphosphine palladium(0), bis(triphenylphosphine)palladium(II) dichloride, palladium(II) acetate, and tris(triphenylphosphine)rhodium(I) chloride).

The compound represented by formula (I) can be produced by Reaction Scheme 1.

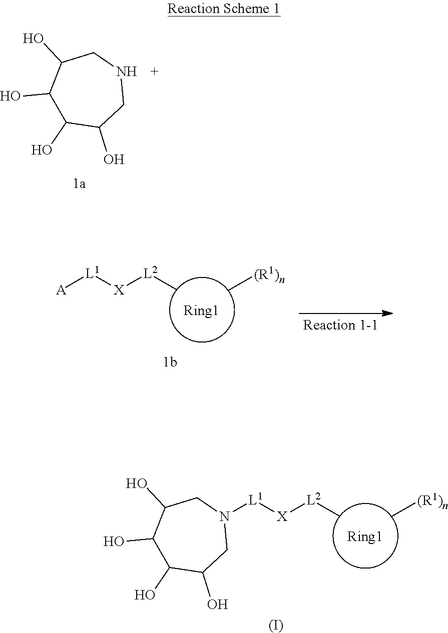

Wherein A represents a leaving group (for example, halogen atom, aryl sulfonate or alkyl sulfonate), and other symbols represent the same meaning as described above.

In Reaction Scheme 1, Reaction 1-1 is an alkylation reaction of amino group. The alkylation reaction of amino group is known, and is performed, for example, through reaction at 0 to 200° C., in an organic solvent (DMF, dimethyl sulfoxide, chloroform, dichloromethane, diethyl ether, THF, methyl t-butyl ether, and the like), in the presence of a base (triethylamine, diisopropylethylamine, sodium carbonate, sodium bicarbonate, potassium carbonate, cesium carbonate, tripotassium phosphate, cesium fluoride, barium hydroxide, tetrabutylammonium fluoride, and the like), in the presence of an inorganic salts (potassium iodide) or an aqueous solution or mixture thereof.

The compound represented by formula (1b) can be produced by Reaction Scheme 2.

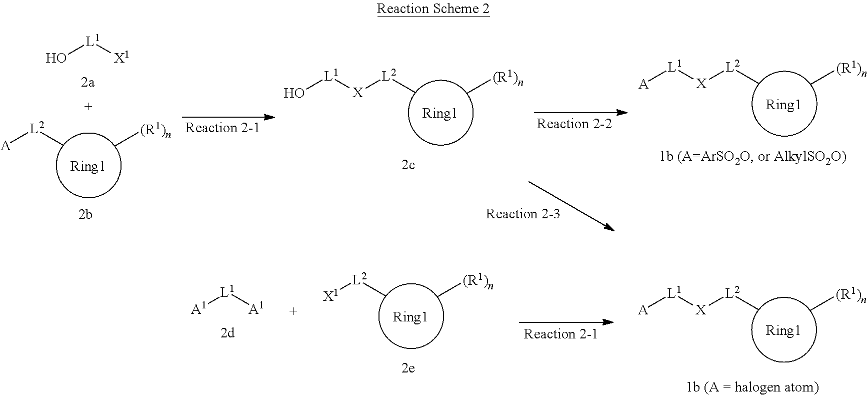

Wherein X¹ represents OH or SH, A¹ represents halogen atom, and other symbols represent the same meaning as described above.

In Reaction Scheme 2, Reaction 2-1 is an alkylation reaction of hydroxyl group or thiol group. The alkylation reaction of hydroxyl group is known, and is performed, for example, through reaction at 0 to 200° C., in an organic solvent (DMF, dimethyl sulfoxide, chloroform, dichloromethane, diethyl ether, THF, methyl t-butyl ether, and the like), in the presence of a base (triethylamine, diisopropylethylamine, sodium carbonate, sodium bicarbonate, potassium carbonate, cesium carbonate, tripotassium phosphate, cesium fluoride, barium hydroxide, tetrabutylammonium fluoride, potassium t-butoxide, sodium hydride and the like), in the presence of an inorganic salts (potassium iodide) in the presence of a phase transfer catalyst (tetrabutylammonium bromide, tetrabutylammonium iodide and the like) or an aqueous solution or mixture thereof.

In Reaction Scheme 2, Reaction 2-2 is a sulfonylation reaction of hydroxyl group. The sulfonylation reaction of hydroxyl group is known, and is performed, for example, through reaction at 0 to 100° C., in an organic solvent (acetonitrile, chloroform, dichloromethane, pyridine, and the like), in the presence of a sulfonylation agent (p-toluenesulfonyl chloride, methanesulfonyl chloride, methanesulfonic anhydride and lithium bromide and the like), in the presence or absence of a base (triethylamine, diisopropylethylamine, sodium carbonate, sodium bicarbonate, potassium carbonate and the like) or an aqueous solution or mixture thereof.

In Reaction Scheme 2, Reaction 2-3 is a halogenation reaction of hydroxyl group. The halogenation reaction of hydroxyl group is known, and is performed, for example, through reaction at 0 to 100° C., in an organic solvent (THF, toluene, chloroform, dichloromethane, pyridine and the like), in the presence of a halogenation agent (carbon tetrabromide, triphenylphosphine, phosphorous tribromide, methanesulfonic anhydride lithium bromide and the like), in the presence of a base (triethylamine, diisopropylethylamine, sodium carbonate, sodium bicarbonate, potassium carbonate and the like) or an aqueous solution or mixture thereof.

The compound represented by formula (3b) can be produced by Reaction Scheme 3.

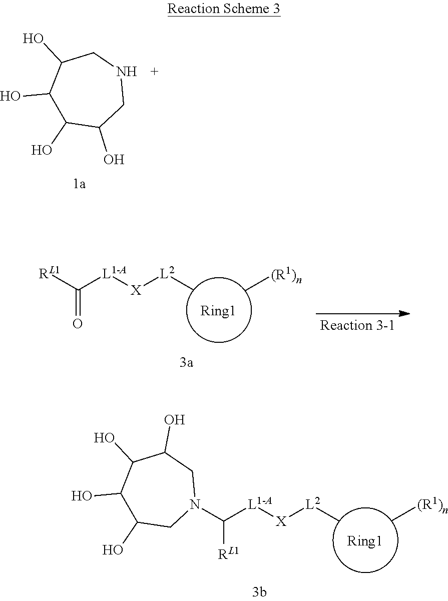

Wherein L^{1-A} represents (1) a C3-6 alkylene, (2) a C3-6 alkenylene, or (3) a C3-6 alkynylene, and other symbols represent the same meaning as described above.

In Reaction Scheme 3, Reaction 3-1 is a reductive amination. The reductive amination is known, and is performed, for example, through reaction at a temperature of 0 to 40° C. in an organic solvent (e.g., dichloroethane, dichloromethane, dimethylformamide, acetic acid, and a mixture thereof) in the presence of a reducing agent (e.g., sodium triacetoxyborohydride, sodium cyanoborohydride, sodium borohydride).

In one aspect, a compound corresponding to the compound represented by formula (4 g) can be produced by Reaction Scheme 4.

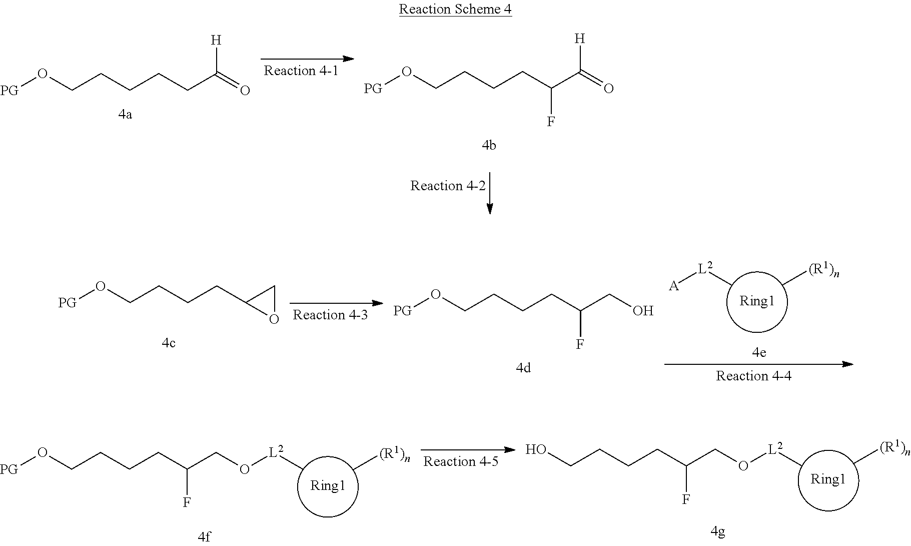

Wherein PG represents a protecting group, and other symbols represent the same meaning as described above.

In Reaction Scheme 4, Reaction 4-1 is a fluorination reaction. The fluorination reaction is known, and is performed, for example, through reaction at a temperature of −20 to 40° C., in an organic solvent (e.g., THF, acetonitrile, EtOAc, chloroform, isopropyl alcohol and a mixture thereof), in the presence of hydrogen fluoride salts (e.g. triethylamine tris(hydrogen fluoride), pyridine hydrogen fluoride, potassium hydrogen bifluoride and the like), in the presence of organic catalyst (DL-proline, L-proline, D-proline, (5R)-(+)-2,2,3-Trimethyl-5-benzyl-4-imidazolidinone dichloroacetic acid, (5S)-(+)-2,2,3-Trimethyl-5-benzyl-4-imidazolidinone dichloroacetic acid and the like).

In Reaction Scheme 4, Reaction 4-2 is a reduction reaction of an aldehyde group, and is performed at 0 to 40° C., in an organic solvent (dichloroethane, dichloromethane, DMF, THF, a mixture thereof, and the like), in the presence of a reducing agent (such as sodium borohydride).

In Reaction Scheme 4, Reaction 4-3 is a fluorination reaction. The fluorination reaction is known, and is performed, for example, through reaction at a temperature of −20 to 70° C., in an organic solvent (e.g., dichloromethane, chloroform, 1,2-dichloro-ethane, toluene, diethyl ether and a mixture thereof), in the presence of hydrogen fluoride salts (e.g. triethylamine tris(hydrogen fluoride), pyridine hydrogen fluoride, potassium hydrogen bifluoride and the like).

In Reaction Scheme 4, Reaction 4-4 is an alkylation reaction of hydroxyl group which is known and is performed as described in Reaction Scheme 2, Reaction 2-1.

In Reaction Scheme 4, Reaction 4-5 is a deprotection reaction of protecting group which is known and is performed as described above.

In one aspect, a compound corresponding to the compound represented by formula (5d) can be produced by Reaction Scheme 5.

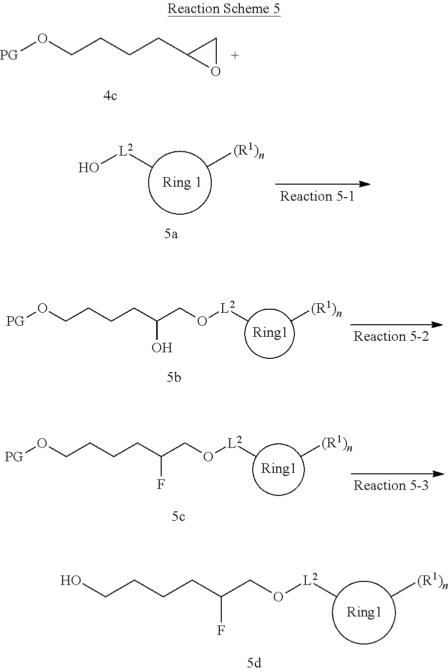

Wherein PG represents a protecting group, and other symbols represent the same meaning as described above.

In Reaction Scheme 5, Reaction 5-1 is an ether formation reaction. The ether formation reaction is known, and is performed, for example, through reaction at a temperature of 20 to 100° C., in an organic solvent (e.g., t-BuOH, H₂O and a mixture thereof), in the presence of inorganic base (e.g. sodium hydroxide and the like) in the presence of a phase transfer catalyst (tetrabutylammonium iodide and the like).

In Reaction Scheme 5, Reaction 5-2 is a fluorination reaction. The fluorination reaction is known, and is performed, for example, through reaction at a temperature of −20 to 40° C., in an organic solvent (e.g., dichloromethane, THF, toluene and a mixture thereof), in the presence of fluorination agent (e.g. diethylamino-sulfur trifluoride, perfluorobutanesulfonyl fluoride, 2-pyridinesulfonyl fluoride and the like), in the presence or absence of a base (triethylamine, diisopropylethylamine, sodium carbonate, sodium bicarbonate, potassium carbonate and the like), in the presence or absence of hydrogen fluoride salts (e.g. triethylamine tris(hydrogen fluoride)).

In Reaction Scheme 5, Reaction 5-3 is a deprotection reaction of protecting group which is known and is performed as described above.

In one aspect, a compound corresponding to the compound represented by formula (6k) can be produced by Reaction Scheme 6.

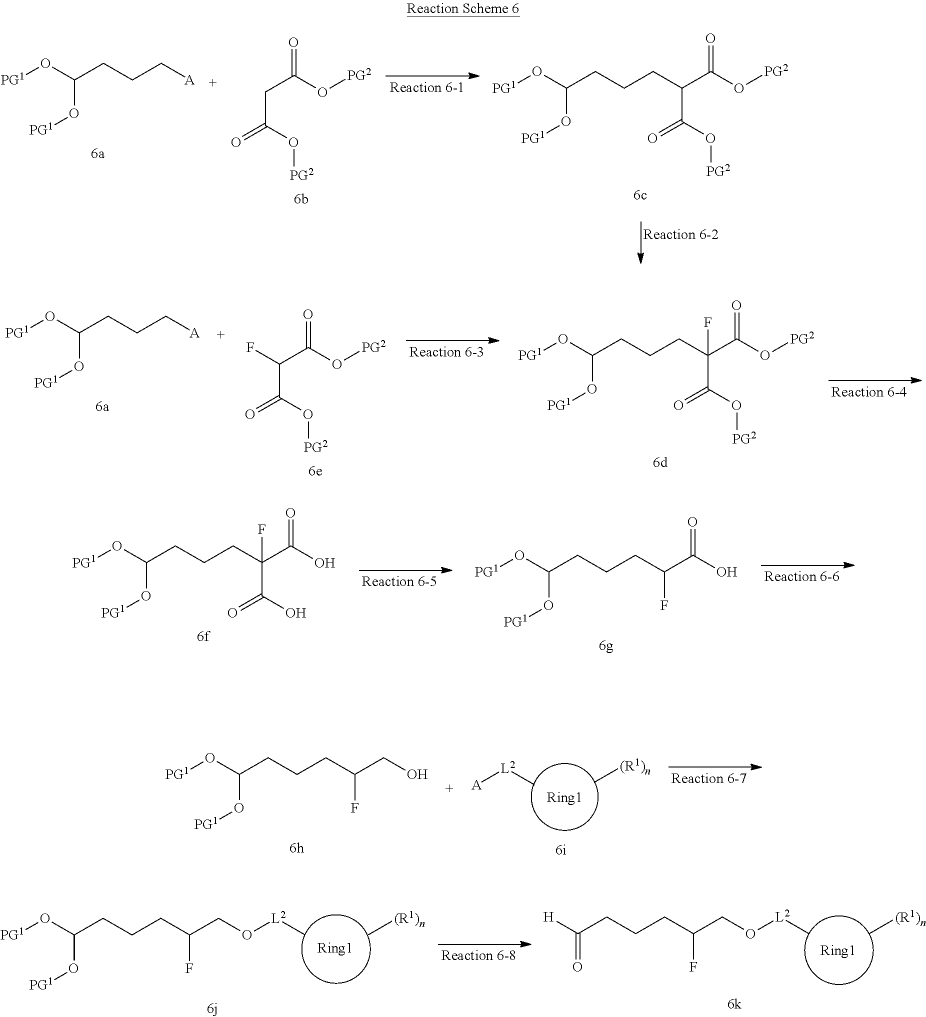

Wherein PG¹ represents a protecting group of aldehyde, PG² represents a protecting group of carboxylic acid, and other symbols represent the same meaning as described above.

In Reaction Scheme 6, Reaction 6-1 is an alkylation reaction of malonic acid diester. The alkylation reaction of malonic acid diester is known, and is performed, for example, through reaction at a temperature of 0 to 100° C., in an organic solvent (DMF, dimethyl sulfoxide, chloroform, dichloromethane, diethyl ether, THF, methyl t-butyl ether, and the like), in the presence of a base (e.g., sodium hydride, potassium t-butoxide, potassium carbonate and the like).

In Reaction Scheme 6, Reaction 6-2 is a fluorination reaction. The fluorination reaction is known, and is performed, for example, through reaction at a temperature of 0 to 100° C., in an organic solvent (diethyl ether, THF, methyl t-butyl ether, acetonitrile, DMF and the like), in the presence of fluorination agent (e.g. 1-(chloromethyl)-4-fluoro-1,4-diazoniabicyclo-[2.2.2]octane bis(tetrafluoroborate), N-fluoro-bis[(trifluoromethyl)sulfonyl]imide, N-fluorodibenzenesulfonimide and the like), in the presence or absence of a base (sodium hydride, lithium diisopropyl amide, cesium carbonate and the like).

In Reaction Scheme 6, Reaction 6-3 is an alkylation reaction of 2-fluoro malonic acid diester which is known and is performed as described in Reaction 6-1.

In Reaction Scheme 6, Reaction 6-4 is a deprotection reaction of protecting group which is known and is performed as described above.

In Reaction Scheme 6, Reaction 6-5 is a decarboxylation reaction. The thermal decarboxylation reaction is known, and is performed, for example, through reaction at a temperature of 20 to 200° C., in an organic solvent (DMF, dimethyl sulfoxide, pyridine and the like), in the presence or absence of a base (for example, triethylamine, diisopropylethylamine, lithium hydroxide, sodium hydroxide, sodium carbonate, sodium bicarbonate, potassium hydroxide, potassium carbonate, cesium carbonate, tripotassium phosphate, cesium fluoride, barium hydroxide, tetrabutylammonium fluoride), in the presence or absence of an organic acid (for example, acetic acid, trifluoroacetic acid, methanesulfonic acid, and p-tosylic acid) or an inorganic acid (for example, hydrochloric acid and sulfuric acid), or a mixture thereof.

In Reaction Scheme 6, Reaction 6-6 is a reduction of carboxylic acid. The reduction of carboxylic acid is known, and is performed, for example, through reaction at a temperature of 20 to 200° C., in an organic solvent (diethyl ether, THF, methyl t-butyl ether, and the like), in the presence of reducing agent (e.g. BH₃—SMe₂, BH₃-THF, LiAlH₄, and the like).

In Reaction Scheme 6, Reaction 6-7 is an alkylation reaction of hydroxyl group which is known and is performed as described in Reaction Scheme 2, Reaction 2-1.

In Reaction Scheme 6, Reaction 6-8 is a deprotection reaction of protecting group which is known and is performed as described above (for example, a deprotection reaction under an acidic condition).

In each reaction in the present specification, the compounds represented by formula 1a, 2a, 2b, 2d, 2e, 3a, 4a, 4c, 4e, 5a, 6a, 6b, 6e, and formula 6i, used as starting materials are known, or can be easily produced by combining known methods, for example, the methods described in Comprehensive Organic Transformations: A Guide to Functional Group Preparations, 3rd Edition (Richard C. Larock, John Wiley & Sons Inc, 2018) and the like, or methods obtained by partially modifying known methods or the like.

Among the compounds of the present disclosure, a compound having optical activity can be produced by using a starting material or reagent having optical activity, or by optically separating a racemic intermediate and then converting to the present disclosure compound therefrom, or optically separating a racemic form of the compound of the present disclosure.

This optical separation is known, and examples thereof include a method in which a salt, a complex, or the like is formed with another optically active compound, and then a target compound is isolated after recrystallization or directly separated using a chiral column or the like.

In the reactions in the present specification, a reaction involving heating can be performed using a water bath, an oil bath, a sand bath, or a microwave, as would be apparent to those skilled in the art.

In the reactions in the present specification, a solid-phase supported reagent that is supported on a polymer (for example, polystyrene, polyacrylamide, polypropylene, polyethylene glycol, and the like) may be appropriately used.

In the reactions in the present specification, the reaction product can be purified by ordinary purification means, for example, distillation under normal pressure or reduced pressure, high-performance liquid chromatography using silica gel or magnesium silicate, thin-layer chromatography, methods using an ion-exchange resin or a scavenger resin, column chromatography, washing, and recrystallization. The purification may be performed for each reaction or may be performed after completion of some reactions.

### [Toxicity]

The toxicity of the compound of the present disclosure is low, and therefore can be used as a medicament safely.

### [Application to Medicament]

The compound of the present disclosure has GBA1 and GCS dual modulatory activity, and thus the compound of the present disclosure is useful as an agent for preventing and/or treating diseases associated with GBA1 and/or GCS, for example, Parkinson's disease, Lewy body disease, Gaucher disease, multiple system atrophy, Fabry disease, Niemann-Pick disease type C, lysosomal storage diseases, dementia, epilepsy, bipolar disorder, schizophrenia, anxiety disorder, major depression disorder, Sandhoffs disease, Tay-Sachs disease, multiple sclerosis, Huntington's disease, Alzheimer's disease, amyotrophic lateral sclerosis, frontotemporal lobar degeneration, cognitive impairment, learning disabilities, gait disorders, sleeping disorder, neurological disorders, CNS disorders, polycystic kidney disease, type 2 diabetes, cancer, open angle glaucoma, endometriosis, multiple myeloma, virus infection, inflammation, systemic lupus erythematosus, hepatitis, Guillain-Barre Syndrome, diabetic nephropathy, skin disorder.

The disease associated with GBA1 and/or GCS is preferably Parkinson's disease, Lewy body disease, or Gaucher disease.

In the use of the compound of the present disclosure for the purpose of preventing and/or treating the above-mentioned diseases, the substance that acts as an active ingredient, is usually formulated together with a pharmaceutically acceptable carrier such as various additives or solvents, and then administered systemically or topically, orally or parenterally. Here, the pharmaceutically acceptable carrier means a substance other than the active ingredient, which is generally used for pharmaceutical preparation. The pharmaceutically acceptable carrier preferably does not exhibit a pharmacological action at the dosage of the preparation, is harmless, and does not interfere with the therapeutic effect of the active ingredient. The pharmaceutically acceptable carrier can also be used for the purpose of, for example, enhancing the usefulness of the active ingredient and the preparation, facilitating the formulation, stabilizing the quality, or improving the usability. Specifically, substances described in “Japanese Pharmaceutical Excipients Directory” published in 2021 by Yakuji Nippo Limited (edited by International Pharmaceutical Excipients Council Japan) and the like may be appropriately selected according to the purpose.

The compound of the present disclosure can be administered to a mammal (preferably human, more preferably a human patient) in a pharmaceutically effective amount.

Since the dosage of the compound of the present disclosure depends on age, body weight, symptom, desired therapeutic effect, route of administration, period of treatment, and the like, the dosage inevitably varies. The compound of the present disclosure is generally administered orally in the range of 0.1 ng to 1,000 mg per patient per administration, or parenterally administered or continuously administered intravenously in the range of 0.01 ng to 100 mg per patient per administration.

Needless to say, the dosage varies depending on various conditions as described above. Therefore, a dosage smaller than the above dosage may be sufficient, or a dosage beyond the above range may be required.

Examples of the dosage form thereof include preparations for oral administration (for example, tablets, capsules, granules, powders, oral solutions, syrups, and oral jellies), preparations for oral cavity (for example, oral tablets, oral sprays, oral semisolids, and oral rinses), preparations for injection (for example, injectables), preparations for dialysis (for example, dialysis agents), preparations for inhalation (for example, inhalants), preparations for ophthalmic application (for example, eye drops and ophthalmic ointments), preparations for otologic application (for example, ear drops), preparations for nasal application (for example, nose drops), preparations for rectum (for example, suppositories, semisolids for rectum, and enemas for rectal application), preparations for vagina (for example, vaginal tablets and vaginal suppositories), and preparations for skin (for example, external solid agents, external liquid agents, sprays, ointments, creams, gels, and patches).

### [Preparation for Oral Administration]

Examples of the preparation for oral administration include tablets, capsules, granules, powders, oral solutions, syrups, and oral jellies. The preparation for oral administration includes rapidly disintegrating preparations in which the releasability of the active ingredient from the preparation is not particularly adjusted; and controlled-release preparations such as enteric-coated preparations and sustained-release preparations in which the releasability is adjusted according to the purpose by unique formulation design and formulation method. The enteric-coated preparation refers to a preparation designed to release the active ingredient mainly in the small intestine without releasing the active ingredient in the stomach, for the purpose of preventing the degradation of the active ingredient in the stomach or reducing the irritation of the active ingredient to the stomach. The enteric-coated preparation can be usually produced by coating with an acid-insoluble enteric-coated base. The sustained-release preparation refers to a preparation in which the release rate, release time, and release site of the active ingredient from the preparation are adjusted for the purpose of reducing the number of administrations or attenuating side effects. The sustained-release preparation can be usually produced by using an appropriate sustained-release agent. Among preparations for oral administration, capsules, granules, tablets, and the like can be coated with an appropriate coating agent such as a saccharide, a sugar alcohol, or a polymer compound, for the purpose of, for example, facilitating ingestion or preventing degradation of the active ingredient.

### (1) Tablet

The tablet is a solid preparation that is administered orally and has a certain shape. The tablet includes those generally referred to as tablets, such as uncoated tablets, film-coated tablets, sugar-coated tablets, multilayer tablets, and dry-coated tablets, as well as orally rapidly disintegrating tablets, chewable tablets, effervescent tablets, dispersible tablets, and soluble tablets. In the production of the uncoated tablet, the following method (a), (b), or (c) is usually used:
(a) Additives such as an excipient, a binder, and a disintegrant are added to the active ingredient and mixed to prepare a homogeneous mixture, the mixture is granulated by an appropriate method using water or a solution containing a binder, then a lubricant or the like is added to the granulated product and mixed, and the resulting mixture was compression-molded;
(b) Additives such as an excipient, a binder, and a disintegrant are added to the active ingredient and mixed to prepare a homogeneous mixture, and the mixture is directly compression-molded; or the active ingredient, a lubricant, and the like are added to granules prepared in advance with additives to prepare a homogeneous mixture, and then the mixture is compression-molded; and
(c) Additives such as an excipient and a binder are added to the active ingredient and mixed to prepare a homogeneous mixture, a kneaded product obtained by wetting the mixture with a solvent is poured into a predetermined mold and molded, and then the molded product is dried by an appropriate method.

The film-coated tablet can be usually produced by thinly coating an uncoated tablet with an appropriate coating agent such as a polymer compound. The sugar-coated tablet can be usually produced by coating an uncoated tablet with a coating agent containing a saccharide or a sugar alcohol. The multilayer tablet can be produced by forming layers of powder particles having different compositions and compression-molding the layered powder particles by an appropriate method. The dry-coated tablet can be produced by covering an inner core tablet with an outer layer having a composition different from that of the inner core tablet. The tablet can also be an enteric-coated tablet or a sustained-release tablet by a known appropriate method. The orally rapidly disintegrating tablet, the chewable tablet, the effervescent tablet, the dispersible tablet, and the soluble tablet are those in which unique functions are imparted to the tablets by appropriate selection of additives, and can be produced according to the above-described methods for producing the tablet. Incidentally, the orally rapidly disintegrating tablet refers to a tablet that can be taken by being rapidly dissolved or disintegrated in the oral cavity; the chewable tablet refers to a tablet that is chewed and taken; the effervescent tablet refers to a tablet that dissolves or disperses while rapidly foaming in water; the dispersible tablet refers to a tablet that is taken by being dispersed in water; and the soluble tablet refers to a tablet that is taken by being dissolved in water. The effervescent tablet can be produced by using an appropriate acidic substance, a carbonate, a bicarbonate, or the like as an additive.

### (2) Capsule

The capsule is a preparation in which ingredients are filled in a capsule or encapsulated with a capsule base, and includes hard capsules, soft capsules, and the like. The hard capsule can be produced by adding an additive such as an excipient to the active ingredient and mixing them to prepare a homogeneous mixture, or forming the mixture into particles or a molded product by an appropriate method, and filling, in a capsule, the mixture, the particles, or the molded product as it is or after each content is slightly molded. The soft capsule can be produced by encapsulating a mixture of the active ingredient and an additive in a certain shape with an appropriate capsule base such as gelatin having increased plasticity by adding glycerin, D-sorbitol, or the like. The capsule may be an enteric-coated capsule or a sustained-release capsule prepared by a known appropriate method, and a colorant, a preservative, or the like may be added to the capsule base.

### (3) Granule

The granule is a preparation granulated in a granular form, and includes effervescent granules and the like in addition to those generally referred to as granules. In the production of the granule, the following method (a), (b), or (c) is usually used:
(a) An excipient, a binder, a disintegrant, or other additives are added to a powdery active ingredient and mixed to prepare a homogeneous mixture, and then the mixture is granulated by an appropriate method;
(b) An additive such as an excipient is added to the active ingredient prepared in a granular form in advance, and mixed to prepare a homogeneous mixture; and
(c) An additive such as an excipient is added to the active ingredient prepared in a granular form in advance and mixed, and the mixture is then formed into granules by an appropriate method.

The granule may be coated as necessary, and may be an enteric-coated granule or a sustained-release granule prepared by a known appropriate method. The effervescent granule can be produced by using an appropriate acidic substance, a carbonate, a bicarbonate, or the like as an additive. The effervescent granule refers to a granule that dissolves or disperses while rapidly foaming in water. The granule can also be made into a fine granule by adjusting the size of the particle.

### (4) Powder

The powder is a powdery preparation, and can be usually produced by adding an excipient or other additives to the active ingredient and mixing them to prepare a homogeneous mixture.

### (5) Oral Solution

The oral solution is a liquid or flowable viscous gel preparation, and includes, in addition to those generally referred to as oral solutions, an elixir, a suspension, an emulsion, a lemonade, and the like. The oral solution can be usually produced by adding an additive and purified water to the active ingredient, followed by mixing, uniformly dissolving or emulsifying or suspending, and filtering as necessary. The elixir refers to a clear liquid oral solution containing sweet and aromatic ethanol. The elixir can be usually produced by adding ethanol, purified water, a flavoring agent, and white sugar, other saccharides or a sweetener to a solid active ingredient or a leachate thereof, dissolving components, and preparing a clear liquid by filtration or other methods. The suspension refers to an oral solution in which the active ingredient is finely and homogeneously suspended. The suspension can be usually produced by adding a suspending agent or other additives and purified water or oil to a solid active ingredient, and performing suspension by an appropriate method to homogenize the entire active ingredient. The emulsion refers to an oral solution in which the active ingredient is finely and homogeneously emulsified. The emulsion can be usually produced by adding an emulsifier and purified water to a liquid active ingredient, and performing emulsification by an appropriate method to homogenize the mixture. The lemonade refers to a clear liquid oral solution having a sweet taste and a sour taste.

### (6) Syrup

The syrup is a viscous liquid preparation or solid preparation containing a saccharide or sweetener, and includes preparations for syrup and the like. The syrup can be usually produced by adding the active ingredient to a solution of white sugar, other saccharides or a sweetener, or a single syrup, followed by dissolving, mixing, suspending, or emulsifying, and if necessary, boiling the mixed liquid, followed by hot filtration. The preparation for syrup refers to a granular or powdery preparation that becomes a syrup when water is added, and may also be referred to as a dry syrup. The preparation for syrup can be usually produced using a saccharide or a sweetener as an additive in accordance with the method for producing the granule or powder.

### (7) Oral Jelly

The oral jelly is a formed gel preparation having no fluidity, and can be usually produced by adding an additive and a polymer gel base to the active ingredient and mixing them, gelatinizing the mixture by an appropriate method, and forming the resulting gel into a certain shape.

### [Preparation for Injection]

### (1) Injectable

The injectable is a solution, a suspension or an emulsion, or a solid sterile preparation to be dissolved or suspended when used, which is administered subcutaneously, intramuscularly, or directly administered to a body tissue such as blood vessel or an organ. The injectable includes a lyophilized injectable, a powder injectable, a filled syringe, a cartridge, an infusion, an embedded injectable, a long acting injectable, and the like, in addition to those generally referred to as injectables. In the production of the injectable, the following method (a), (b), or (c) is usually used:
(a) A homogeneous solution is prepared by dissolving, suspending, or emulsifying the active ingredient as it is or a mixture obtained by adding an additive to the active ingredient, in water for injection, another aqueous solvent, a non-aqueous solvent or the like. Then, the prepared solution is filled in a container for injection, and then the container is sealed, followed by sterilization; and
(b) A homogeneous solution is prepared by dissolving, suspending, or emulsifying the active ingredient as it is or a mixture obtained by adding an additive to the active ingredient, in water for injection, another aqueous solvent, a non-aqueous solvent or the like. Then, the prepared solution is aseptically filtrated or aseptically prepared, the resulting solution is filled in a container for injection, and the container is sealed.

The lyophilized injectable can be usually produced by dissolving the active ingredient as it is or a mixture of the active ingredient and an additive such as an excipient, in water for injection, aseptically filtering the solution, filling the solution in a container for injection, and then lyophilizing the solution, or directly filling the solution in a container after lyophilizing the solution in a dedicated container. The powder injectable can be usually produced by crystallizing a powder subjected to aseptic filtration to prepare a powder, and if necessary, adding a sterilized additive to the prepared powder, and filling the powder in a container for injection. A filled syringe can be usually produced by preparing a solution, a suspension, or an emulsion with the active ingredient as it is or with the active ingredient and an additive, and then filling the prepared solution, suspension, or emulsion in a glass syringe. The cartridge refers to an injectable used by inserting a cartridge filled with a drug solution into a dedicated syringe. The cartridge filled with the drug solution can be usually produced by preparing a solution, a suspension, or an emulsion with the active ingredient as it is or with the active ingredient and an additive, and filling the prepared solution, suspension, or emulsion into the cartridge. The infusion refers to 100 mL or more of injectable usually administered intravenously. The embedded injectable refers to a solid or gel-like injection which is applied subcutaneously, intramuscularly or the like by using an implantation tool or by surgery, for the purpose of releasing the active ingredient over a long period of time. The embedded injectable can be usually produced by using a biodegradable polymer compound and forming the compound into a pellet, microsphere, or gel. The long acting injectable refers to an injectable which is applied intramuscularly or the like for the purpose of releasing the active ingredient over a long period of time. The sustained injectable can be usually produced by dissolving or suspending the active ingredient in a vegetable oil or the like, or preparing a microsphere suspension containing a biodegradable polymer compound.

The compound of the present disclosure may also be administered as a concomitant drug in combination with another drug for:
1) complementation and/or enhancement of the prophylactic and/or therapeutic effects of the compound;
2) improvement of kinetics and absorption, and reduction in dosage; and/or
3) attenuation of side effects of the compound.

With respect to a combination drug comprising the compound of the present invention and other drug, may be administered in the form of a blending drug in which both ingredients are blended in one preparation, or may be administered in the form of separate preparations. Administration in these separate preparations includes simultaneous administration and administration by time difference. In addition, administration by time difference may be performed by administering the compound of the present disclosure first and then administering another drug later, or by administering another drug first and then administering the compound of the present disclosure later. The administration methods of the drugs may be the same or different.

The disease for which the above combination drug has a prophylactic and/or therapeutic effect is not particularly limited as long as it is a disease for which the prophylactic and/or therapeutic effect of the compound of the present disclosure are complemented and/or enhanced.

Other drugs for complementing and/or enhancing the prophylactic and/or therapeutic effect of the compound of the present disclosure on Parkinson's disease include, for example, levodopa, carbidopa, pramipexole, ropinirole, rotigotine, apomorphine, zonisamide, droxidopa, cabergoline, pergolide, tolcapone, entacapone, selegiline, rasagiline, safinamide, istradefylline, foslevodopa/foscarbidopa, benserazide, bromocriptine, talipexole, amantadine, trihexyphenidyl, and biperiden.

Other drugs for complementing and/or enhancing the prophylactic and/or therapeutic effect of the compound of the present disclosure on Lewy body disease include, for example, donepezil hydrochloride, rivastigmine, and zonisamide.

Other drugs for complementing and/or enhancing the prophylactic and/or therapeutic effect of the compound of the present disclosure on Gaucher disease include, for example, imiglucerase, velaglucerase alfa, eliglustat, alglucerase, and miglustat.

Any two or more of the other drugs may be administered in combination.

In addition, other drugs that complement and/or enhance the prophylactic and/or therapeutic effect of the compound of the present disclosure include not only those that have been found to date but also those that will be found in the future based on the mechanism described above.

Unless defined otherwise, all technical, scientific terms, and abbreviations used herein have the same meaning as commonly understood by one of ordinary skill in the art to which this invention belongs.

In addition, in the present specification, all contents of all patent documents and non-patent documents or references explicitly cited may be cited herein as part of the present specification.

In one aspect, the present disclosure provides the following embodiments.

[1] A compound represented by formula (I) or a pharmaceutically acceptable salt thereof:

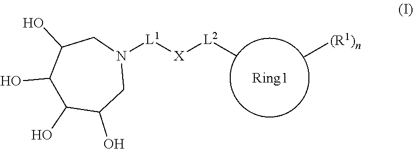

wherein
L¹ represents (1) a C4-7 alkylene, (2) a C4-7 alkenylene, or (3) a C4-7 alkynylene,
L¹ may be substituted with one to five R^{L1}s,
R^{L1} represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, or (10) a hydroxyl group,
a plurality of R^{L}s may be the same or different,
L² represents (1) a C1-3 alkylene, (2) a C2-3 alkenylene or (3) a C2-3 alkynylene,
L² may be substituted with one to four R^{L2}s,
R^{L2} represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, or (10) a hydroxyl group,
a plurality of R^{L2}s may be the same or different,
X represents (1) a bond, (2) —O—, (3) —S—, (4) —SO—, or (5) —SO₂—,
Ring 1 represents (1) a benzene ring (2) a naphthalene ring or (3) a 5- to 10-membered heterocyclyl,
R¹ represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, (10) a C5-7 carbocyclyl, (11) a 5- to 7-membered heterocyclyl, (12) —O—(C5-7 carbocyclyl), or (13) —O-(5- to 7-membered heterocyclyl),
a plurality of R¹s may be the same or different,
the groups (2) to (13) in R¹ may be substituted with one to five R¹⁻¹s,
R¹⁻¹ represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, (10) a C1-6 alkyl sulfonyl group, (11) a cyano group, or (12) a phenyl group,
a plurality of R¹⁻¹s may be the same or different,
the symbol: [symbol]
represents that a substituent is bonded to the α-configuration, β-configuration or a mixture of α- and β-configurations at an appropriate ratio, and
n represents an integer of 0 to 5.

[2] The compound according to the above [1] or a pharmaceutically acceptable salt thereof, wherein L¹ represents a hexylene group.

[3] The compound according to the above [1] or [2] or a pharmaceutically acceptable salt thereof, wherein X represents —O—.

[4] The compound according to any one of the above [1] to [3] or a pharmaceutically acceptable salt thereof, wherein L² represents a methylene group.

[5] The compound according to any one of the above [1] to [4] or a pharmaceutically acceptable salt thereof, wherein Ring 1 represents a ring structure selected from the group consisting of the following ring structures;

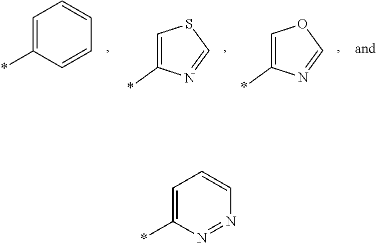 [SMILES: *c1ccccc1 ; *c1cscn1 ; *c1cocn1 ; *c1cccnn1]

wherein the * mark represents a bonding position with L².

[6] The compound according to any one of the above [1] to [5] or a pharmaceutically acceptable salt thereof, wherein R^{L1} represents (1) a halogen atom, or (2) a C1-6 alkyl group.

[7] The compound according to any one of the above [1] to [6] or a pharmaceutically acceptable salt thereof, wherein the compound represented by formula (I) is a compound represented by formula (I-1):

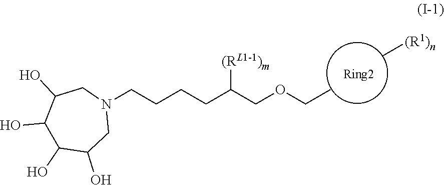

wherein R^{L1-1} represents (1) a halogen atom, or (2) a C1-6 alkyl group,
a plurality of R^{L1-1}s may be the same or different,
m represents an integer of 1 to 2,
Ring 2 represents a ring structure selected from the group consisting of the following ring structures;

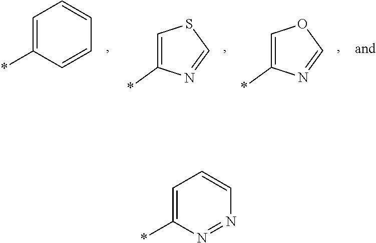 [SMILES: *c1ccccc1 ; *c1cscn1 ; *c1cocn1 ; *c1cccnn1]

wherein the * mark represents a bonding position with methylene, and
other symbols represent the same meaning as the symbols described in the above [1].

[8] The compound according to any one of the above [1] to [7] or a pharmaceutically acceptable salt thereof, wherein the compound represented by formula (I) is a compound selected from the group consisting of:
(1) (3S,4R,5R,6S)-1-{6-[(4-methylbenzyl)oxy]hexyl}-3,4,5,6-azepanetetrol,
(2) (3S,4R,5R,6S)-1-(6-{[2-(3,5-difluorophenyl)-1,3-thiazol-4-yl]methoxy}hexyl)-3,4,5,6-azepanetetrol,
(3) (3S,4R,5R,6S)-1-{(5R)-6-[(3-ethoxy-4-fluorobenzyl)oxy]-5-fluorohexyl}-3,4,5,6-azepanetetrol,
(4) (3S,4R,5R,6S)-1-{(5S)-6-[(3-ethoxy-4-fluorobenzyl)oxy]-5-fluorohexyl}-3,4,5,6-azepanetetrol,
(5) (3S,4R,5R,6S)-1-[(5R)-5-fluoro-6-{[2-(4-fluorophenyl)-1,3-thiazol-4-yl]methoxy}hexyl]-3,4,5,6-azepanetetrol,
(6) (3S,4R,5R,6S)-1-[(5S)-5-fluoro-6-{[2-(4-fluorophenyl)-1,3-thiazol-4-yl]methoxy}hexyl]-3,4,5,6-azepanetetrol,
(7) (3S,4R,5R,6S)-1-[(5R)-6-{[5-(3,5-difluorophenyl)-2-methyl-1,3-oxazol-4-yl]methoxy}-5-fluorohexyl]-3,4,5,6-azepanetetrol,
(8) (3S,4R,5R,6S)-1-[(5S)-6-{[5-(3,5-difluorophenyl)-2-methyl-1,3-oxazol-4-yl]methoxy}-5-fluorohexyl]-3,4,5,6-azepanetetrol,
(9) (3S,4R,5R,6S)-1-{(5S)-6-[(4-ethylbenzyl)oxy]-5-fluorohexyl}-3,4,5,6-azepanetetrol,
(10) (3S,4R,5R,6S)-1-{(5R)-6-[(4-ethylbenzyl)oxy]-5-fluorohexyl}-3,4,5,6-azepanetetrol,
(11) (3S,4R,5R,6S)-1-{(5S)-6-[(4-chlorobenzyl)oxy]-5-fluorohexyl}-3,4,5,6-azepanetetrol,
(12) (3S,4R,5R,6S)-1-{(5R)-6-[(4-chlorobenzyl)oxy]-5-fluorohexyl}-3,4,5,6-azepanetetrol,
(13) (3S,4R,5R,6S)-1-{(5S)-5-fluoro-6-[(4-methylbenzyl)oxy]hexyl}-3,4,5,6-azepanetetrol,
(14) (3S,4R,5R,6S)-1-{(5R)-5-fluoro-6-[(4-methylbenzyl)oxy]hexyl}-3,4,5,6-azepanetetrol,
(15) (3S,4R,5R,6S)-1-[(5S)-6-{[6-(3,5-difluorophenyl)-3-pyridazinyl]methoxy}-5-fluorohexyl]-3,4,5,6-azepanetetrol, and
(16) (3S,4R,5R,6S)-1-[(5R)-6-{[6-(3,5-difluorophenyl)-3-pyridazinyl]methoxy}-5-fluorohexyl]-3,4,5,6-azepanetetrol.

[9] A pharmaceutical composition comprising the compound according to any one of the above [1] to [8] or a pharmaceutically acceptable salt thereof as an active ingredient, and a pharmaceutically acceptable carrier.

[10] The pharmaceutical composition according to the above [9], which is a GBA1 chaperone modulator.

[11] The pharmaceutical composition according to the above [9], which is a GCS inhibitor.

[12] The pharmaceutical composition according to the above [9], which is a GBA1 and GCS dual modulator.

[13] The pharmaceutical composition according to any one of the above [9] to [12], which is an agent for treating and/or preventing a disease associated with GBA1 and/or GCS.

[14] The pharmaceutical composition according to the above [13], wherein the disease associated with GBA1 and/or GCS is selected from the group consisting of Parkinson's disease, Lewy body disease, Gaucher disease, Multiple system atrophy, Fabry disease, Niemann-Pick disease type C, lysosomal storage diseases, Dementia, epilepsy, bipolar disorder, schizophrenia, anxiety disorder, major depression disorder, Sandhoffs disease, Tay-Sachs disease, multiple sclerosis, Huntington's disease, Alzheimer's disease, amyotrophic lateral sclerosis, frontotemporal lobar degeneration, cognitive impairment, learning disabilities, gait disorders, sleeping disorder, neurological disorders, CNS disorders, polycystic kidney disease, type 2 diabetes, cancer, open angle glaucoma, endometriosis, multiple myeloma, virus infection, inflammation, systemic lupus erythematosus, hepatitis, Guillain-Barre Syndrome, diabetic nephropathy, and skin disorder.

[15] The pharmaceutical composition according to the above [13], wherein the disease associated with GBA1 and/or GCS is selected from the group consisting of Parkinson's disease, Lewy body disease, and Gaucher disease.

[16] The pharmaceutical composition according to the above [9], wherein the pharmaceutical composition is administered in combination with one or more agent(s) selected from the group consisting of levodopa, carbidopa, pramipexole, ropinirole, rotigotine, apomorphine, zonisamide, droxidopa, cabergoline, pergolide, tolcapone, entacapone, selegiline, rasagiline, safinamide, istradefylline, foslevodopa/foscarbidopa, benserazide, bromocriptine, talipexole, amantadine, trihexyphenidyl, biperiden, donepezil hydrochloride, imiglucerase, velaglucerase alfa, eliglustat, alglucerase, and miglustat.

[17] An agent for treating and/or preventing a disease associated with GBA1 and/or GCS, comprising the compound according to any one of the above [1] to [8] or a pharmaceutically acceptable salt thereof.

[18] A method for preventing and/or treating a disease associated with GBA1 and/or GCS, the method comprising administering the compound according to any one of the above

or a pharmaceutically acceptable salt thereof, or the pharmaceutical composition according to the above [9] to a patient in need of prevention and/or treatment of a disease associated with GBA1 and/or GCS.

[19] A compound according to any one of the above [1] to [8] or a pharmaceutically acceptable salt thereof, for use in preventing and/or treating a disease associated with GBA1 and/or GCS.

[20] Use of the compound according to any one of the above [1] to [8] or a pharmaceutically acceptable salt thereof, for manufacturing an agent for preventing and/or treating a disease associated with GBA1 and/or GCS.

### Synthesis Examples

The solvent in parentheses shown in the description of the separation by chromatography and TLC indicates an eluting solvent or developing solvent used therefor, and the ratio represents the volume ratio.

The solvent in parentheses shown in the description of NMR indicates the solvent used for the measurement.

The compound names used in the present specification are named using a computer program ACD/Name (registered trademark), which generally performs naming according to the IUPAC nomenclature, or using a Chemdraw Ultra (Version 12.0, manufactured by Cambridge Soft), or according to the IUPAC nomenclature.

LC-MS analysis was performed under the following conditions.

Method A: Reversed-phase LCMS analysis was obtained on a SHIMADZU LC20-MS2020 with ESI source. MS parameters were as follows: Mobile Phase: 1.5 ML/4LTFA in water (solvent A) and 0.75 ML/4LTFA in acetonitrile (solvent B), using the elution gradient 5%-95% (solvent B) over 0.7 minutes and holding at 95% for 0.4 minutes at a flow rate of 1.5 mL/min; Column: Agilent Pursit 5 C18 20*2.0 mm; Wavelength: UV 220 nm, 254 nm; Column temperature: 50° C.; MS ionization: ESI.

Method B: Rersed-phase LCMS analysis was obtained on a SHIMADZU LC20-MS2020 with ESI source. MS parameters were as follows: Mobile Phase: 0.8 mL/4 L NH₃·H₂O in water (solvent A) and acetonitrile (solvent B), sing the elution gradient 10%-80% (solvent B) over 2 minutes and holding at 80% for 0.48 minutes at a flow rate of 1.2 mL/min; Column: Xbridge C18 2.1*50 mm 5 μm; Wavelength: UV 220 nm & 254 nm; Column temperature: 50° C.; MS ionization: ESI.

Method C: Reversed-phase LCMS analysis was obtained on a SHIMADZU LC20-MS2020 with ESI source. MS parameters were as follows: Mobile Phase: 0.8 mL/4 L NH₃·H₂O in water (solvent A) and acetonitrile (solvent B), sing the elution gradient 10%-80% (solvent B) over 6 minutes and holding at 80% for 0.5 minutes at a flow rate of 0.8 mL/min; Column: Xbridge C18 2.1*50 mm 5 μm; Wavelength: UV 220 nm & 254 nm; Column temperature: 50° C.; MS ionization: ESI.

Preparative HPLC was performed under the following conditions or with different solvent gradients and columns.

### Prep-HPLC (HCl):

Mobile Phase: 0.1% HCl in water (solvent A) and acetonitrile (solvent B), using the elution gradient 25%-55% (solvent B) over 8 minutes then holding at 100% for 2 minutes, at a flow rate of 30 mL/min; Column: Phenomenex Synergi C18 150*30 mm*4 μm.

### Prep-HPLC (FA):

Mobile Phase: 0.225% FA in water (solvent A) and acetonitrile (solvent B), using the elution gradient 55%-85% (solvent B) over 8 minutes then holding at 100% for 2 minutes, at a flow rate of 30 mL/min; Column: Welch Xtimate C18 100*40 mm*3 μm.

### Prep-HPLC (Base):

Mobile Phase: 0.05% NH₃H₂O and 10 mM NH₄HCO₃ in water (solvent A) and acetonitrile (solvent B), using the elution gradient 30%-60% (solvent B) over 20 minutes then holding at 100% for 5 minutes, at a flow rate of 30 mL/min; Column: Xtimate C18 150*40 mm*5 μm.

### Prep-HPLC (Neutral):

Mobile Phase: 10 mM NH₄HCO₃ in water (solvent A) and acetonitrile (solvent B), using the elution gradient 35%-65% (solvent B) over 9.5 minutes then holding at 100% for 2 minutes, at a flow rate of 30 ml/min; Column: Phenomenex Gemini C18 75*40 mm*3 μm.

SFC analysis was performed under the following conditions.

Preparative SFC was performed under conditions similar to analytical method or with different Mobile phase, gradients, and columns.

### Method A1:

Waters UPCC with PDA Detector, Column: Cellulose 2 150*4.6 mm I.D., 5 μm, Mobile phase: A: CO₂ B: ethanol (0.05% DEA) Gradient: from 5% to 40% of B in 5 min and hold 40% for 2.5 min, then 5% of B for 2.5 min, Flow rate: 2.5 mL/min, Column temp.: 35° C., ABPR: 1500 psi.

### Method A2:

Waters UPCC with PDA Detector, Column: Cellulose 2 100*4.6 mm I.D., 3 μm, Mobile phase: A: CO₂ B: ethanol (0.05% DEA), Isocratic: 40% B, Flow rate: 2.8 mL/min, Column temp.: 35° C., ABPR: 1500 psi.

### Method A3:

Waters UPCC with PDA Detector, Column: Cellulose 2 150*4.6 mm I.D., 5 μm, Mobile phase: A: CO₂ B: methanol (0.05% DEA), Isocratic: 40% B, Flow rate: 2.5 mL/min, Column temp.: 35° C., ABPR: 1500 psi.

### Method B1:

Waters UPCC with PDA Detector, Column: Chiralpak AD-3 150*4.6 mm I.D., 3 μm, Mobile phase: 50% of ethanol (0.05% DEA) in CO₂, Flow rate: 2.2 mL/min, Column temp.: 35° C., ABPR: 1500 psi.

### Method B2:

Waters UPCC with PDA Detector, Column: Chiralpak AD-3 150*4.6 mm I.D., 3 μm, Mobile phase: A: CO₂ B: ethanol (0.05% DEA), Gradient: from 5% to 40% of B in 4 min and hold 40% for 2 min, then 5% of B for 2 min, Flow rate: 2.5 mL/min, Column temp.: 35° C., ABPR: 1500 psi.

### Method B3:

Waters UPCC with PDA Detector, Column: Chiralpak AD-3 150*4.6 mm I.D., 3 μm, Mobile phase: 40% of methanol (0.05% DEA) in CO₂, Flow rate: 2.5 mL/min, Column temp.: 35° C., ABPR: 1500 psi.

### Method B4:

Agilent 1260 with DAD detector, Column: Chiralpak AD-3 150*4.6 mm I.D., 3 μm, Mobile phase: A: CO₂ B: methanol (0.05% DEA), Gradient: from 5% to 40% of B in 5.5 min and hold 40% for 3 min, then 5% of B for 1.5 min, Flow rate: 2.5 mL/min, Column temp.: 40° C., ABPR: 1500 psi.

### Method B5:

Waters UPCC with PDA Detector, Column: Chiralpak AD-3 150*4.6 mm I.D., 3 μm, Mobile phase: A: CO₂ B: methanol (0.05% DEA), Gradient: from 5% to 40% of B in 5 min and hold 40% for 2.5 min, then 5% of B for 2.5 min, Flow rate: 2.5 mL/min, Column temp.: 35° C., ABPR: 1500 psi.

### Method B6:

Agilent 1260 with DAD detector, Column: Chiralpak AD-3 150*4.6 mm I.D., 3 μm, Mobile phase: A: CO₂ B: methanol (0.05% DEA), Isocratic: 40% B, Flow rate: 2.5 mL/min, Column temp.: 40° C., ABPR: 1500 psi.

### Method B7:

Agilent 1260 with DAD detector, Column: Chiralpak AD-3 50*4.6 mm I.D., 3 μm, Mobile phase: A: CO₂ B: methanol (0.05% DEA), Isocratic: 40% B, Flow rate: 4 mL/min, Column temp.: 35° C., ABPR: 1500 psi.

### Method B8:

Waters UPCC with PDA Detector, Column: Chiralpak AS-3 150*4.6 mm I.D., 3 μm, Mobile phase: 40% of iso-propanol (0.05% DEA) in CO₂, Flow rate: 2.5 mL/min, Column temp.: 35° C., ABPR: 1500 psi.

### Method D1:

Waters UPCC with PDA Detector, Column: Chiralcel OD-3 150*4.6 mm I.D., 3 μm, Mobile phase: A: CO₂ B: ethanol (0.05% DEA), Gradient: from 5% to 40% of B in 4 min and hold 40% for 2 min, then 5% of B for 2 min, Flow rate: 2.5 mL/min, Column temp.: 35° C., ABPR: 1500 psi.

### Method E1:

Waters UPCC with PDA Detector, Column: Chiralcel OJ-3 100*4.6 mm I.D., 3 μm, Mobile phase: A: CO₂ B: ethanol (0.05% DEA), Gradient: from 5% to 40% of B in 4 min and hold 40% for 0.5 min, then 5% of B for 1.5 min, Flow rate: 2.8 mL/min, Column temp.: 35° C., ABPR: 1500 psi.

### Method F1:

Waters UPCC with PDA Detector, Column: Chiralpak IG-3 100*4.6 mm I.D., 3 μm, Mobile phase: 40% of ethanol (0.05% DEA) in CO₂, Flow rate: 2.8 mL/min, Column temp.: 35° C., ABPR: 1500 psi.

### Method F2:

Waters UPCC with PDA Detector, Column: Chiralpak IG-3 50*4.6 mm I.D., 3 μm, Mobile phase: 40% of ethanol (0.05% DEA) in CO₂, Flow rate: 4 mL/min, Column temp.: 35° C., ABPR: 1500 psi.

### Method F3:

Agilent 1260 with DAD detector, Column: Chiralpak IG-3 100*4.6 mm I.D., 3 μm, Mobile phase: A: CO₂ B: ethanol (0.05% DEA), Gradient: from 5% to 40% of B in 4 min and hold 40% for 2.5 min, then 5% of B for 1.5 min, Flow rate: 2.8 mL/min, Column temp.: 35° C., ABPR: 1500 psi.

### Method F4:

Waters UPCC with PDA Detector, Column: Chiralpak IG-3 50*4.6 mm I.D., 3 μm, Mobile phase: A: CO₂ B: methanol (0.05% DEA), Gradient: from 5% to 40% of B in 2 min and hold 40% for 1.2 min, then 5% of B for 0.8 min, Flow rate: 4 mL/min, Column temp.: 35° C., ABPR: 1500 psi.

### Method F5:

Agilent 1260 with DAD detector, Column: Chiralpak IG-3 100*4.6 mm I.D., 3 μm, Mobile phase: A: CO₂ B: methanol (0.05% DEA), Gradient: from 5% to 40% of B in 4 min and hold 40% for 3 min, then 5% of B for 1 min, Flow rate: 2.5 mL/min, Column temp.: 40° C., ABPR: 1500 psi.

### Method F6:

Waters UPCC with PDA Detector Column: Chiralpak IG-3 50*4.6 mm I.D., 3 μm, Mobile phase: A: CO₂ B: iso-propanol (0.05% DEA), Isocratic: 40% B, Flow rate: 4 mL/min, Column temp.: 35° C., ABPR: 1500 psi.

### Method G1:

Agilent 1260 with DAD detector, Column: Cellulose-4 100*4.6 mm I.D., 3 μm, Mobile phase: A: CO₂ B: ethanol (0.05% DEA), Gradient: from 5% to 40% of B in 4 min and hold 40%, for 2.5 min, then 5% of B for 1.5 min, Flow rate: 2.8 mL/min Column temp.: 40° C., Back pressure: 100 bar.

### Method G2:

Agilent 1260 with DAD detector, Column: Cellulose-4 100*4.6 mm I.D., 3 μm, Mobile phase: A: CO₂ B: ethanol (0.05% DEA), Isocratic: 40% B, Flow rate: 2.8 mL/min Column temp.: 40° C., Back pressure: 100 bar.

### Method H₁:

Agilent 1260 with DAD detector, Column: ChiralPak AY-3 150*4.6 mm I.D., 3 μm, Mobile phase: A: CO₂ B: ethanol (0.05% DEA), Gradient: from 5% to 40% of B in 5.5 min and hold 40%, for 3 min, then 5% of B for 1.5 min, Flow rate: 2.5 mL/min, Column temp.: 40° C., ABPR: 100 bar.

### Method J1:

Agilent 1260 with DAD detector, Column: Chiralcel OJ-H 150*4.6 mm I.D., 5 μm, Mobile phase: A: CO₂ B: ethanol (0.05% DEA), Gradient: from 5% to 40% of B in 4.5 min then 5% of B for 1.5 min, Flow rate: 2.5 mL/min, Column temp.: 40° C., ABPR: 1500 psi.

Abbreviations which have been used in the descriptions of following examples are: FA is formic acid; TFA is trifluoromethyl acetic acid; TEA is triethylamine; DIEA is N,N-diisopropylethylamine; DIAD is diisopropyl diazene-1,2-dicarboxylate; DAST is diethylaminosulfur trifluoride; DCM is dichloromethane; DMF is N,N-dimethylformamide; DMSO is dimethyl sulfoxide; EtOAc is ethyl acetate; PE is Petroleum Ether; THF is tetrahydrofuran; DMP is Dess-Martin Periodinane; Pd(dppf)Cl₂ is [1,1′-Bis(diphenylphosphino)ferrocene]dichloropalladium(II); TBAI is tetrabutylammonium iodide; TBAF is tetrabutylammonium fluoride; DMAP is N,N-dimethyl-4-aminopyridine; IPA is isopropanol; MTBE is methyl tert-butyl ether; NFSI is N-fluorobis(phenylsulfonyl)amine; PBSF is perfluoro-1-butanesulfonyl fluoride; DBU is 1,8-diazabicyclo[5.4.0]undec-7-ene; DMA is N,N-Dimethylacetamide; TBAB is tetrabutylammonium bromide; mCPBA is m-chloroperoxybenzoic acid.

### Reference Example 1: 6-iodohexyl 4-methylbenzyl Ether

To a mixture of (4-methylphenyl)methanol (CAS No. 589-18-4, 0.16 ml) and t-BuOK (207 mg) in THF (5 ml) was added 1,6-diiodohexane (CAS No. 629-09-4, 0.61 mL) at 25° C. The reaction mixture was stirred at 40° C. for 14 h. The mixture was diluted with DCM and concentrated to dryness. The crude product was purified by flash column with silica gel (PE to 15% EtOAC in PE) to give the title compound (103 mg).

¹H NMR (CDCl₃) δ: 7.29, 7.24, 4.48, 3.54-3.44, 3.19, 2.49-2.33, 1.94-1.82, 1.66-1.57, 1.53-1.37.

### Example 1: (3S,4R,5R,6S)-1-{6-[(4-methylbenzyl)oxy]hexyl}-3,4,5,6-azepanetetrol

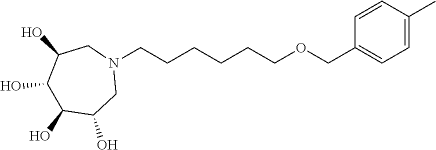 [SMILES: Cc1ccc(COCCCCCCN2C[C@H](O)[C@@H](O)[C@H](O)[C@@H](O)C2)cc1]

To a mixture of KI (57 mg) in DMF (2 mL) was added the compound prepared in Reference Example 1 (103 mg), (3S,4R,5R,6S)-azepane-3,4,5,6-tetrol hydrochloride salt (CAS No. 170210-58-9, 93 mg) and DIEA (0.26 mL) at 25° C. The reaction was stirred at 80° C. for 16 h. The residue was purified by prep-HPLC (base) to give the title compound (67 mg).

LCMS: retention time 1.622 min, (Method B); MS (ESI, Pos.): m/z 368.2 (M+H)+; ¹H NMR (CDCl₃) δ: 7.26, 7.18, 4.45, 3.72-3.65, 3.60-3.53, 3.44, 2.89, 2.60, 2.53, 2.34, 1.63-1.55, 1.52-1.43, 1.41-1.34, 1.33-1.26.

### Example 2 to 60

Example 2 to 60 were synthesized according to procedures analogous to Reference Example 1 and Example 1 using the corresponding alcohol compound instead of (4-methylphenyl)methanol.

### Example 2: (3S,4R,5R,6S)-1-[6-(4-biphenylylmethoxy)hexyl]-3,4,5,6-azepanetetrol

LCMS: retention time 1.835 min, (Method B); MS (ESI, Pos.): m/z 430.3 (M+H)+;

¹H NMR (CD₃OD) δ: 7.62-7.54, 7.48-7.36, 7.37-7.30, 4.54, 3.60-3.50, 3.42-3.36, 2.80, 2.56-2.49, 1.69-1.60, 1.50-1.31.

### Example 3: (3S,4R,5R,6S)-1-{6-[(3-phenyl-2-propyn-1-yl)oxy]hexyl}-3,4,5,6-azepanetetrol Hydrochloride (1:1)

LCMS: retention time 0.67 min, (Method A); MS (ESI, Pos.): m/z 378.1 (M+H)+;

¹H NMR (D₂O) δ: 7.44, 7.39-7.29, 4.33, 4.10-3.95, 3.76-3.44, 3.39-3.07, 1.72-1.52, 1.38-1.27.

### Example 4: (3S,4R,5R,6S)-1-(6-{[2-(4-fluorophenyl)-1,3-thiazol-4-yl]methoxy}hexyl)-3,4,5,6-azepanetetrol

LCMS: retention time 0.692 min, (Method A); MS (ESI, Pos.): m/z 455.2 (M+H)+;

¹H NMR (CDCl₃) δ: 7.95-7.89, 7.20, 7.20-7.08, 4.65, 4.48-3.75, 3.66, 3.66-3.51, 2.84-2.81, 2.59-2.46, 1.66-1.60, 1.46-1.26.

### Example 5: (3S,4R,5R,6S)-1-{6-[(2-phenyl-1,3-thiazol-4-yl)methoxy]hexyl}-3,4,5,6-azepanetetrol Hydrochloride (1:1)

LCMS: retention time 0.688 min, (Method A); MS (ESI, Pos.): m/z 437.1 (M+H)+;

¹H NMR (CD₃OD) δ: 8.05-7.95, 7.54-7.52, 4.68, 4.21-4.04, 3.86, 3.69-3.55, 3.44-3.36, 3.29-3.14, 1.90-1.65, 1.56-1.40.

### Example 6: (3S,4R,5R,6S)-1-[6-({2-[4-(trifluoromethyl)phenyl]-1,3-thiazol-4-yl}methoxy)hexyl]-3,4,5,6-azepanetetrol Hydrochloride (1:1)

LCMS: retention time 0.751 min, (Method A); MS (ESI, Pos.): m/z 505.1 (M+H)+;

¹H NMR (CD₃OD) δ: 8.17, 7.82, 7.59, 4.71, 4.22-4.08, 3.88, 3.70-3.59, 3.48-3.38, 3.31-3.16, 1.91-1.67, 1.59-1.43.

### Example 7: (3S,4R,5R,6S)-1-[6-({2-[3-(trifluoromethyl)phenyl]-1,3-thiazol-4-yl}methoxy)hexyl]-3,4,5,6-azepanetetrol Hydrochloride (1:1)

LCMS: retention time 0.75 min, (Method A); MS (ESI, Pos.): m/z 505.1 (M+H)+;

¹H NMR (CD₃OD) δ: 8.29, 8.21, 7.83, 7.74, 7.58, 4.71, 4.21-4.05, 3.90-3.85, 3.68-3.55, 3.46-3.35, 3.29-3.14, 1.87-1.65, 1.58-1.38.

### Example 8: (3S,4R,5R,6S)-1-(6-{[2-(3-fluorophenyl)-1,3-thiazol-4-yl]methoxy}hexyl)-3,4,5,6-azepanetetrol Hydrochloride (1:1)

LCMS: retention time 0.698 min, (Method A); MS (ESI, Pos.): m/z 455.1 (M+H)+;

¹H NMR (CD₃OD) δ: 7.86-7.71, 7.61-7.48, 7.30-7.20, 4.68, 4.19-4.02, 3.84, 3.65-3.57, 3.45-3.34, 3.27-3.14, 1.92-1.63, 1.57-1.37.

### Example 9: (3S,4R,5R,6S)-1-(6-{[2-(2-methylphenyl)-1,3-thiazol-4-yl]methoxy}hexyl)-3,4,5,6-azepanetetrol Hydrochloride (1:1)

LCMS: retention time 0.701 min, (Method A); MS (ESI, Pos.): m/z 451.1 (M+H)+;

¹H NMR (CD₃OD) δ: 7.89, 7.65, 7.57-7.33, 4.72, 4.19-4.03, 3.83, 3.67-3.53, 3.43-3.32, 3.27-3.11, 2.51, 1.89-1.63, 1.56-1.37.

### Example 10: (3S,4R,5R,6S)-1-(6-{[2-(2-fluorophenyl)-1,3-thiazol-4-yl]methoxy}hexyl)-3,4,5,6-azepanetetrol Hydrochloride (1:1)

LCMS: retention time 0.704 min, (Method A); MS (ESI, Pos.): m/z 455.1 (M+H)+;

¹H NMR (CD₃OD) δ: 8.25, 7.62, 7.58-7.48, 7.41-7.27, 4.71, 4.22-4.06, 3.86, 3.68-3.56, 3.46-3.36, 3.27-3.14, 1.89-1.65, 1.57-1.38.

### Example 11: (3S,4R,5R,6S)-1-{6-[(5-phenyl-2-pyrazinyl)methoxy]hexyl}-3,4,5,6-azepanetetrol

LCMS: retention time 0.676 min, (Method A); MS (ESI, Pos.): m/z 432.1 (M+H)+;

¹H NMR (CD₃OD) δ: 9.05, 8.75, 8.04, 7.64-7.46, 4.72, 3.68-3.57, 3.44-3.37, 2.81, 2.61-2.45, 1.73-1.63, 1.55-1.28.

### Example 12: (3S,4R,5R,6S)-1-(6-{[2-(4-fluorophenyl)-1,3-thiazol-5-yl]methoxy}hexyl)-3,4,5,6-azepanetetrol Hydrochloride (1:1)

LCMS: retention time 0.739 min, (Method A); MS (ESI, Pos.): m/z 455.2 (M+H)+;

¹H NMR (D₂O) δ: 7.92-7.86, 7.27, 4.78, 4.08-4.06, 3.72, 3.60, 3.33, 3.20-3.15, 1.70-1.56, 1.35-1.29.

### Example 13: (3S,4R,5R,6S)-1-(6-{[4-(4-fluorophenyl)-1,3-thiazol-2-yl]methoxy}hexyl)-3,4,5,6-azepanetetrol

LCMS: retention time 0.755 min, (Method A); MS (ESI, Pos.): m/z 455.2 (M+H)+;

¹H NMR (CD₃OD) δ: 7.94-7.89, 7.75, 7.15, 4.82, 3.65, 3.58-3.56, 3.40-3.37, 2.83, 2.57-2.51, 1.70-1.64, 1.51-1.35.

### Example 14: (3S,4R,5R,6S)-1-(6-{[2-(4-fluorophenyl)-1,3-oxazol-4-yl]methoxy}hexyl)-3,4,5,6-azepanetetrol Hydrochloride (1:1)

LCMS: retention time 0.731 min, (Method A); MS (ESI, Pos.): m/z 439.2 (M+H)+;

¹H NMR (D₂O) δ: 7.96-7.88, 7.22, 4.45, 4.07-4.05, 3.74, 3.60-3.50, 3.42-3.23, 3.17-3.12, 1.70-1.53, 1.34-1.30.

### Example 15: (3S,4R,5R,6S)-1-(6-{[2-(4-fluorophenyl)-5-methyl-1,3-thiazol-4-yl]methoxy}hexyl)-3,4,5,6-azepanetetrol Hydrochloride (1:1)

LCMS: retention time 0.712 min, (Method A); MS (ESI, Pos.): m/z 469 (M+H)+;

¹H NMR (D₂O) δ: 7.88-7.73, 7.22, 4.56, 4.08-3.92, 3.70, 3.60-3.50, 3.41-2.99, 2.44, 1.75-1.43, 1.38-1.14.

### Example 16: (3S,4R,5R,6S)-1-{6-[(3′-fluoro-4-biphenylyl)methoxy]hexyl}-3,4,5,6-azepanetetrol

LCMS: retention time 0.744 min, (Method A); MS (ESI, Pos.): m/z 448.1 (M+H)+;

¹H NMR (CD₃CN) δ: 7.63, 7.55-7.36, 7.20-7.05, 4.51, 3.86-3.30, 2.78, 2.50, 1.68-1.51, 1.52-1.24.

### Example 17: (3S,4R,5R,6S)-1-(6-{[3′-(trifluoromethyl)-4-biphenylyl]methoxy}hexyl)-3,4,5,6-azepanetetrol

LCMS: retention time 0.767 min, (Method A); MS (ESI, Pos.): m/z 498.1 (M+H)+;

¹H NMR (CDCl₃) δ: 7.81, 7.73, 7.61-7.48, 7.41, 4.52, 3.70, 3.58, 3.48, 2.88, 2.66-2.46, 1.71-1.18.

### Example 18: (3S,4R,5R,6S)-1-(6-{[4′-(trifluoromethyl)-4-biphenylyl]methoxy}hexyl)-3,4,5,6-azepanetetrol

LCMS: retention time 0.785 min, (Method A); MS (ESI, Pos.): m/z 498.1 (M+H)+;

¹H NMR (CDCl₃) δ: 7.70-7.60, 7.55, 7.40, 4.51, 3.84, 3.69, 3.47, 2.99, 2.84-2.58, 1.69-1.46, 1.43-1.19.

### Example 19: (3S,4R,5R,6S)-1-(6-{[2-(4-chlorophenyl)-1,3-thiazol-4-yl]methoxy}hexyl)-3,4,5,6-azepanetetrol

LCMS: retention time 0.72 min, (Method A); MS (ESI, Pos.): m/z 471 (M+H)+;

¹H NMR (CD₃OD) δ: 7.95, 7.56-7.48, 4.66, 3.64-3.55, 3.46-3.38, 2.82, 2.60-2.45, 1.72-1.61, 1.56-1.29.

### Example 20: (3S,4R,5R,6S)-1-(6-{[2-(3,4-difluorophenyl)-1,3-thiazol-4-yl]methoxy}hexyl)-3,4,5,6-azepanetetrol

LCMS: retention time 0.707 min, (Method A); MS (ESI, Pos.): m/z 473.1 (M+H)+;

¹H NMR (CD₃OD) δ: 7.95-7.82, 7.80-7.69, 7.49, 7.45-7.34, 4.63, 3.65-3.52, 3.43-3.36, 2.81, 2.57-2.49, 1.71-1.59, 1.56-1.27.

### Example 21: (3S,4R,5R,6S)-1-(6-{[2-(3,5-difluorophenyl)-1,3-thiazol-4-yl]methoxy}hexyl)-3,4,5,6-azepanetetrol

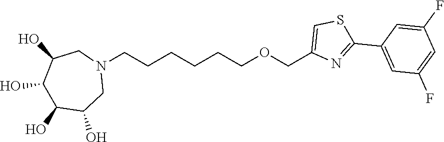 [SMILES: O[C@H]1[C@H](O)[C@@H](O)CN(CCCCCCOCc2csc(-c3cc(F)cc(F)c3)n2)C[C@@H]1O]

LCMS: retention time 0.709 min, (Method A); MS (ESI, Pos.): m/z 473.1 (M+H)+;

¹H NMR (CD₃OD) δ: 7.69-7.50, 7.15-7.02, 4.67, 3.68-3.53, 3.46-3.37, 2.90-2.76, 2.58-2.44, 1.70-1.60, 1.56-1.30.

### Example 22: (3S,4R,5R,6S)-1-(6-{[2-(4-methylphenyl)-1,3-thiazol-4-yl]methoxy}hexyl)-3,4,5,6-azepanetetrol

LCMS: retention time 0.71 min, (Method A); MS (ESI, Pos.): m/z 451.1 (M+H)+;

¹H NMR (CD₃OD) δ: 7.84, 7.42, 7.31, 4.64, 3.70-3.55, 3.44-3.38, 2.82, 2.65-2.47, 2.41, 1.72-1.60, 1.56-1.29.

### Example 23: (3S,4R,5R,6S)-1-(6-{[2-(4-methoxyphenyl)-1,3-thiazol-4-yl]methoxy}hexyl)-3,4,5,6-azepanetetrol

LCMS: retention time 0.749 min, (Method A); MS (ESI, Pos.): m/z 467.1 (M+H)+;

¹H NMR (CD₃OD) δ: 7.91, 7.39, 7.06, 4.64, 3.89, 3.68-3.56, 3.44, 2.86, 2.74-2.48, 1.72-1.60, 1.59-1.29.

### Example 24: (3S,4R,5R,6S)-1-(6-{[2-(3,4,5-trifluorophenyl)-1,3-thiazol-4-yl]methoxy}hexyl)-3,4,5,6-azepanetetrol

LCMS: retention time 0.724 min, (Method A); MS (ESI, Pos.): m/z 491 (M+H)+;

¹H NMR (CD₃OD) δ: 7.81-7.70, 7.54, 4.64, 3.72, 3.59, 3.48, 2.94, 2.84-2.60, 1.70-1.51, 1.49-1.30.

### Example 25: (3S,4R,5R,6S)-1-(6-{[2-(3-chloro-4-fluorophenyl)-1,3-thiazol-4-yl]methoxy}hexyl)-3,4,5,6-azepanetetrol Hydrochloride (1:1)

LCMS: retention time 0.725 min, (Method A); MS (ESI, Pos.): m/z 489 (M+H)+;

¹H NMR (CD₃OD) δ: 8.13, 7.99-7.88, 7.53, 7.40, 4.66, 4.22-4.04, 3.85, 3.69-3.52, 3.47-3.35, 3.29-3.14, 1.91-1.65, 1.55-1.37.

### Example 26: (3S,4R,5R,6S)-1-[6-({2-[4-(trifluoromethoxy)phenyl]-1,3-thiazol-4-yl}methoxy)hexyl]-3,4,5,6-azepanetetrol

LCMS: retention time 0.746 min, (Method A); MS (ESI, Pos.): m/z 521.1 (M+H)+;

¹H NMR (CD₃OD) δ: 8.07, 7.50, 7.40, 4.65, 3.66-3.51, 3.40-3.36, 2.78, 2.56-2.50, 1.72-1.58, 1.54-1.26.

### Example 27: (3S,4R,5R,6S)-1-(6-{[2-(3-methoxyphenyl)-1,3-thiazol-4-yl]methoxy}hexyl)-3,4,5,6-azepanetetrol

LCMS: retention time 0.696 min, (Method A); MS (ESI, Pos.): m/z 467.1 (M+H)+;

¹H NMR (CD₃OD) δ: 7.54-7.46, 7.39, 7.04, 4.58, 3.85, 3.61-3.55, 3.46, 2.78, 2.50-2.42, 1.63-1.54, 1.47-1.24.

### Example 28: (3S,4R,5R,6S)-1-[6-(3-biphenylylmethoxy)hexyl]-3,4,5,6-azepanetetrol

LCMS: retention time 0.726 min, (Method A); MS (ESI, Pos.): m/z 430.1 (M+H)+;

¹H NMR (CD₃OD) δ: 7.65-7.57, 7.54, 7.48-7.39, 7.38-7.28, 4.57, 3.61-3.55, 3.53, 3.41-3.36, 2.80, 2.58-2.46, 1.71-1.58, 1.54-1.29.

### Example 29: (3S,4R,5R,6S)-1-(6-{[4-(1,3-thiazol-4-yl)benzyl]oxy}hexyl)-3,4,5,6-azepanetetrol Hydrochloride (1:1)

LCMS: retention time 0.669 min, (Method A); MS (ESI, Pos.): m/z 437.1 (M+H)+;

¹H NMR (D₂O) δ: 9.07, 7.88-7.71, 7.40, 4.48, 4.07-3.92, 3.67, 3.60-3.39, 3.37-2.97, 1.72-1.40, 1.35-1.12.

### Example 30: (3S,4R,5R,6S)-1-[6-({2-[3-(trifluoromethoxy)phenyl]-1,3-thiazol-4-yl}methoxy)hexyl]-3,4,5,6-azepanetetrol

LCMS: retention time 0.721 min, (Method A); MS (ESI, Pos.): m/z 521.1 (M+H)+;

¹H NMR (CD₃OD) δ: 7.96-7.86, 7.58, 7.52, 7.38, 4.65, 3.65-3.55, 3.43-3.36, 2.82, 2.66-2.45, 1.69-1.60, 1.55-1.46, 1.46-1.38, 1.37-1.29.

### Example 31: 3-{4-[({6-[(3S,4R,5R,6S)-3,4,5,6-tetrahydroxy-1-azepanyl]hexyl}oxy)methyl]-1,3-thiazol-2-yl}benzonitrile

LCMS: retention time 0.678 min, (Method A); MS (ESI, Pos.): m/z 462.1 (M+H)+;

¹H NMR (CD₃OD) δ: 8.34, 8.26, 7.83, 7.69, 7.57, 4.67, 3.65-3.53, 3.43-3.36, 2.80, 2.60-2.46, 1.72-1.61, 1.56-1.25.

### Example 32: 4-{4-[({6-[(3S,4R,5R,6S)-3,4,5,6-tetrahydroxy-1-azepanyl]hexyl}oxy)methyl]-1,3-thiazol-2-yl}benzonitrile

LCMS: retention time 0.673 min, (Method A); MS (ESI, Pos.): m/z 462.2 (M+H)+;

¹H NMR (CD₃OD) δ: 8.15, 7.86, 7.60, 4.68, 3.67-3.52, 3.46-3.36, 2.81, 2.64-2.45, 1.71-1.60, 1.57-1.26.

### Example 33: (3S,4R,5R,6S)-1-(6-{[2-(4-fluoro-3-methoxyphenyl)-1,3-thiazol-4-yl]methoxy}hexyl)-3,4,5,6-azepanetetrol

LCMS: retention time 0.703 min, (Method A); MS (ESI, Pos.): m/z 485.1 (M+H)+;

¹H NMR (CD₃OD) δ: 7.72, 7.51-7.42, 7.19, 4.63, 3.96, 3.65-3.55, 3.44-3.37, 2.83, 2.64-2.49, 1.71-1.58, 1.56-1.27.

### Example 34: (3S,4R,5R,6S)-1-[6-({2-[4-(methylsulfonyl)phenyl]-1,3-thiazol-4-yl}methoxy)hexyl]-3,4,5,6-azepanetetrol

LCMS: retention time 0.656 min, (Method A); MS (ESI, Pos.): m/z 515 (M+H)+;

¹H NMR (CD₃OD) δ: 8.25, 8.09, 7.62, 4.70, 3.66-3.55, 3.43-3.38, 3.20, 2.82, 2.62-2.47, 1.72-1.61, 1.56-1.30.

### Example 35: (3S,4R,5R,6S)-1-(6-{[6-(3-fluoro-5-methoxyphenyl)-3-pyridinyl]methoxy}hexyl)-3,4,5,6-azepanetetrol Hydrochloride (1:1)

LCMS: retention time 1.031 min, (Method D); MS (ESI, Pos.): m/z 479.2 (M+H)+;

¹H NMR (D₂O) δ: 8.74, 8.59, 8.26, 7.32-7.15, 7.05, 4.10, 3.89, 3.86-3.02, 1.84-1.57, 1.46-1.31.

### Example 36: (3S,4R,5R,6S)-1-(6-{[5-(3-fluoro-5-methoxyphenyl)-2-pyridinyl]methoxy}hexyl)-3,4,5,6-azepanetetrol

LCMS: retention time 0.665 min, (Method A); MS (ESI, Pos.): m/z 479.1 (M+H)+;

¹H NMR (CD₃OD) δ: 8.73, 8.10, 7.61, 7.12-6.94, 6.76, 4.64, 3.87, 3.65-3.52, 3.43-3.35, 2.81, 2.62-2.44, 1.74-1.61, 1.58-1.40, 1.40-1.30.

### Example 37: (3S,4R,5R,6S)-1-(6-{[2-(3-chloro-5-methoxyphenyl)-1,3-thiazol-4-yl]methoxy}hexyl)-3,4,5,6-azepanetetrol

LCMS: retention time 1.808 min, (Method B); MS (ESI, Pos.): m/z 501.2 (M+H)+;

¹H NMR (CD₃OD) δ: 7.60-7.50, 7.49, 7.09, 4.67, 3.91, 3.70-3.55, 3.43, 2.85, 2.64-2.53, 1.71-1.65, 1.55-1.36.

### Example 38: (3S,4R,5R,6S)-1-{6-[(2-cyclohexyl-1,3-thiazol-4-yl)methoxy]hexyl}-3,4,5,6-azepanetetrol

LCMS: retention time 0.715 min, (Method A); MS (ESI, Pos.): m/z 443 (M+H)+;

¹H NMR (CD₃OD) δ: 7.28, 4.53, 3.62-3.52, 3.44-3.37, 3.05-2.95, 2.82, 2.60-2.48, 2.16-2.09, 1.92-1.84, 1.82-1.75, 1.67-1.20.

### Example 39: (3S,4R,5R,6S)-1-{6-[(2-propyl-1,3-thiazol-4-yl)methoxy]hexyl}-3,4,5,6-azepanetetrol

LCMS: retention time 0.655 min, (Method A); MS (ESI, Pos.): m/z 403 (M+H)+;

¹H NMR (CD₃OD) δ: 7.32, 4.57, 3.68-3.53, 3.45-3.39, 3.01, 2.84, 2.67-2.50, 1.96-1.75, 1.69-1.60, 1.58-1.30, 1.04.

### Example 40: (3S,4R,5R,6S)-1-(6-{[2-(2,4-difluorophenyl)-1,3-thiazol-4-yl]methoxy}hexyl)-3,4,5,6-azepanetetrol

LCMS: retention time 0.723 min, (Method A); MS (ESI, Pos.): m/z 473.1 (M+H)+;

¹H NMR (CD₃OD) δ: 8.28, 7.56, 7.22-7.08, 4.67, 3.61-3.54, 3.42-3.35, 2.80, 2.58-2.48, 1.69-1.60, 1.55-1.46, 1.45-1.37, 1.36-1.29.

### Example 41: (3S,4R,5R,6S)-1-(6-{[2-(2,5-difluorophenyl)-1,3-thiazol-4-yl]methoxy}hexyl)-3,4,5,6-azepanetetrol

LCMS: retention time 0.764 min, (Method A); MS (ESI, Pos.): m/z 473.2 (M+H)+;

¹H NMR (CD₃OD) δ: 8.02-7.95, 7.63, 7.40-7.35, 7.27-7.20, 4.69, 3.70-3.57, 3.42, 2.92-2.80, 2.69-2.51, 1.70-1.61, 1.66-1.55, 1.48-1.41, 1.39-1.32.

### Example 42: (3S,4R,5R,6S)-1-(6-{[2-(2,6-difluorophenyl)-1,3-thiazol-4-yl]methoxy}hexyl)-3,4,5,6-azepanetetrol Hydrochloride (1:1)

LCMS: retention time 0.689 min, (Method A); MS (ESI, Pos.): m/z 473.1 (M+H)+;

¹H NMR (D₂O) δ: 7.76, 7.57-7.51, 7.20-7.15, 4.69, 4.18-4.02, 3.88-3.53, 3.49-3.10, 1.84-1.53, 1.43-1.24.

### Example 43: (3S,4R,5R,6S)-1-(6-{[2-(2,3,4-trifluorophenyl)-1,3-thiazol-4-yl]methoxy}hexyl)-3,4,5,6-azepanetetrol

LCMS: retention time 0.78 min, (Method A); MS (ESI, Pos.): m/z 491.2 (M+H)+;

¹H NMR (CD₃OD) δ: 8.10-8.00, 7.63, 7.35-7.24, 4.68, 3.63-3.51, 3.43-3.34, 2.79, 2.59-2.44, 1.70-1.59, 1.55-1.27.

### Example 44: (3S,4R,5R,6S)-1-(6-{[2-(2,4,6-trifluorophenyl)-1,3-thiazol-4-yl]methoxy}hexyl)-3,4,5,6-azepanetetrol Hydrochloride (1:1)

LCMS: retention time 0.749 min, (Method A); MS (ESI, Pos.): m/z 491.2 (M+H)+;

¹H NMR (CD₃OD) δ: 7.71, 7.15-7.04, 4.68, 3.65-3.53, 3.45-3.37, 2.84, 2.63-2.50, 1.66-1.33.

### Example 45: (3S,4R,5R,6S)-1-[6-({2-[(E)-2-phenylvinyl]-1,3-thiazol-4-yl}methoxy)hexyl]-3,4,5,6-azepanetetrol

LCMS: retention time 1.706 min, (Method B); MS (ESI, Pos.): m/z 463.2 (M+H)+;

¹H NMR (CD₃OD) δ: 7.64, 7.55-7.48, 7.46-7.35, 4.62, 3.63-3.56, 3.44-3.40, 2.83, 2.60-2.51, 1.70-1.63, 1.57-1.33.

### Example 46: (3S,4R,5R,6S)-1-[6-(2-naphthylmethoxy)hexyl]-3,4,5,6-azepanetetrol

LCMS: retention time 1.729 min, (Method B); MS (ESI, Pos.): m/z 404.3 (M+H)+;

¹H NMR (CD₃OD) δ: 7.90-7.79, 7.62-7.35, 4.67, 3.60-3.53, 3.42-3.37, 2.81, 2.58-2.47, 1.70-1.62, 1.54-1.41, 1.37-1.30.

### Example 47: (3S,4R,5R,6S)-1-[6-(2-biphenylylmethoxy)hexyl]-3,4,5,6-azepanetetrol

LCMS: retention time 1.828 min, (Method B); MS (ESI, Pos.): m/z 430.3 (M+H)+;

¹H NMR (DMSO-D₆) δ: 7.54-7.51, 7.51-7.33, 7.30-7.23, 4.36, 3.64-3.52, 3.44-3.34, 2.83, 2.63-2.43, 1.59-1.43, 1.38-1.26.

### Example 48: (3S,4R,5R,6S)-1-{6-[(3′,5′-difluoro-2-biphenylyl)methoxy]hexyl}-3,4,5,6-azepanetetrol

LCMS: retention time 1.89 min, (Method B); MS (ESI, Pos.): m/z 466.3 (M+H)+;

¹H NMR (CD₃OD) δ: 7.56-7.25, 7.22-6.91, 4.35, 3.63-3.50, 3.45-3.36, 2.80, 2.56-2.49, 1.61-1.44, 1.41-1.26.

### Example 49: (3S,4R,5R,6S)-1-{6-[(3,4-dipropylbenzyl)oxy]hexyl}-3,4,5,6-azepanetetrol

LCMS: retention time 2.148 min, (Method B); MS (ESI, Pos.): m/z 438.4 (M+H)+;

¹H NMR (CD₃OD) δ: 7.15-7.01, 4.43, 3.60-3.53, 3.47, 3.40-3.35, 2.79, 2.63-2.46, 1.64-1.55, 1.52-1.45, 1.42-1.36, 1.34-1.28, 1.02-0.97.

### Example 50: Formic Acid—(3S,4R,5R,6S)-1-(6-{[2-(3-methylbutyl)benzyl]oxy}hexyl)-3,4,5,6-azepanetetrol (1:1)

LCMS: retention time 0.756 min, (Method A); MS (ESI, Pos.): m/z 424.2 (M+H)+;

¹H NMR (DMSO-D₆) δ: 8.18, 7.37-7.08, 4.44, 3.31-2.99, 2.66, 2.62-2.57, 2.57-2.54, 2.46-2.39, 1.64-1.47, 1.44-1.22, 0.93.

### Example 51: (3S,4R,5R,6S)-1-{6-[(4-propylbenzyl)oxy]hexyl}-3,4,5,6-azepanetetrol

LCMS: retention time 1.854 min, (Method B); MS (ESI, Pos.): m/z 396.4 (M+H)+;

¹H NMR (CD₃OD) δ: 7.24, 7.17, 4.45, 3.60, 3.48, 3.44-3.37, 2.84, 2.64-2.49, 1.67-1.56, 1.54-1.46, 1.43-1.29, 0.93.

### Example 52: (3S,4R,5R,6S)-1-(6-{[4-(1-cyclohexen-1-yl)benzyl]oxy}hexyl)-3,4,5,6-azepanetetrol

LCMS: retention time 0.781 min, (Method A); MS (ESI, Pos.): m/z 456.2 (M+Na)+;

¹H NMR (CDCl₃) δ: 7.35, 7.26, 6.16-6.05, 4.46, 3.75, 3.60, 3.44, 2.93, 2.76-2.63, 2.63-2.52, 2.41-2.37, 2.22-2.17, 1.81-1.73, 1.69-1.56, 1.54-1.45, 1.38-1.25.

### Example 53: (3S,4R,5R,6S)-1-(6-{[3-(1-cyclohexen-1-yl)benzyl]oxy}hexyl)-3,4,5,6-azepanetetrol

LCMS: retention time 1.996 min, (Method B); MS (ESI, Pos.): m/z 434.4 (M+H)+;

¹H NMR (CD₃OD) δ: 7.36-7.23, 7.17, 6.11, 4.48, 3.58, 3.49, 3.43-3.35, 2.81, 2.61-2.47, 2.41, 2.28-2.17, 1.86-1.75, 1.74-1.57, 1.55-1.45, 1.43-1.28.

### Example 54: (3S,4R,5R,6S)-1-{6-[(3-cyclohexylbenzyl)oxy]hexyl}-3,4,5,6-azepanetetrol

LCMS: retention time 0.865 min, (Method A); MS (ESI, Pos.): m/z 436.3 (M+H)+;

¹H NMR (CD₃OD) δ: 7.30-7.06, 4.46, 3.65-3.52, 3.52-3.44, 3.43-3.35, 2.83, 2.61-2.44, 1.92-1.80, 1.80-1.71, 1.65-1.56, 1.54-1.27.

### Example 55: (3S,4R,5R,6S)-1-(6-{[2-(1-cyclohexen-1-yl)benzyl]oxy}hexyl)-3,4,5,6-azepanetetrol

LCMS: retention time 0.75 min, (Method A); MS (ESI, Pos.): m/z 434.2 (M+H)+;

¹H NMR (DMSO-D₆) δ: 7.39-7.32, 7.26-7.18, 7.13-7.05, 5.55-5.49, 4.67, 4.49, 4.37, 3.42-3.36, 3.22-3.13, 2.68-2.59, 2.44-2.34, 2.20-2.07, 1.75-1.57, 1.56-1.46, 1.41-1.18.

### Example 56: (3S,4R,5R,6S)-1-{6-[(2-cyclohexylbenzyl)oxy]hexyl}-3,4,5,6-azepanetetrol

LCMS: retention time 0.758 min, (Method A); MS (ESI, Pos.): m/z 436.2 (M+H)+;

¹H NMR (DMSO-D₆) δ: 7.30-7.22, 7.16-7.09, 4.67, 4.48, 4.45, 3.42, 3.22-3.13, 2.83-2.71, 2.63, 2.43-2.34, 1.81-1.68, 1.58-1.47, 1.47-1.16.

### Example 57: (3S,4R,5R,6S)-1-{6-[(4-cyclohexylbenzyl)oxy]hexyl}-3,4,5,6-azepanetetrol

LCMS: retention time 2.08 min, (Method B); MS (ESI, Pos.): m/z 436.4 (M+H)+;

¹H NMR (CDCl₃) δ: 7.26, 7.18, 4.45, 3.80-3.72, 3.64-3.57, 3.45, 2.98-2.90, 2.72-2.56, 2.52-2.45, 1.76-1.72, 1.63-1.56, 1.56-1.42, 1.42-1.28.

### Example 58: (3S,4R,5R,6S)-1-(6-{[3-(3,5-difluorophenyl)-1,2-oxazol-5-yl]methoxy}hexyl)-3,4,5,6-azepanetetrol

LCMS: retention time 1.69 min, (Method B); MS (ESI, Pos.): m/z 457.4 (M+H)+;

¹H NMR (DMSO-D₆) δ: 7.68-7.59, 7.43, 7.17, 4.68, 4.64, 4.49, 3.50, 3.39-3.32, 3.21-3.14, 2.69-2.58, 2.44-2.31, 1.59-1.48, 1.42-1.34, 1.33-1.22.

### Example 59: (3S,4R,5R,6S)-1-(6-{[5-(3,5-difluorophenyl)-1,2-oxazol-3-yl]methoxy}hexyl)-3,4,5,6-azepanetetrol

LCMS: retention time 0.783 min, (Method A); MS (ESI, Pos.): m/z 457.3 (M+H)+;

¹H NMR (CD₃OD) δ: 7.53-7.44, 7.15-7.05, 6.96, 4.60, 3.59-3.53, 3.41-3.35, 2.79, 2.56-2.47, 1.68-1.58, 1.56-1.44, 1.44-1.31.

### Example 60: (3S,4R,5R,6S)-1-{6-[(4-ethylbenzyl)oxy]hexyl}-3,4,5,6-azepanetetrol

LCMS: retention time 0.708 min, (Method A); MS (ESI, Pos.): m/z 382.1 (M+H)+;

¹H NMR (CD₃OD) δ: 7.25, 7.19, 4.45, 3.61-3.52, 3.47, 3.43-3.35, 2.80, 2.63, 2.58-2.45, 1.65-1.28, 1.22.

### Reference Example 2: (3S,4R,5R,6S)-1-{6-[(4-bromobenzyl)oxy]hexyl}-3,4,5,6-azepanetetrol

Titled compound was synthesized according to procedures analogous to Reference Example 1 and Example 1 using (4-bromolphenyl) methanol (CAS No. 873-75-6) instead of (4-methylphenyl) methanol.

LCMS: retention time 0.702 min, (Method A); MS (ESI, Pos.): m/z 431.8, 433.8 [M+H]+.

### Example 61: (3S,4R,5R,6S)-1-(6-{[4-(3,6-dihydro-2H-pyran-4-yl)benzyl]oxy}hexyl)-3,4,5,6-azepanetetrol

To a solution of the compound prepared in Reference Example 2 (150 mg) in 1,4-dioxane (2 mL) and water (0.5 mL) was added K₂CO₃ (144 mg), 2-(3,6-dihydro-2H-pyran-4-yl)-4,4,5,5-tetramethyl-1,3,2-dioxaborolane (CAS No. 287944-16-5, 109 mg) and Pd(dppf)Cl₂ (12.7 mg) at 20~25° C. The reaction mixture was stirred at 100° C. for 16 h to give a brown solution. The reaction mixture was concentrated to dryness. The residue was purified by reversed phase column (30~40% MeCN in water) to afford the titled compound (35 mg).

LCMS: retention time 0.69 min, (Method A); MS (ESI, Pos.): m/z 436.1 (M+H)+;

¹H NMR (CD₃OD) δ: 7.42, 7.31, 6.25-6.14, 4.48, 4.30, 3.92, 3.59, 3.49, 3.44-3.37, 2.83, 2.65-2.46, 1.66-1.57, 1.66-1.57, 1.45-1.36, 1.36-1.27.

### Example 62 to 67

Example 62 to 67 were synthesized according to procedures analogous to Example 62 using the compound prepared in Reference Example 2 and the corresponding boronic acid or boronic ester instead of 2-(3,6-dihydro-2H-pyran-4-yl)-4,4,5,5-tetramethyl-1,3,2-dioxaborolane.

### Example 62: (3S,4R,5R,6S)-1-{6-[(4′-fluoro-4-biphenylyl)methoxy]hexyl}-3,4,5,6-azepanetetrol

LCMS: retention time 0.739 min, (Method A); MS (ESI, Pos.): m/z 448.1 (M+H)+;

¹H NMR (CDCl₃) δ: 7.58-7.48, 7.40, 7.12, 4.53, 3.65, 3.59-3.52, 3.49, 2.88, 2.62-2.47, 1.68-1.60, 1.51-1.24.

### Example 63: 4′-[({6-[(3S,4R,5R,6S)-3,4,5,6-tetrahydroxy-1-azepanyl]hexyl}oxy)methyl]-3-biphenylcarbonitrile

LCMS: retention time 0.716 min, (Method A); MS (ESI, Pos.): m/z 455.1 (M+H)+;

¹H NMR (CD₃OD) δ: 7.99, 7.95, 7.75-7.58, 7.46, 4.55, 3.63-3.56, 3.52, 3.45-3.31, 2.90-3.75, 2.62-2.45, 1.71-1.59, 1.56-1.45, 1.45-1.39, 1.39-1.28.

### Example 64: (3S,4R,5R,6S)-1-{6-[(4′-fluoro-3′-methoxy-4-biphenylyl)methoxy]hexyl}-3,4,5,6-azepanetetrol

LCMS: retention time 0.729 min, (Method A); MS (ESI, Pos.): m/z 478.1 (M+H)+;

¹H NMR (CD₃OD) δ: 7.59, 7.41, 7.33-7.26, 7.18-7.12, 4.53, 3.94, 3.61, 3.52, 3.46-3.37, 2.85, 2.68-2.49, 1.69-1.58, 1.58-1.46, 1.46-1.28.

### Example 65: 4′-[({6-[(3S,4R,5R,6S)-3,4,5,6-tetrahydroxy-1-azepanyl]hexyl}oxy)methyl]-4-biphenylcarbonitrile

LCMS: retention time 0.721 min, (Method A); MS (ESI, Pos.): m/z 455.1 (M+H)+;

¹H NMR (CD₃OD) δ: 7.86-7.77, 7.68, 7.48, 4.56, 3.67, 3.53, 3.45, 2.89, 2.64, 1.69-1.60, 1.55, 1.49-1.39, 1.39-1.30.

### Example 66: 4′-[({6-[(3S,4R,5R,6S)-3,4,5,6-tetrahydroxy-1-azepanyl]hexyl}oxy)methyl]-2-biphenylcarbonitrile

LCMS: retention time 0.715 min, (Method A); MS (ESI, Pos.): m/z 455.1 (M+H)+;

¹H NMR (CD₃OD) δ: 7.82, 7.78-7.70, 7.65-7.44, 4.58, 3.62-3.51, 3.44-3.35, 2.82, 2.61-2.45, 1.69-1.58, 1.56-1.47, 1.47-1.39, 1.39-1.25.

### Example 67: (3S,4R,5R,6S)-1-{6-[(3′-fluoro-5′-methoxy-4-biphenylyl)methoxy]hexyl}-3,4,5,6-azepanetetrol

LCMS: retention time 0.75 min, (Method A); MS (ESI, Pos.): m/z 478.1 (M+H)+;

¹H NMR (CD₃OD) δ: 7.60, 7.42, 7.03-6.90, 6.69, 4.54, 3.85, 3.71-3.57, 3.52, 3.43, 2.96-2.79, 2.76-2.50, 1.68-1.59, 1.59-1.49, 1.47-1.39, 1.38-1.28.

### Example 68: (3S,4R,5R,6S)-1-(6-{[4-(tetrahydro-2H-pyran-4-yl)benzyl]oxy}hexyl)-3,4,5,6-azepanetetrol

To a solution of the compound prepared in Example 61 (20 mg) in methanol (1 mL) was added Pd/C (5 mg, wet, 10%) and purged with H₂ (15 psi). The reaction was stirred at 20-25° C. for 15 h to give a black suspension. The reaction mixture was filtered and the filtrate was concentrated to dryness. The mixture was purified by prep-HPLC (neutral) and was concentrated to afford the title compound (18.2 mg).

LCMS: retention time 0.689 min, (Method A); MS (ESI, Pos.): m/z 438.1 (M+H)+;

¹H NMR (CD₃OD) δ: 7.28, 7.24, 4.46, 4.08-3.98, 3.66, 3.61-3.52, 3.51-3.40, 2.99-2.84, 2.84-2.74, 2.74-2.48, 1.85-1.71, 1.66-1.47, 1.46-1.26.

### Reference Example 3: methyl 4-phenoxy-2-pyrimidinecarboxylate

To a solution of 2-chloro-4-phenoxypyrimidine (CAS No. 18214-00-1, 2.0 g) in methanol (115 mL) and toluene (23 mL) was added TEA (4.04 mL) and Pd(dppf)Cl₂ (708 mg). The mixture was heated to 150° C. under CO (3 MPa) for 15 h. The reaction mixture was filtered, then the filtrate was concentrated in vacuum to give a residue. The residue was diluted with H₂O and extracted with EtOAc. The combined organic layers were dried over Na₂SO₄ and concentrated in vacuum to dryness. The residue was purified by flash column with silica gel (PE to 30% EtOAc in PE) to give the titled compound (1.3 g).

LCMS: retention time 0.689 min, (Method A); MS (ESI, Pos.): m/z 230.9 [M+H]⁺

### Reference Example 4: (4-phenoxy-2-pyrimidinyl)methanol

To a mixture of the compound prepared in Reference Example 3 (500 mg) in methanol (6 mL) was added NaBH₄ (98.6 mg) at 0° C. The resulting mixture was stirred at 20~25° C. for 2 h. The reaction mixture was concentrated in vacuum. The residue was diluted with EtOAc, washed with H₂O and brine, then extracted with EtOAc. The combined organic layers were dried over Na₂SO₄ and concentrated in vacuum to give the title compound (470 mg, crude). The crude product was used for next step directly.

### Reference Example 5: 2-(bromomethyl)-4-phenoxypyrimidine

To a solution of the compound prepared in Reference Example 4 (150 mg) and PPh₃ (292 mg) in DCM (2 mL) was added CBr₄ (492 mg) in DCM (1 mL) at 0~5° C. The mixture was refluxed for 6 h. The reaction mixture was diluted with DCM, washed with H₂O, and extracted with DCM. The combined organic layers were washed with brine, dried over Na₂SO₄ and concentrated under reduced pressure. The residue was purified by prep-TLC (PE/EtOAc=3/1) to give the title compound (100 mg).

¹H NMR (CDCl₃) δ: 8.61-8.53, 7.50-7.42, 7.34-7.28, 7.21-7.15, 6.69, 4.45.

### Reference Example 6: 6-[(4-phenoxy-2-pyrimidinyl)methoxy]-1-hexanol

To a solution of the compound prepared in Reference Example 5 (150 mg) and t-BuOK (95 mg) in THF (4.5 mL) was added hexane-1,6-diol (CAS No. 629-11-8, 0.12 mL) dropwise. The mixture was stirred at 20-25° C. for 16 h. The reaction mixture was diluted with H₂O and extracted with DCM. The combined organic layers were dried over Na₂SO₄ and concentrated in vacuum. The residue was purified by prep-TLC (PE/EtOAc=1/1) to give the title compound (30 mg).

LCMS: retention time 0.756 min, (Method A); MS (ESI, Pos.): m/z 302.9 [M+H]⁺

### Reference Example 7: 6-[(4-phenoxy-2-pyrimidinyl)methoxy]hexyl 4-methylbenzenesulfonate

To a solution of the compound prepared in Reference Example 6 (45 mg) in DCM (0.64 mL) and pyridine (0.010 mL) was added TsCl (34 mg) at 0~5° C. The mixture was stirred at 20~25° C. for 5 h. The reaction mixture was diluted with DCM, washed with H₂O, and extracted with DCM. The combined organic layers were separated, dried over Na₂SO₄ and concentrated in vacuum. The residue was purified by prep-TLC (PE/EtOAc=3/1) to give the title compound (15 mg).

LCMS: retention time 0.956 min, (Method A); MS (ESI, Pos.): m/z 457.0 [M+H]⁺.

### Example 69: (3S,4R,5R,6S)-1-{6-[(4-phenoxy-2-pyrimidinyl)methoxy]hexyl}-3,4,5,6-azepanetetrol

To a solution of the compound prepared in Reference Example 7 (15 mg) and (3S,4R,5R,6S)-azepane-3,4,5,6-tetrol hydrochloride salt (9.8 mg) in DMSO (0.18 mL) was added DIEA (0.03 mL). The resulting mixture was heated at 80-90° C. for 2 h. The mixture was purified by prep-HPLC (base) to give the title compound (6.6 mg).

LCMS: retention time 0.667 min, (Method A); MS (ESI, Pos.): m/z 448 (M+H)+;

¹H NMR (CD₃OD) δ: 8.44, 7.35-7.21, 7.02-6.88, 6.79, 5.18, 4.38, 3.71, 3.48, 3.06-2.60, 1.84-1.70, 1.63-1.29.

### Reference Example 8: 1-bromo-2-({[6-(trityloxy)hexyl]oxy}methyl)benzene

To a mixture of (2-bromophenyl)methanol (CAS No. 18982-54-2, 500 mg) in DMF (5 mL) was added NaH (160 mg, 60% in oil) at 0-5° C. The mixture was stirred at 0-5° C. for 0.5 h. Then reaction mixture was added (((6-bromohexyl)oxy)methanetriyl)tribenzene (CAS No. 113354-79-3, 2.3 g) and KI (44.3 mg) at 0-5° C. The reaction mixture was stirred at 20° C. for 1.5 h to give a brown solution. The reaction mixture was poured into water and extracted with EtOAc. The combined organic phases were washed with water, dried over Na₂SO₄, filtered and concentrated to dryness. The residue was purified by flash column with silica gel (PE to 0% EtOAc in PE) to afford the title compound (1.1 g).

TLC R_{f}=0.5 (PE/EtOAc=1/1).

### Reference Example 9: 6-[(2-bromobenzyl)oxy]-1-hexanol

To a solution of the compound prepared in Reference Example 8 (1.1 g,) in DCM (12 mL) was added formic acid (0.80 mL) at 20° C. The mixture was stirred at 20° C. for 17 h to give a colorless solution. The reaction mixture was poured into H₂O, extracted with DCM. The combined organic layers were dried over anhydrous Na₂SO₄, filtered and concentrated. The residue was purified by flash column with silica gel (PE to 25% EtOAc in PE) to afford the title compound (300 mg).

TLC R_{f}=0.3 (PE/EtOAc=3/1).

### Reference Example 10: 6-{[2-(4,4,5,5-tetramethyl-1,3,2-dioxaborolan-2-yl)benzyl]oxy}-1-hexanol

To a solution of the compound prepared in Reference Example 9 (300 mg) in 1,4-Dioxane (4 mL) was added KOAc (308 mg), 4,4,4′,4′,5,5,5′,5′-octamethyl-2,2′-bi(1,3,2-dioxaborolane) (531 mg) and Pd(dppf)Cl₂ (38.2 mg) at 20-25° C. The reaction mixture was stirred at 80° C. for 15 h under N₂. The reaction mixture was filtered. The filtrate was poured into water and extracted with EtOAc. The combined organic phases were dried over Na₂SO₄, filtered and concentrated to afford the title compound (300 mg).

LCMS: retention time 0.927 min, (Method A); MS (ESI, Pos.): m/z 357.1 [M+Na]⁺

### Reference Example 11: 6-{[2-(4,6-difluoro-2-pyridinyl)benzyl]oxy}-1-hexanol

To a solution of 2-bromo-4,6-difluoropyridine (CAS No. 41404-63-1, 100 mg) in 1,4-dioxane (5 mL) and water (1 mL) were added K₂CO₃ (214 mg), the compound prepared in Reference Example 10 (258 mg) and Pd(dppf)Cl₂ (37.7 mg) at 20-25° C. The reaction mixture was stirred at 80° C. for 4 h to give a yellow suspension. The reaction mixture was filtered. The filtrate was poured into water and extracted with EtOAc. The combined organic phases were washed with brine, dried over Na₂SO₄, filtered and concentrated. The residue was purified by flash column with silica gel (PE to 20% EtOAc in PE) to afford the title compound (190 mg).

TLC R_{f}=0.3 (PE/EtOAc=3/1).

### Reference Example 12: 6-{[2-(4,6-difluoro-2-pyridinyl)benzyl]oxy}hexanal

To a solution of the compound prepared in Reference Example 11 (190 mg) in DCM (4 mL) was added DMP (377 mg) at 20° C. The reaction mixture was stirred at 20° C. for 1 h. The reaction mixture was quenched by sat. Na₂S₂O₃ aq. and extracted with DCM. The combined organic phases were dried over Na₂SO₄, filtered and concentrated. The residue was purified by flash column with silica gel (PE to 10% EtOAc in PE) to afford the title compound (60.0 mg).

TLC R_{f}=0.50 (PE/EtOAc=3/1).

### Example 70: (3S,4R,5R,6S)-1-(6-{[2-(4,6-difluoro-2-pyridinyl)benzyl]oxy}hexyl)-3,4,5,6-azepanetetrol

To a mixture of the compound prepared in Reference Example 12 (60 mg) in methanol (3 mL) was added (3S,4R,5R,6S)-azepane-3,4,5,6-tetrol hydrochloride salt (41 mg) and NaOAc (73 mg) at 20° C. The reaction mixture was stirred at 20° C. for 0.5 h to give a yellow solution. Then NaBH₃CN (35 mg) was added in the mixture at 20° C. The reaction mixture was stirred at 20° C. for 15 h. The reaction mixture was concentrated to dryness. The residue was purified by prep-HPLC (neutral) to afford the title compound (34 mg).

LCMS: retention time 0.693 min, (Method A); MS (ESI, Pos.): m/z 489.1 (M+Na)+;

¹H NMR (DMSO-D₆) δ: 7.59-7.53, 7.52-7.47, 7.46-7.40, 7.31-7.22, 4.66, 4.57, 4.48, 3.40-3.36, 3.32-3.29, 3.19-3.15, 2.62, 2.37, 1.43-1.29, 1.21-1.13.

### Reference Example 13: 1-[(4-ethoxy-3-fluorobenzyl)oxy]-6-[(triisopropylsilyl)oxy]-2-hexanol

To a solution of (3-ethoxy-4-fluorophenyl)methanol (CAS No. 1000207-64-6, 800 mg) in t-BuOH (5 mL) was added 15% NaOHaq. (5 mL), triisopropyl(4-(oxiran-2-yl)butoxy)silane (CAS No. 2734792-44-8, 1.41 g) and TBAI (758 mg) at 20~25° C. The resulting mixture was purged with N₂ for 3 times and stirred at 90° C. for 15 h. The reaction mixture was poured into water and extracted with EtOAc. The combined organic layers were dried over Na₂SO₄ and concentrated in vacuo. The residue was purified by flash column with silica gel (PE to 15% of EtOAc in PE) to afford the title compound (660 mg).

TLC R_{f}=0.40 (PE/EtOAc=5/1).

### Reference Example 14: ({6-[(4-ethoxy-3-fluorobenzyl)oxy]-5-fluorohexyl}oxy)(triisopropyl)silane

To a solution of the compound prepared in Reference Example 13 (660 mg) in DCM (1 mL) was added DAST (0.26 mL) at 0° C. The mixture was stirred for 5 min. The mixture was quenched by sat. NaHCO₃ aq., washed with water, extracted with DCM. The combined organic layers were concentrated and purified by flash column with silica gel (PE to 5% EtOAc in PE,) to give the title compound (160 mg).

TLC R_{f}=0.60 (PE/EtOAc=10/1).

### Reference Example 15: 6-[(4-ethoxy-3-fluorobenzyl)oxy]-5-fluoro-1-hexanol

To a mixture of the compound prepared in Reference Example 14 (160 mg) in THF (2 mL) was added 1M TBAF in THF (0.54 mL) at 20° C. The reaction was stirred at 20° C. for 1 h to give a yellow solution. The reaction was concentrated to dryness. The residue was directly purified by flash column with silica gel (PE to 35% EtOAc in PE,) to afford the title compound (90 mg).

TLC R_{f}=0.20 (PE/EtOAc=2/1).

### Reference Example 16: 6-[(4-ethoxy-3-fluorobenzyl)oxy]-5-fluorohexyl 4-methylbenzenesulfonate

To a solution of the compound prepared in Reference Example 15 (85 mg) in DCM (2 mL) was added 4-methyl benzene-1-sulfonyl chloride (0.090 mL), TEA (0.12 mL) and DMAP (13 mg,) at 25° C. The reaction mixture was stirred at 25° C. for 15 h. The reaction mixture was concentrated to dryness. The residue was purified by flash column with silica gel (PE to 20% EtOAc in PE,) to afford the title compound (100 mg).

TLC R_{f}=0.60 (PE/EtOAc=3/1).

### Example 71: (3S,4R,5R,6S)-1-{(5R)-6-[(3-ethoxy-4-fluorobenzyl)oxy]-5-fluorohexyl}-3,4,5,6-azepanetetrol (Isomer 1)

### Example 72: (3S,4R,5R,6S)-1-{(5S)-6-[(3-ethoxy-4-fluorobenzyl)oxy]-5-fluorohexyl}-3,4,5,6-azepanetetrol (Isomer 2)

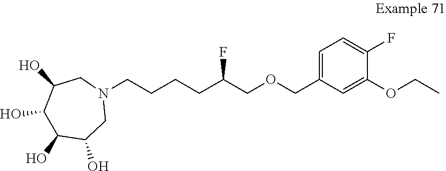 [SMILES: CCOc1cc(COC[C@H](F)CCCCN2C[C@H](O)[C@@H](O)[C@H](O)[C@@H](O)C2)ccc1F]

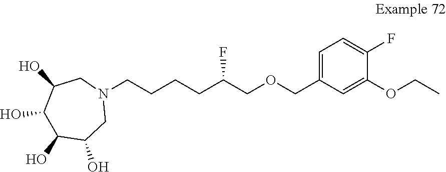 [SMILES: CCOc1cc(COC[C@@H](F)CCCCN2C[C@H](O)[C@@H](O)[C@H](O)[C@@H](O)C2)ccc1F]

To a solution of (3S,4R,5R,6S)-azepane-3,4,5,6-tetrol hydrochloride salt (68 mg) in DMSO (1.5 mL) was added DIEA (0.19 mL), KI (19 mg) and the compound prepared in Reference Example 16 (100 mg) at 20-25° C. The reaction mixture was stirred at 80° C. for 2 h. The reaction mixture was concentrated to dryness. The residue was purified by prep-HPLC (NH₃) and chiral SFC separation to obtain isomer 1 (5.0 mg) and isomer 2 (15 mg).

Example 71 (isomer 1): LCMS: retention time 0.683 min, (Method A); MS (ESI, Pos.): m/z 434.1 (M+H)+;

¹H NMR (CD₃OD) δ: 7.12-7.00, 6.92-6.84, 4.73-4.65, 4.51, 4.10, 3.67-3.50, 3.43-3.36, 2.81, 2.61-2.47, 1.74-1.35;

SFC Retention Time: 6.027 min (Method: A1).

Example 72 (isomer 2): LCMS: retention time 0.688 min, (Method A); MS (ESI, Pos.): m/z 434.1 (M+H)+;

¹H NMR (CD₃OD) δ: 7.13-7.00, 6.92-6.84, 4.75-4.65, 4.51, 4.10, 3.68-3.50, 3.44-3.35, 2.80, 2.59-2.44, 1.74-1.39;

SFC Retention Time: 6.254 min (Method: A1).

### Example 73 to 86

Example 73 to 86 were synthesized according to procedures analogous to Reference Example 13, 14, 15, 16, Example 71 and 72 using the corresponding alcohol compound instead of (3-ethoxy-4-fluorophenyl)methanol.

### Example 73: (3S,4R,5R,6S)-1-[(5R)-5-fluoro-6-{[2-(4-fluorophenyl)-1,3-thiazol-4-yl]methoxy}hexyl]-3,4,5,6-azepanetetrol (Isomer 1)

LCMS: retention time 1.612 min, (Method B); MS (ESI, Pos.): m/z 473.1 (M+H)+;

¹H NMR (CD₃OD) δ: 7.99, 7.48, 7.26-7.19, 4.75-4.57, 4.72, 3.75-3.65, 3.60-3.52, 3.41-3.37, 2.79, 2.60-2.44, 1.73-1.40;

SFC Retention Time: 4.734 min (Method: B8).

### Example 74: (3S,4R,5R,6S)-1-[(5S)-5-fluoro-6-{[2-(4-fluorophenyl)-1,3-thiazol-4-yl]methoxy}hexyl]-3,4,5,6-azepanetetrol (Isomer 2)

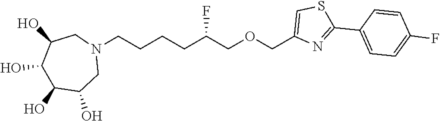 [SMILES: O[C@H]1[C@H](O)[C@@H](O)CN(CCCC[C@H](F)COCc2csc(-c3ccc(F)cc3)n2)C[C@@H]1O]

LCMS: retention time 1.62 min, (Method B); MS (ESI, Pos.): m/z 473.1 (M+H)+;

¹H NMR (CD₃OD) δ: 8.05-7.95, 7.48, 7.27-7.19, 4.75-4.57, 4.72, 3.78-3.63, 3.45, 2.90, 2.80-2.50, 1.75-1.38;

SFC Retention Time: 3.393 min (Method: B8).

### Example 75: (3S,4R,5R,6S)-1-{5-fluoro-6-[(3,4,5-trifluorobenzyl)oxy]hexyl}-3,4,5,6-azepanetetrol (Isomer 1)

LCMS: retention time 0.726 min, (Method A); MS (ESI, Pos.): m/z 426.1 (M+H)+;

¹H NMR (CD₃OD) δ: 7.13-7.10, 4.75-4.60, 4.53, 3.68-3.63, 3.61-3.53, 3.40-3.37, 2.81, 2.58-2.49, 1.74-1.30;

SFC Retention Time: 1.826 min (Method: F4).

### Example 76: (3S,4R,5R,6S)-1-{5-fluoro-6-[(3,4,5-trifluorobenzyl)oxy]hexyl}-3,4,5,6-azepanetetrol (Isomer 2)

LCMS: retention time 0.741 min, (Method A); MS (ESI, Pos.): m/z 426.1 (M+H)+;

¹H NMR (CD₃OD) δ: 7.13-7.10, 4.75-4.60, 4.53, 3.71-3.53, 3.41-3.37, 2.81, 2.60-2.49, 1.73-1.51, 1.51-1.37;

SFC Retention Time: 1.965 min (Method: F4).

### Example 77: (3S,4R,5R,6S)-1-[6-(benzyloxy)-5-fluorohexyl]-3,4,5,6-azepanetetrol (Isomer 1)

LCMS: retention time 1.384 min, (Method B); MS (ESI, Pos.): m/z 372.1 (M+H)+;

¹H NMR (CD₃OD) δ: 7.42-7.21, 4.72-4.55, 4.55, 3.68-3.50, 3.42-3.35, 2.81, 2.59-2.46, 1.73-1.28;

SFC Retention Time: 6.251 min (Method: B5).

### Example 78: (3S,4R,5R,6S)-1-[6-(benzyloxy)-5-fluorohexyl]-3,4,5,6-azepanetetrol (Isomer 2)

LCMS: retention time 1.394 min, (Method B); MS (ESI, Pos.): m/z 372.1 (M+H)+;

¹H NMR (CD₃OD) δ: 7.39-7.23, 4.72-4.55, 4.56, 3.66-3.51, 3.43-3.35, 2.80, 2.58-2.47, 1.73-1.33;

SFC Retention Time: 6.795 min (Method: B5).

### Example 79: (3S,4R,5R,6S)-1-{5-fluoro-6-[(4-fluorobenzyl)oxy]hexyl}-3,4,5,6-azepanetetrol (Isomer 1)

LCMS: retention time 1.448 min, (Method B); MS (ESI, Pos.): m/z 390.1 (M+H)+;

¹H NMR (CD₃OD) δ: 7.37, 7.07, 4.72-4.55, 3.67-3.51, 3.43-3.36, 2.80, 2.59-2.47, 1.75-1.36;

SFC Retention Time: 6.3 min (Method: B4).

### Example 80: (3S,4R,5R,6S)-1-{5-fluoro-6-[(4-fluorobenzyl)oxy]hexyl}-3,4,5,6-azepanetetrol (Isomer 2)

LCMS: retention time 0.666 min, (Method A); MS (ESI, Pos.): m/z 390 (M+H)+;

¹H NMR (CD₃OD) δ: 7.37, 7.07, 4.73-4.60, 4.53, 3.66-3.49, 3.44-3.35, 2.80, 2.59-2.46, 1.72-1.37;

SFC Retention Time: 6.799 min (Method: B4).

### Example 81: (3S,4R,5R,6S)-1-{6-[(2-chlorobenzyl)oxy]-5-fluorohexyl}-3,4,5,6-azepanetetrol (Isomer 1)

LCMS: retention time 0.696 min, (Method A); MS (ESI, Pos.): m/z 406.1 (M+H)+;

¹H NMR (CDCl₃) δ: 7.47, 7.39-7.33, 7.31-7.28, 7.25-7.20, 4.78-4.55, 4.66, 3.72-3.65, 3.65-3.61, 3.61-3.50, 2.91, 2.66-2.52, 1.55-1.41;

SFC Retention Time: 4.572 min (Method: G1).

### Example 82: (3S,4R,5R,6S)-1-{6-[(2-chlorobenzyl)oxy]-5-fluorohexyl}-3,4,5,6-azepanetetrol (Isomer 2)

LCMS: retention time 0.694 min, (Method A); MS (ESI, Pos.): m/z 406 (M+H)+;

¹H NMR (CDCl₃) δ: 7.52-7.45, 7.40-7.32, 7.31-7.28, 7.25-7.18, 4.77-4.56, 4.67, 3.73-3.58, 3.58-3.50, 2.91, 2.64-2.49, 1.58-1.35;

SFC Retention Time: 4.809 min (Method: G1).

### Example 83: (3S,4R,5R,6S)-1-(5-fluoro-6-{[2-(1-propyn-1-yl)benzyl]oxy}hexyl)-3,4,5,6-azepanetetrol (Isomer 1)

LCMS: retention time 0.687 min, (Method A); MS (ESI, Pos.): m/z 410.1 (M+H)+;

¹H NMR (CDCl₃) δ: 7.47-7.35, 7.32-7.27, 7.24-7.17, 4.82-4.55, 4.72, 3.72-3.48, 2.87, 2.64-2.49, 2.09, 1.73-1.65, 1.54-1.39;

SFC Retention Time: 4.628 min (Method: G1).

### Example 84: (3S,4R,5R,6S)-1-(5-fluoro-6-{[2-(1-propyn-1-yl)benzyl]oxy}hexyl)-3,4,5,6-azepanetetrol (Isomer 2)

LCMS: retention time 0.693 min, (Method A); MS (ESI, Pos.): m/z 410.1 (M+H)+;

¹H NMR (CDCl₃) δ: 7.46-7.36, 7.32-7.26, 7.23-7.17, 4.77-4.56, 4.74, 3.69-3.52, 2.87, 2.62-2.50, 2.08, 1.75-1.65, 1.50-1.30;

SFC Retention Time: 4.865 min (Method: G1).

### Example 85: (3S,4R,5R,6S)-1-{6-[(2-chloro-4-methylbenzyl)oxy]-5-fluorohexyl}-3,4,5,6-azepanetetrol (Isomer 1)

LCMS: retention time 0.707 min, (Method A); MS (ESI, Pos.): m/z 420.1 (M+H)+;

¹H NMR (CD₃OD) δ: 7.36, 7.22, 7.12, 4.73-4.50, 4.59, 3.71-3.52, 3.44-3.34, 2.81, 2.62-2.47, 2.33, 1.73-1.27;

SFC Retention Time: 1.855 min (Method: G2).

### Example 86: (3S,4R,5R,6S)-1-{6-[(2-chloro-4-methylbenzyl)oxy]-5-fluorohexyl}-3,4,5,6-azepanetetrol (Isomer 2)

LCMS: retention time 0.703 min, (Method A); MS (ESI, Pos.): m/z 420.1 (M+H)+;

¹H NMR (CD₃OD) δ: 7.36, 7.22, 7.13, 4.73-4.50, 4.58, 3.69-3.54, 3.42-3.36, 2.81, 2.61-2.45, 2.33, 1.74-1.28;

SFC Retention Time: 2.103 min (Method: G2).

### Example 87 to 98

Example 87 to 98 were synthesized according to procedures analogous to Reference Example 13, 14, 15, 16 and Example 69 using the corresponding alcohol compound instead of (3-ethoxy-4-fluorophenyl)methanol.

### Example 87: (3S,4R,5R,6S)-1-(6-{[2-(3,5-difluorophenyl)-1,3-thiazol-4-yl]methoxy}-5-fluorohexyl)-3,4,5,6-azepanetetrol (Mixture of Two Isomer)

LCMS: retention time 0.711 min, (Method A); MS (ESI, Pos.): m/z 491.1 (M+H)+;

¹H NMR (DMSO-D₆) δ: 7.75, 7.68-7.60, 7.45-7.36, 4.77-4.67, 4.66, 4.65-4.58, 4.49, 3.76-3.58, 3.37, 3.19, 2.64, 2.44-2.31, 1.65-1.28;

SFC Retention Time: 3.525/3.851 min (Method: B1).

### Example 88: (3S,4R,5R,6S)-1-{6-[(3′,5′-difluoro-2-biphenylyl)methoxy]-5-fluorohexyl}-3,4,5,6-azepanetetrol (Mixture of Two Isomer)

LCMS: retention time 1.802 min, (Method B); MS (ESI, Pos.): m/z 484.4 (M+H)+;

¹H NMR (CD₃OD) δ: 7.66-7.26, 7.19-6.85, 4.70-4.65, 4.57-4.38, 3.67-3.50, 3.43, 2.87, 2.80-2.43, 1.76-1.35;

SFC Retention Time: 2.082/2.139 min (Method: E1).

### Example 89: (3S,4R,5R,6S)-1-(6-{[2-(3-chloro-5-fluorophenyl)-1,3-thiazol-4-yl]methoxy}-5-fluorohexyl)-3,4,5,6-azepanetetrol (Mixture of Two Isomer)

LCMS: retention time 1.793 min, (Method B); MS (ESI, Pos.): m/z 507.4 (M+H)+;

¹H NMR (CD₃OD) δ: 7.82, 7.72-7.64, 7.57, 7.36-7.29, 4.72, 4.61-4.53, 3.79-3.65, 3.60-3.53, 3.42-3.35, 2.86-2.74, 2.57-2.47, 1.75-1.33.

### Example 90: (3S,4R,5R,6S)-1-(6-{[2-(3-chloro-5-methoxyphenyl)-1,3-thiazol-4-yl]methoxy}-5-fluorohexyl)-3,4,5,6-azepanetetrol (Mixture of Two Isomer)

LCMS: retention time 1.779 min, (Method B); MS (ESI, Pos.): m/z 519.4 (M+H)+;

¹H NMR (CD₃OD) δ: 7.57-7.49, 7.48-7.42, 7.07, 4.72, 4.59, 3.88, 3.78-3.65, 3.57, 3.43-3.34, 2.80, 2.59-2.44, 1.75-1.32.

### Example 91: (3S,4R,5R,6S)-1-{5-fluoro-6-[(3′-fluoro-2-biphenylyl)methoxy]hexyl}-3,4,5,6-azepanetetrol Hydrochloride (1:1) (Mixture of Two Isomer)

LCMS: retention time 0.735 min, (Method A); MS (ESI, Pos.): m/z 466.2 (M+H)+;

¹H NMR (CD₃OD) δ: 7.56-7.49, 7.47-7.37, 7.33-7.27, 7.23-7.07, 4.71-4.50, 4.48-4.34, 4.19-4.03, 3.84, 3.65-3.55, 3.54-3.49, 3.41, 3.35, 3.29-3.12, 1.94-1.39;

SFC Retention Time: 2.298/2.342 min (Method: E1).

### Example 92: (3S,4R,5R,6S)-1-{6-[(3′,4′-difluoro-2-biphenylyl)methoxy]-5-fluorohexyl}-3,4,5,6-azepanetetrol (Mixture of Two Isomer)

LCMS: retention time 0.743 min, (Method A); MS (ESI, Pos.): m/z 484.1 (M+H)+;

¹H NMR (DMSO-D₆) δ: 7.55-7.45, 7.45-7.37, 7.32-7.27, 7.24, 4.78-4.43, 4.42-4.32, 3.57-3.42, 3.37, 3.18, 2.64, 2.44-2.29, 1.62-1.24;

SFC Retention Time: 2.275/2.393 min (Method: E1).

### Example 93: (3S,4R,5R,6S)-1-{5-fluoro-6-[(4-propylbenzyl)oxy]hexyl}-3,4,5,6-azepanetetrol (Mixture of Two Isomer)

LCMS: retention time 1.789 min, (Method B); MS (ESI, Pos.): m/z 414.2 (M+H)+;

¹H NMR (CD₃OD) δ: 7.26, 7.17, 4.71-4.50, 4.52, 3.62-3.53, 3.39, 2.82, 2.61-2.50, 1.68-1.37, 0.93.

### Example 94: (3S,4R,5R,6S)-1-(5-fluoro-6-{[2-(3,4,5-trifluorophenyl)-1,3-thiazol-4-yl]methoxy}hexyl)-3,4,5,6-azepanetetrol (Mixture of Two Isomer)

LCMS: retention time 0.718 min, (Method A); MS (ESI, Pos.): m/z 531.1 (M+Na) (M+H)+;

¹H NMR (CD₃OD) δ: 7.80-7.71, 7.56, 4.71, 4.60-4.56, 3.75-3.71, 3.69-3.66, 3.60-3.52, 3.41-3.35, 2.79, 2.57-2.49, 1.74-1.58, 1.55-1.40;

SFC Retention Time: 3.254/3.307 min (Method: J1).

### Example 95: (3S,4R,5R,6S)-1-(5-fluoro-6-{[2-(2,3,4-trifluorophenyl)-1,3-thiazol-4-yl]methoxy}hexyl)-3,4,5,6-azepanetetrol (Mixture of Two Isomer)

LCMS: retention time 0.734 min, (Method A); MS (ESI, Pos.): m/z 531.1 (M+Na) (M+H)+;

¹H NMR (CD₃OD) δ: 8.09-8.00, 7.66, 7.35-7.22, 4.75, 4.63-4.56, 3.79-3.65, 3.65-3.55, 3.46-3.37, 2.84, 2.67-2.48, 1.76-1.35;

SFC Retention Time: 2.657/2.747 min (Method: E1).

### Example 96: (3S,4R,5R,6S)-1-(6-{[5-(3,5-difluorophenyl)-1,2-oxazol-3-yl]methoxy}-5-fluorohexyl)-3,4,5,6-azepanetetrol (Mixture of Two Isomer)

LCMS: retention time 0.799 min, (Method A); MS (ESI, Pos.): m/z 475.2 (M+H)+;

¹H NMR (CDCl₃) δ: 7.33-7.27, 6.95-6.81, 6.65, 4.80-4.51, 4.70, 3.76, 3.69-3.53, 2.94, 2.81-2.47, 1.83-1.32.

### Example 97: (3S,4R,5R,6S)-1-{5-fluoro-6-[(5-methyl-2-propyl-1,3-thiazol-4-yl)methoxy]hexyl}-3,4,5,6-azepanetetrol (Mixture of Two Isomer)

LCMS: retention time 1.531 min, (Method B); MS (ESI, Pos.): m/z 435.2 (M+H)+;

¹H NMR (DMSO-D₆) δ: 4.82-4.50, 4.50-4.34, 3.60-3.37, 3.21-3.14, 2.82, 2.69-2.59, 2.44-2.31, 1.67, 1.59-1.19, 0.92;

SFC Retention Time: 3.529/3.618 min (Method: D1).

### Example 98: (3S,4R,5R,6S)-1-{5-fluoro-6-[(4-isopropylbenzyl)oxy]hexyl}-3,4,5,6-azepanetetrol (Mixture of Two Isomer)

LCMS: retention time 1.811 min, (Method B); MS (ESI, Pos.): m/z 414.2 (M+H)+;

¹H NMR (DMSO-D₆) δ: 7.27-7.19, 4.71, 4.62-4.36, 3.63-3.41, 3.19, 2.94-2.80, 2.69-2.60, 2.52-2.51, 2.46-2.30, 1.66-1.28, 1.19;

SFC Retention Time: 4.438/4.795 min (Method: B2).

### Reference Example 17: 2-fluoro-6-[(triisopropylsilyl)oxy]hexanal

A solution of NFSI (90 g) and DL-proline (33 g) in THF (560 mL) and IPA (70 mL) was stirred at 20° C. for 15 min. Then a solution of 6-((triisopropylsilyl)oxy)hexanal (CAS No. 245124-09-8, 78 g) in THF (100 mL) was added. The mixture was stirred at 20° C. for 16 h. To the reaction mixture was added MTBE. The mixture was stirred at −78° C. for 30 min, then filtered. The filtrate was washed with sat. NaHCO₃ aq. and brine, dried over Na₂SO₄ and concentrated to give the title compound (78 g)), which was used in the next step without any further purification.

### Reference Example 18: 2-fluoro-6-[(triisopropylsilyl)oxy]-1-hexanol

To a solution of the compound prepared in Reference Example 17 (39 g) in DCM (400 mL) and EtOH (400 mL) was added NaBH₄ (28 g) at 0° C. The mixture was stirred at 0° C. for 0.5 h. The reaction mixture was quenched by sat. NH₄Cl aq. (500 mL) at 0° C. The mixture was stirred vigorously for 0.5 h. The mixture was extracted with DCM, washed with sat. NaHCO₃ aq.. The combined organic layers were concentrated to give the crude product. Two batches of crude product were combined and purified by column chromatography with silica gel (EtOAc in PE=0~4%) to give the title compound (30 g).

¹H NMR (CD₃OD) δ: 4.58-4.38, 3.77-3.65, 1.84-1.62, 1.56-1.33, 109-1.04.

### Reference Example 19: methyl 5-(3,5-difluorophenyl)-2-methyl-1,3-oxazole-4-carboxylate

Titled compound was synthesized according to procedures analogous to Reference Example 11 using methyl 5-bromo-2-methyloxazole-4-carboxylate (CAS No. 1935479-67-6) and (3,5-difluorophenyl)boronic acid (CAS No. 156545-07-2) and was used in the next step without any further purification.

### Reference Example 20: [5-(3,5-difluorophenyl)-2-methyl-1,3-oxazol-4-yl]methanol

To a solution of the compound prepared in Reference Example 19 (5.00 g) in THF (200 mL) was added 3.5M Red-A1 in toluene (7 mL) at 0° C. The mixture was stirred at 0° C. for 15 min. 1M HClaq. was added to quench the reaction, the mixture was stirred for 10 min. The mixture was washed with water and extracted with EtOAc. The water phase was washed with sat NaCl aq. and extracted with EtOAc. The combined organic layers were dried over Na₂SO₄. The crude product (5.00 g×6 batches) was purified by column chromatography with silica gel (PE to 28% EtOAc in PE) to give the title compound (14.5 g).

TLC R_{f}=0.40 (PE/EtOAc=1/1)

### Reference Example 21: 4-(bromomethyl)-5-(3,5-difluorophenyl)-2-methyl-1,3-oxazole

A mixture of the compound prepared in Reference Example 20 (12 g) in DCM (150 mL) was heated to dissolve. After the mixture was cooled, DIEA (19 mL) and LiBr (14 g) were added to the mixture at 0° C. The mixture was stirred at 0° C. for 10 min. Then Ms₂O (12 g) was added and the mixture was stirred at 30° C. for 1 h. The mixture was washed with water and extracted with DCM. The combined organic layers were dried over Na₂SO₄ and concentrated under reduced pressure. The residue was purified by column with silica gel (PE to 10% EtOAc in PE,) to give the title compound (12 g).

TLC R_{f}=0.70 (PE/EtOAc=3/1).

### Reference Example 22: 5-(3,5-difluorophenyl)-4-[({2-fluoro-6-[(triisopropylsilyl)oxy]hexyl}oxy)methyl]-2-methyl-1,3-oxazole

To a solution of the compound prepared in Reference Example 18 (17 g) in THF (300 mL) was added NaH (4.7 g, 60% in oil) at 0° C. The mixture was stirred at 0° C. for 30 min. The compound prepared in Reference Example 21 (17 g) was added and the mixture was stirred at 0° C. for 16 h. The mixture was washed with water and extracted with EtOAc. The combined organic layers were dried over Na₂SO₄ and concentrated under reduced pressure. The residue was purified by silica column (PE to 5% EtOAc in PE,) to give the title compound (22 g).

TLC R_{f}=0.8 (PE/EtOAc=3/1)

### Example 99: (3S,4R,5R,6S)-1-[(5R)-6-{[5-(3,5-difluorophenyl)-2-methyl-1,3-oxazol-4-yl]methoxy}-5-fluorohexyl]-3,4,5,6-azepanetetrol (Isomer 1)

### Example 100: (3S,4R,5R,6S)-1-[(5S)-6-{[5-(3,5-difluorophenyl)-2-methyl-1,3-oxazol-4-yl]methoxy}-5-fluorohexyl]-3,4,5,6-azepanetetrol (Isomer 2)

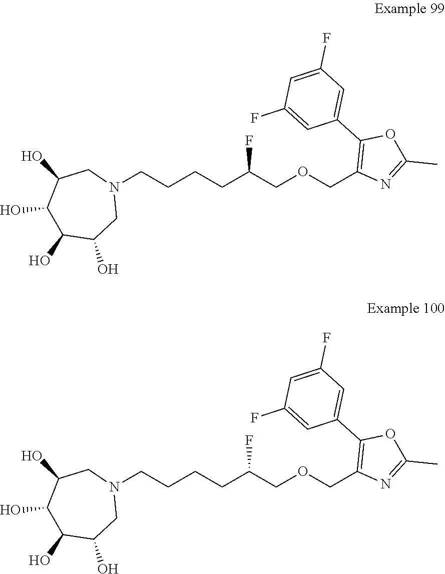 [SMILES: Cc1nc(COC[C@H](F)CCCCN2C[C@H](O)[C@@H](O)[C@H](O)[C@@H](O)C2)c(-c2cc(F)cc(F)c2)o1 ; Cc1nc(COC[C@@H](F)CCCCN2C[C@H](O)[C@@H](O)[C@H](O)[C@@H](O)C2)c(-c2cc(F)cc(F)c2)o1]

Titled compounds were synthesized according to procedures analogous to Reference Example 15, 16, Example 71 and 72 using the compound prepared in Reference Example 22 instead of the compound prepared in Reference Example 14.

Example 99 (isomer 1): LCMS: retention time 0.681 min, (Method A); MS (ESI, Pos.): m/z 489.1 (M+H)+;

¹H NMR (CD₃OD) δ: 7.40-7.27, 7.05-6.95, 4.73-4.55, 4.60, 3.76-3.53, 3.43-3.35, 2.79, 2.57-2.45, 1.73-1.33;

SFC Retention Time: 3.312 min (Method: A2).

Example 100 (isomer 2): LCMS: retention time 0.688 min, (Method A); MS (ESI, Pos.): m/z 489.1 (M+H)+;

¹H NMR (CD₃OD) δ: 7.40-7.27, 7.05-6.95, 4.73-4.55, 4.60, 3.76-3.53, 3.43-3.35, 2.79, 2.57-2.45, 1.73-1.33;

SFC Retention Time: 4.893 min (Method: A2).

### Reference Example 23: 1-[(4-ethylbenzyl)oxy]-6-[(triisopropylsilyl)oxy]-2-hexanol

To a solution of (4-ethylphenyl)methanol (CAS No. 768-59-2, 66 g) and triisopropyl(4-(oxiran-2-yl)butoxy)silane (158 g) in t-BuOH (150 mL) was added TBAI (31 g) and 15% NaOHaq. (150 mL) at 20° C. The reaction was stirred at 80° C. for 16 h. The mixture was extracted with EtOAc, washed with brine. The organic phase was concentrated and purified by flash column with silica gel (EtOAc in PE=0~3%) to give the title compound (34 g).

¹H NMR (CDCl₃) δ: 7.26, 7.20-7.16, 4.52, 3.86-3.75, 3.68, 3.52-3.47, 3.31, 2.65, 1.49-1.38, 1.28-1.18, 1.06-1.04.

### Reference Example 24: 6-[(4-ethylbenzyl)oxy]-1,5-hexanediol

To a solution of the compound prepared in Reference Example 23 (60 g) in THF (400 mL) was added TBAF (152 mL) at 20° C. The reaction was stirred at 20° C. for 15 h. The reaction mixture was poured into water and extracted with EtOAc. The combined organic layers were washed with brine, dried over Na₂SO₄, filtered and concentrated. The residue was purified by flash column with silica gel (EtOAc in PE=55~65%) to give the title compound (30 g).

¹H NMR (CDCl₃) δ: 7.26-7.22, 7.20-7.16, 4.51, 3.86-3.76, 3.61, 3.48, 3.31, 2.64, 1.63-1.51, 1.48-1.37, 1.26-1.20.

### Reference Example 25: 6-[(4-ethylbenzyl)oxy]-5-hydroxyhexyl 4-methylbenzenesulfonate

To a solution of the compound prepared in Reference Example 24 in DCM (400 mL) was added TsCl (33 g) and TEA (72 mL) at 0° C. The mixture was stirred at 20° C. for 16 h. The mixture was washed with water and extracted by DCM. The combined organic layers were dried over Na₂SO₄ and concentrated under reduced pressure. The residue was purified by silica column chromatography (PE to 35% EtOAc in PE) to give the title compound (33 g).

¹H NMR (CDCl₃) δ: 7.77, 7.35-7.29, 7.25-7.21, 7.20-7.15, 4.49, 4.01, 3.76-3.67, 3.43, 3.27, 2.64, 2.44-2.42, 1.71-1.59, 1.42-1.31, 1.23.

### Reference Example 26: 6-[(4-ethylbenzyl)oxy]-5-fluorohexyl 4-methylbenzenesulfonate

To a solution of the compound prepared in Reference Example 25 (39 g) in THF (400 mL) was added PBSF (33 mL), hydrogen fluoride in triethylamine (31 mL) and TEA (80 mL) at 0° C. The reaction was stirred for 16 h. The reaction mixture was poured into water and extracted with EtOAc. The combined organic layers were dried over Na₂SO₄ and concentrated. The residue was purified by flash column with silica gel (PE to 4% EtOAc in PE) to afford the title compound (12 g).

¹H NMR (CDCl₃) δ: 7.78, 7.34, 7.26 (s, 2H), 7.21-7.17, 4.64, 4.55-4.48, 4.03, 3.57-3.47, 2.65, 2.44, 1.73-1.63, 1.56-1.33, 1.23.

### Example 101: (3S,4R,5R,6S)-1-{(5S)-6-[(4-ethylbenzyl)oxy]-5-fluorohexyl}-3,4,5,6-azepanetetrol (Isomer 1)

### Example 102: (3S,4R,5R,6S)-1-{(5R)-6-[(4-ethylbenzyl)oxy]-5-fluorohexyl}-3,4,5,6-azepanetetrol (Isomer 2)

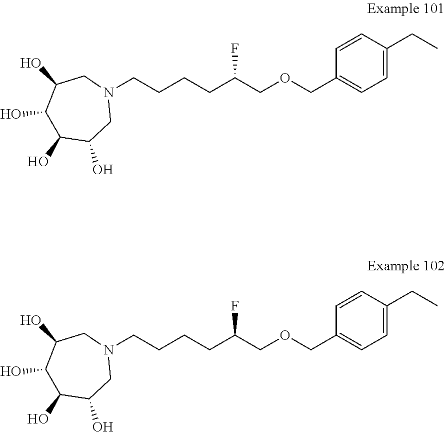 [SMILES: CCc1ccc(COC[C@@H](F)CCCCN2C[C@H](O)[C@@H](O)[C@H](O)[C@@H](O)C2)cc1 ; CCc1ccc(COC[C@H](F)CCCCN2C[C@H](O)[C@@H](O)[C@H](O)[C@@H](O)C2)cc1]

To a solution of the compound prepared in Reference Example 26 (16 g) in DMF (150 mL) was added (3S,4R,5R,6S)-azepane-3,4,5,6-tetrol hydrochloride salt (9.5 g), KI (12 g) and DIEA (33 mL) at 25° C. The reaction mixture was stirred at 80° C. for 15 h. The mixture was treated with NaHCO₃ and extracted by EtOAc. The combined organic layers were washed with water. The combined organic layers were dried over Na₂SO₄ and concentrated under reduced pressure to obtain crude product of titled compounds (14 g). The crude products (36 g) from two batches was purified by SFC to give the title compound (isomer 1, 14 g) and the title compound (isomer 2, 14 g).

Example 101 (isomer 1): LCMS: retention time 0.697 min, (Method A); MS (ESI, Pos.): m/z 400.1 (M+H)+;

¹H NMR (CD₃OD) δ: 7.26, 7.20, 4.70-4.54, 4.52, 3.61-3.51, 3.43-3.36, 2.80, 2.67-2.59, 2.59-2.45, 1.73-1.31, 1.22;

SFC Retention Time: 2.827 min (Method: B6).

Example 102 (isomer 2): LCMS: retention time 0.691 min, (Method A); MS (ESI, Pos.): m/z 400.1 (M+H)+;

¹H NMR (CD₃OD) δ: 7.26, 7.20, 4.73-4.54, 4.52, 3.67-3.51, 3.48-3.37, 2.89-2.68, 2.67-2.48, 1.76-1.56, 1.56-1.32, 1.22;

SFC Retention Time: 3.852 min (Method: B6).

### Example 103 to 134

Example 103 to 134 were synthesized according to procedures analogous to Reference Example 23, 24, 25, 26, Example 71 and 72 using the corresponding alcohol compound instead of (4-ethylphenyl)methanol.

### Example 103: (3S,4R,5R,6S)-1-[(5R)-5-fluoro-6-{[2-(3-methoxyphenyl)-1,3-thiazol-4-yl]methoxy}hexyl]-3,4,5,6-azepanetetrol (Isomer 1)

LCMS: retention time 0.715 min, (Method A); MS (ESI, Pos.): m/z 485.1 (M+H)+;

¹H NMR (CD₃OD) δ: 7.56-7.44, 7.38, 7.04, 4.71, 4.75-4.56, 3.87, 3.77-3.64, 3.60-3.51, 3.41-3.35, 2.79, 2.58-2.44, 1.74-1.34;

SFC Retention Time: 5.341 min (Method: B7).

### Example 104: (3S,4R,5R,6S)-1-[(5S)-5-fluoro-6-{[2-(3-methoxyphenyl)-1,3-thiazol-4-yl]methoxy}hexyl]-3,4,5,6-azepanetetrol (Isomer 2)

LCMS: retention time 0.711 min, (Method A); MS (ESI, Pos.): m/z 485.1 (M+H)+;

¹H NMR (CD₃OD) δ: 7.54-7.46, 7.38, 7.06-7.01, 4.71, 4.75-4.59, 3.87, 3.76-3.66, 3.60-3.52, 3.42-3.35, 2.79, 2.60-2.42, 1.75-1.60, 1.57-1.34;

SFC Retention Time: 6.714 min (Method: B7).

### Example 105: (3S,4R,5R,6S)-1-(6-{[2-(3,4-difluorophenyl)-1,3-thiazol-4-yl]methoxy}-5-fluorohexyl)-3,4,5,6-azepanetetrol (Isomer 1)

LCMS: retention time 1.665 min, (Method B); MS (ESI, Pos.): m/z 491.1 (M+H)+;

¹H NMR (CD₃OD) δ: 7.95-7.88, 7.81-7.72, 7.53, 7.45-7.35, 4.71, 4.62-4.59, 3.76-3.72, 3.69-3.65, 3.59, 3.40-3.35, 2.82, 2.63-2.50, 1.78-1.34;

SFC Retention Time: 2.95 min (Method: E1).

### Example 106: (3S,4R,5R,6S)-1-(6-{[2-(3,4-difluorophenyl)-1,3-thiazol-4-yl]methoxy}-5-fluorohexyl)-3,4,5,6-azepanetetrol (Isomer 2)

LCMS: retention time 1.658 min, (Method B); MS (ESI, Pos.): m/z 491.1 (M+H)+;

¹H NMR (CD₃OD) δ: 7.95-7.88, 7.80-7.75, 7.53, 7.41-7.33, 4.71, 4.62-4.59, 3.76-3.72, 3.69-3.66, 3.61-3.54, 3.39, 2.81, 2.59-2.49, 1.75-1.49;

SFC Retention Time: 2.858 min (Method: E1).

### Example 107: (3S,4R,5R,6S)-1-{(5S)-6-[(4-chlorobenzyl)oxy]-5-fluorohexyl}-3,4,5,6-azepanetetrol (Isomer 1)

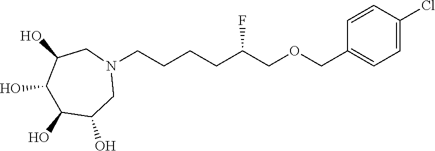 [SMILES: O[C@H]1[C@H](O)[C@@H](O)CN(CCCC[C@H](F)COCc2ccc(Cl)cc2)C[C@@H]1O]

LCMS: retention time 0.694 min, (Method A); MS (ESI, Pos.): m/z 406.1 (M+H)+;

¹H NMR (CDCl₃) δ: 7.36-7.29, 4.78-4.60, 4.55, 3.71, 3.63-3.51, 2.91, 2.69-2.53, 1.59-1.27;

SFC Retention Time: 2.686 min (Method: F4).

### Example 108: (3S,4R,5R,6S)-1-{(5R)-6-[(4-chlorobenzyl)oxy]-5-fluorohexyl}-3,4,5,6-azepanetetrol (Isomer 2)

LCMS: retention time 0.696 min, (Method A); MS (ESI, Pos.): m/z 406.1 (M+H)+;

¹H NMR (CDCl₃) δ: 7.37-7.29, 4.77-4.60, 4.56, 3.71, 3.64-3.50, 2.92, 2.69-2.52, 1.59-1.37;

SFC Retention Time: 2.973 min (Method: F4).

### Example 109: (3S,4R,5R,6S)-1-{6-[(3,4-difluorobenzyl)oxy]-5-fluorohexyl}-3,4,5,6-azepanetetrol (Isomer 1)

LCMS: retention time 0.676 min, (Method A); MS (ESI, Pos.): m/z 430.1 (M+Na)+;

¹H NMR (CD₃OD) δ: 7.31-7.18, 7.16-7.08, 4.74-4.60, 4.53, 3.67-3.62, 3.62-3.51, 3.44-3.35, 2.82, 2.62-2.45, 1.81-1.22;

SFC Retention Time: 2.01 min (Method: F4).

### Example 110: (3S,4R,5R,6S)-1-{6-[(3,4-difluorobenzyl)oxy]-5-fluorohexyl}-3,4,5,6-azepanetetrol (Isomer 2)

LCMS: retention time 0.672 min, (Method A); MS (ESI, Pos.): m/z 430 (M+Na)+;

¹H NMR (CD₃OD) δ: 7.31-7.19, 7.17-7.09, 4.74-4.60, 4.53, 3.66-3.62, 3.60-3.52, 3.43-3.35, 2.80, 2.60-2.44, 1.73-1.39;

SFC Retention Time: 2.15 min (Method: F4).

### Example 111: (3S,4R,5R,6S)-1-{6-[(3-ethoxybenzyl)oxy]-5-fluorohexyl}-3,4,5,6-azepanetetrol (Isomer 1)

LCMS: retention time 1.536 min, (Method B); MS (ESI, Pos.): m/z 416.2 (M+H)+;

¹H NMR (CD₃OD) δ: 7.24, 6.94-6.87, 6.85-6.79, 4.72-4.50, 4.53, 4.03, 3.63-3.53, 3.41-3.37, 2.80, 2.59-2.47, 1.72-1.43, 1.38;

SFC Retention Time: 4.762 min (Method: G1).

### Example 112: (3S,4R,5R,6S)-1-{6-[(3-ethoxybenzyl)oxy]-5-fluorohexyl}-3,4,5,6-azepanetetrol (Isomer 2)

LCMS: retention time 1.534 min, (Method B); MS (ESI, Pos.): m/z 416.1 (M+H)+;

¹H NMR (CD₃OD) δ: 7.24, 6.94-6.87, 6.85-6.79, 4.73-4.50, 4.53, 4.03, 3.65-3.52, 3.39, 2.81, 2.60-2.46, 1.74-1.42, 1.38;

SFC Retention Time: 4.577 min (Method: G1).

### Example 113: (3S,4R,5R,6S)-1-{6-[(2,4-difluorobenzyl)oxy]-5-fluorohexyl}-3,4,5,6-azepanetetrol (Isomer 1)

LCMS: retention time 1.461 min, (Method B); MS (ESI, Pos.): m/z 408.1 (M+H)+;

¹H NMR (CD₃OD) δ: 7.53-7.42, 7.00-6.91, 4.70-4.60, 4.58, 3.68-3.62, 3.61-3.52, 3.42-3.35, 2.80, 2.60-2.47, 1.75-1.58, 1.56-1.33;

SFC Retention Time: 4.784 min (Method: F5).

### Example 114: (3S,4R,5R,6S)-1-{6-[(2,4-difluorobenzyl)oxy]-5-fluorohexyl}-3,4,5,6-azepanetetrol (Isomer 2)

LCMS: retention time 1.47 min, (Method B); MS (ESI, Pos.): m/z 408.1 (M+H)+;

¹H NMR (CD₃OD) δ: 7.53-7.43, 7.03-6.91, 4.70-4.60, 4.59, 3.68-3.63, 3.61-3.53, 3.42-3.36, 2.80, 2.60-2.48, 1.74-1.58, 1.57-1.38;

SFC Retention Time: 5.027 min (Method: F5).

### Example 115: (3S,4R,5R,6S)-1-{5-fluoro-6-[(2-propylbenzyl)oxy]hexyl}-3,4,5,6-azepanetetrol (Isomer 1)

LCMS: retention time 0.719 min, (Method A); MS (ESI, Pos.): m/z 414.1 (M+H)+;

¹H NMR (CD₃OD) δ: 7.31, 7.26-7.12, 4.72-4.50, 3.69-3.51, 3.43-3.35, 2.81, 2.70-2.62, 2.59-2.45, 1.74-1.32, 0.98;

SFC Retention Time: 2.744 min (Method: E1).

### Example 116: (3S,4R,5R,6S)-1-{5-fluoro-6-[(2-propylbenzyl)oxy]hexyl}-3,4,5,6-azepanetetrol (Isomer 2)

LCMS: retention time 0.721 min, (Method A); MS (ESI, Pos.): m/z 414.1 (M+H)+;

¹H NMR (CD₃OD) δ: 7.31, 7.25-7.10, 4.71-4.52, 3.68-3.51, 3.43-3.35, 2.81, 2.66, 2.59-2.45, 1.74-1.34, 0.98;

SFC Retention Time: 2.997 min (Method: E1).

### Example 117: (3S,4R,5R,6S)-1-{6-[(4-ethyl-2-methylbenzyl)oxy]-5-fluorohexyl}-3,4,5,6-azepanetetrol (Isomer 1)

LCMS: retention time 0.725 min, (Method A); MS (ESI, Pos.): m/z 414.1 (M+H)+;

¹H NMR (CD₃OD) δ: 7.18, 7.05-6.95, 4.72-4.63, 4.53, 3.62-3.51, 3.43-3.36, 2.80, 2.63-2.49, 2.32, 1.74-1.60, 1.54-1.34, 1.21;

SFC Retention Time: 5.967 min (Method: B4).

### Example 118: (3S,4R,5R,6S)-1-{6-[(4-ethyl-2-methylbenzyl)oxy]-5-fluorohexyl}-3,4,5,6-azepanetetrol (Isomer 2)

LCMS: retention time 0.73 min, (Method A); MS (ESI, Pos.): m/z 414.1 (M+H)+;

¹H NMR (CD₃OD) δ: 7.18, 7.05-6.96, 4.71-4.61, 4.53, 3.66-3.52, 3.45-3.36, 2.82, 2.64-2.49, 2.32, 1.72-1.60, 1.55-1.36, 1.21;

SFC Retention Time: 6.48 min (Method: B4).

### Example 119: (3S,4R,5R,6S)-1-(5-fluoro-6-{[2-(4-methylphenyl)-1,3-oxazol-4-yl]methoxy}hexyl)-3,4,5,6-azepanetetrol (Isomer 1)

LCMS: retention time 0.702 min, (Method A); MS (ESI, Pos.): m/z 475.2 (M+Na)+;

¹H NMR (CD₃OD) δ: 7.92, 7.91, 7.33, 4.73-4.56, 4.54, 3.72-3.67, 3.65-3.62, 3.62-3.54, 3.44-3.37, 2.82, 2.62-2.48, 2.41, 1.70-1.33;

SFC Retention Time: 4.729 min (Method: F1).

### Example 120: (3S,4R,5R,6S)-1-(5-fluoro-6-{[2-(4-methylphenyl)-1,3-oxazol-4-yl]methoxy}hexyl)-3,4,5,6-azepanetetrol (Isomer 2)

LCMS: retention time 0.697 min, (Method A); MS (ESI, Pos.): m/z 475.1 (M+Na)+;

¹H NMR (CD₃OD) δ: 7.92, 7.91, 7.33, 4.73-4.56, 4.55, 3.73-3.63, 3.62-3.54, 3.45-3.38, 2.83, 2.63-2.51, 2.41, 1.70-1.35;

SFC Retention Time: 6.14 min (Method: F1).

### Example 121: (3S,4R,5R,6S)-1-(5-fluoro-6-{[2-(4-fluorophenyl)-1,3-oxazol-4-yl]methoxy}hexyl)-3,4,5,6-azepanetetrol (Isomer 1)

LCMS: retention time 0.677 min, (Method A); MS (ESI, Pos.): m/z 457.1 (M+H)+;

¹H NMR (CD₃OD) δ: 8.07, 7.95, 7.28-7.23, 4.74-4.60, 4.57, 3.73-3.68, 3.66-3.62, 3.60-3.53, 3.41-3.36, 2.79, 2.58-2.44, 1.74-1.60, 1.57-1.46;

SFC Retention Time: 5.91 min (Method: F3).

### Example 122: (3S,4R,5R,6S)-1-(5-fluoro-6-{[2-(4-fluorophenyl)-1,3-oxazol-4-yl]methoxy}hexyl)-3,4,5,6-azepanetetrol (Isomer 2)

LCMS: retention time 0.689 min, (Method A); MS (ESI, Pos.): m/z 457.1 (M+H)+;

¹H NMR (CD₃OD) δ: 8.08, 7.95, 7.28-7.23, 4.73-4.60, 4.56, 3.72-3.68, 3.66-3.62, 3.59-3.52, 3.40-3.36, 2.79, 2.57-2.47, 1.73-1.63, 1.55-1.32;

SFC Retention Time: 6.763 min (Method: F3).

### Example 123: (3S,4R,5R,6S)-1-(5-fluoro-6-{[2-(3-fluorophenyl)-1,3-oxazol-4-yl]methoxy}hexyl)-3,4,5,6-azepanetetrol (Isomer 1)

LCMS: retention time 0.689 min, (Method A); MS (ESI, Pos.): m/z 457.1 (M+H)+;

¹H NMR (CD₃OD) δ: 7.98, 7.86, 7.79-7.70, 7.59-7.50, 7.31-7.22, 4.75-4.60, 4.58, 3.73-3.69, 3.67-3.63, 3.60-3.51, 3.41-3.37, 2.80, 2.58-2.48, 1.75-1.58, 1.56-1.34;

SFC Retention Time: 1.704 min (Method: F2).

### Example 124: (3S,4R,5R,6S)-1-(5-fluoro-6-{[2-(3-fluorophenyl)-1,3-oxazol-4-yl]methoxy}hexyl)-3,4,5,6-azepanetetrol (Isomer 2)

LCMS: retention time 0.687 min, (Method A); MS (ESI, Pos.): m/z 457.1 (M+H)+;

¹H NMR (CD₃OD) δ: 7.98, 7.86, 7.79-7.73, 7.59-7.50, 7.30-7.22, 4.73-4.60, 4.58, 3.73-3.69, 3.66-3.63, 3.60-3.52, 3.40-3.37, 2.80, 2.57-2.47, 1.74-1.62, 1.57-1.36;

SFC Retention Time: 2.442 min (Method: F2).

### Example 125: (3S,4R,5R,6S)-1-{6-[(4-butylbenzyl)oxy]-5-fluorohexyl}-3,4,5,6-azepanetetrol (Isomer 1)

LCMS: retention time 0.757 min, (Method A); MS (ESI, Pos.): m/z 428.2 (M+H)+;

¹H NMR (CD₃OD) δ: 7.25, 7.17, 4.72-4.63, 4.52, 3.63-3.51, 3.42-3.35, 2.80, 2.64-2.47, 1.71-1.49, 1.47-1.27, 0.94;

SFC Retention Time: 0.8 min (Method: B7).

### Example 126: (3S,4R,5R,6S)-1-{6-[(4-butylbenzyl)oxy]-5-fluorohexyl}-3,4,5,6-azepanetetrol (Isomer 2)

LCMS: retention time 4.203 min, (Method C); MS (ESI, Pos.): m/z 428.2 (M+H)+;

¹H NMR (CD₃OD) δ: 7.24, 7.16, 4.73-4.53, 4.52, 3.69-3.53, 3.46-3.39, 2.88, 2.70-2.55, 1.72-1.51, 1.48-1.29, 0.94;

SFC Retention Time: 1.292 min (Method: B7).

### Example 127: (3S,4R,5R,6S)-1-{5-fluoro-6-[(2-fluoro-4-propylbenzyl)oxy]hexyl}-3,4,5,6-azepanetetrol (Isomer 1)

LCMS: retention time 1.8 min, (Method B); MS (ESI, Pos.): m/z 432.2 (M+H)+;

¹H NMR (CD₃OD) δ: 7.32, 7.00, 6.94, 4.73-4.55, 4.58, 3.65-3.50, 3.50-3.52, 3.41-3.37, 2.89-2.76, 2.63-2.48, 1.67-1.38, 0.94;

SFC Retention Time: 3.093 min (Method: A3).

### Example 128: (3S,4R,5R,6S)-1-{5-fluoro-6-[(2-fluoro-4-propylbenzyl)oxy]hexyl}-3,4,5,6-azepanetetrol (Isomer 2)

LCMS: retention time 1.793 min, (Method B); MS (ESI, Pos.): m/z 432.1 (M+H)+;

¹H NMR (CD₃OD) δ: 7.32, 7.00, 6.93, 4.72-4.55, 4.58, 3.65-3.52, 3.44-3.36, 2.86-2.75, 2.63-2.45, 1.71-1.55, 1.55-1.35, 0.94;

SFC Retention Time: 3.485 min (Method: A3).

### Example 129: (3S,4R,5R,6S)-1-{5-fluoro-6-[(2-methyl-4-propylbenzyl)oxy]hexyl}-3,4,5,6-azepanetetrol (Isomer 1)

LCMS: retention time 0.755 min, (Method A); MS (ESI, Pos.): m/z 428.2 (M+H)+;

¹H NMR (CD₃OD) δ: 7.21-7.15, 7.02-6.94, 4.75-4.67, 4.58-4.49, 3.71-3.50, 3.46-3.36, 2.84, 2.65-2.49, 2.32, 1.73-1.30, 0.92;

SFC Retention Time: 5.982 min (Method: B4).

### Example 130: (3S,4R,5R,6S)-1-{5-fluoro-6-[(2-methyl-4-propylbenzyl)oxy]hexyl}-3,4,5,6-azepanetetrol (Isomer 2)

LCMS: retention time 0.75 min, (Method A); MS (ESI, Pos.): m/z 428.1 (M+H)+;

¹H NMR (CD₃OD) δ: 7.17, 7.03-6.91, 4.70-4.62, 4.57-4.49, 3.68-3.51, 3.45-3.37, 2.85, 2.68-2.47, 2.31, 1.68-1.33, 0.92;

SFC Retention Time: 6.677 min (Method: B4).

### Example 131: (3S,4R,5R,6S)-1-{6-[(2,6-difluoro-4-propylbenzyl)oxy]-5-fluorohexyl}-3,4,5,6-azepanetetrol (Isomer 1)

LCMS: retention time 0.749 min, (Method A); MS (ESI, Pos.): m/z 450.1 (M+H)+;

¹H NMR (CD₃OD) δ: 6.84, 4.60, 4.63-4.47, 3.67-3.51, 3.45-3.35, 2.83, 2.64-2.46, 1.72-1.32, 0.94;

SFC Retention Time: 6.545 min (Method: A3).

### Example 132: (3S,4R,5R,6S)-1-{6-[(2,6-difluoro-4-propylbenzyl)oxy]-5-fluorohexyl}-3,4,5,6-azepanetetrol (Isomer 2)

LCMS: retention time 0.753 min, (Method A); MS (ESI, Pos.): m/z 450.1 (M+H)+;

¹H NMR (CD₃OD) δ: 6.85, 4.61, 4.63-4.48, 3.68-3.52, 3.44-3.36, 2.83, 2.65-2.50, 1.70-1.57, 1.55-1.31, 0.95;

SFC Retention Time: 6.945 min (Method: A3).

### Example 133: (3S,4R,5R,6S)-1-{6-[(2,4-dimethylbenzyl)oxy]-5-fluorohexyl}-3,4,5,6-azepanetetrol (Isomer 1)

LCMS: retention time 1.601 min, (Method B); MS (ESI, Pos.): m/z 400.2 (M+H)+;

¹H NMR (CD₃OD) δ: 7.15, 7.00, 6.96, 4.69-4.50, 3.63-3.51, 3.40-3.36, 2.80, 2.59-2.42, 2.30, 2.28, 1.73-1.29;

SFC Retention Time: 4.069 min (Method: H₁).

### Example 134: (3S,4R,5R,6S)-1-{6-[(2,4-dimethylbenzyl)oxy]-5-fluorohexyl}-3,4,5,6-azepanetetrol (Isomer 2)

LCMS: retention time 1.597 min, (Method B); MS (ESI, Pos.): m/z 400.2 (M+H)+;

¹H NMR (CD₃OD) δ: 7.15, 7.00, 6.96, 4.70-4.52, 3.64-3.56, 3.56-3.53, 3.46-3.37, 2.84, 2.59-2.42, 2.30, 1.76-1.32;

SFC Retention Time: 4.58 min (Method: H₁).

### Reference Example 27: 1-[(4-methylbenzyl)oxy]-6-[(triisopropylsilyl)oxy]-2-hexanol

To a solution of (4-methylphenyl)methanol (50 g) in t-BuOH (250 mL) were added 15% NaOHaq. (250 mL) and triisopropyl(4-(oxiran-2-yl)butoxy)silane (123 g) and TBAI (66 g) at 20° C. The reaction was stirred at 90° C. for 15 h. The mixture was poured into water and extracted with EtOAc. The organic phase was concentrated and purified by flash column with silica gel (PE to 5% EtOAc in PE) to give the title compound (79.5 g).

TLC R_{f}=0.40 (PE/EtOAc=5/1)

### Reference Example 28: 6-[(4-methylbenzyl)oxy]-1,5-hexanediol

To a solution of the compound prepared in Reference Example 27 (79.5 g) in THF (250 mL) was added 1M TBAF in THF (292 mL) at 20° C. The reaction was stirred at 20° C. for 1 h. The reaction mixture was concentrated and the residue was purified by flash column with silica gel (PE to 35% EtOAc in PE) to give the title compound (44.7 g).

TLC R_{f}=0.20 (PE/EtOAc=2/1)

### Reference Example 29: 5-hydroxy-6-[(4-methylbenzyl]oxy]hexyl 4-methylbenzenesulfonate

To a solution of the compound prepared in Reference Example 28 (44.7 g) in DCM (400 mL) was added TsCl (36.3 g) and TEA (52.3 mL) at 0° C. The mixture was stirred at 20° C. for 16 h. The reaction mixture was concentrated and the residue was purified by flash column with silica gel (PE to 20% EtOAc in PE) to give the title compound (45.4 g).

TLC R_{f}=0.60 (PE/EtOAc=3/1)

### Reference Example 30: 5-fluoro-6-[(4-methylbenzyl]oxy]hexyl 4-methylbenzenesulfonate

To a solution of the compound prepared in Reference Example 29 (45.4 g) in THF (450 mL) was added PBSF (39.9 mL), hydrogen fluoride in triethylamine (37.7 mL) and TEA (53.1 mL) at 0° C. The reaction was stirred at 20° C. for 16 h. The reaction mixture was poured into water and extracted with EtOAc. The combined organic layers were dried over Na₂SO₄ and concentrated. The residue was purified by flash column with silica gel (PE to 4% EtOAc in PE) to afford the title compound (22.7 g).

TLC R_{f}=0.50 (PE/EtOAc=5/1)

### Example 135: (3S,4R,5R,6S)-1-{(5S)-5-fluoro-6-[(4-methylbenzyl)oxy]hexyl}-3,4,5,6-azepanetetrol (Isomer 1)

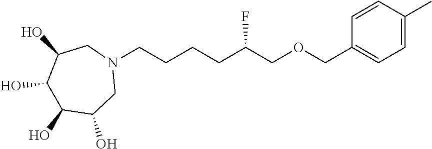 [SMILES: Cc1ccc(COC[C@@H](F)CCCCN2C[C@H](O)[C@@H](O)[C@H](O)[C@@H](O)C2)cc1]

### Example 136: (3S,4R,5R,6S)-1-{(5R)-5-fluoro-6-[(4-methylbenzyl)oxy]hexyl}-3,4,5,6-azepanetetrol (Isomer 2)

To a solution of (3S,4R,5R,6S)-azepane-3,4,5,6-tetrol hydrochloride salt (700 mg) in DMF (5 mL) were added DIEA (5.5 mL), KI (53 mg) and the compound prepared in Reference Example 30 (1.25 g) at 20~25° C. The reaction mixture was stirred at 80° C. for 16 h. The mixture was treated with sat. NaHCO₃ aq. and extracted by EtOAc. The combined organic layers were dried over Na₂SO₄ and concentrated to dryness. The residue was purified by C-18 reversed phase column (10~30% MeCN in water) and chiral SFC separation to obtain isomer 1 (403 mg) and isomer 2 (358 mg).

Example 135 (isomer 1): LCMS: retention time 1.514 min, (Method B); MS (ESI, Pos.): m/z 386.1 (M+H)+;

¹H NMR (CD₃OD) δ: 7.22, 7.16, 4.70-4.51, 4.50, 3.61-3.50, 3.41-3.37, 2.80, 2.60-2.48, 2.33, 1.75-1.58, 1.57-1.31;

SFC Retention Time: 3.123 min (Method: B3).

Example 136 (isomer 2): LCMS: retention time 1.495 min, (Method B); MS (ESI, Pos.): m/z 386.2 (M+H)+;

¹H NMR (CD₃OD) δ: 7.22, 7.16, 4.70-4.51, 4.50, 3.64-3.55, 3.44-3.37, 2.83, 2.64-2.48, 2.33, 1.73-1.57, 1.56-1.32;

SFC Retention Time: 3.884 min (Method: B3).

### Reference Example 31: methyl 2-(3,5-difluorophenyl)-1,3-oxazole-4-carboxylate

To the mixture of methyl 2-amino-3-hydroxypropanoate hydrochloride (CAS No. 5680-80-8, 500 mg) and 3,5-difluorobenzaldehyde (CAS No. 32085-88-4, 460 mg) in DMA (10 mL) was added K₂CO₃ (888 mg), the reaction was stirred at 25° C. for 16 h. To the reaction mixture was added BrCCl₃ (1.9 g) and DBU (1.4 mL) at 0° C., the reaction was stirred at 25° C. for 16 h. The mixture was purified by flash column with silica gel (EtOAc in PE=0~40%,) to give the title compound (390 mg).

TLC R_{f}=0.50 (EtOAc/PE=1/3).

### Reference Example 32: [2-(3,5-difluorophenyl)-1,3-oxazol-4-yl]methanol

To a solution of the compound prepared in Reference Example 31 (100 mg) in THF (3 mL) was added LiAlH₄ (20 mg) at 0° C., the reaction was stirred at 0° C. for 3 h to give a yellow solution. The reaction mixture was quenched by water, 15% NaOHaq. and water. The mixture was dried over Na₂SO₄ and filtered. The filtrate was concentrated and purified by flash column with silica gel (EtOAc in PE=0~50%,) to give the title compound (74 mg).

TLC R_{f}=0.30 (EtOAc/PE=1/3).

### Example 137 and 138

Example 137 and 138 were synthesized according to procedures analogous to Reference Example 23, 24, 25, 26, Example 71 and 72 using the corresponding alcohol prepared in Reference example 32 instead of (4-ethylphenyl)methanol.

### Example 137: (3S,4R,5R,6S)-1-(6-{[2-(3,5-difluorophenyl)-1,3-oxazol-4-yl]methoxy}-5-fluorohexyl)-3,4,5,6-azepanetetrol (Isomer 1)

LCMS: retention time 0.778 min, (Method A); MS (ESI, Pos.): m/z 475.2 (M+H)+;

¹H NMR (CD₃OD) δ: 8.00, 7.64-7.56, 7.22-7.06, 4.74-4.69, 4.59, 3.77-3.51, 3.47-3.35, 2.83, 2.69-2.44, 1.76-1.35;

SFC Retention Time: 2.37 min (Method: F2).

### Example 138: (3S,4R,5R,6S)-1-(6-{[2-(3,5-difluorophenyl)-1,3-oxazol-4-yl]methoxy}-5-fluorohexyl)-3,4,5,6-azepanetetrol (Isomer 2)

LCMS: retention time 0.781 min, (Method A); MS (ESI, Pos.): m/z 475.2 (M+H)+;

¹H NMR (CD₃OD) δ: 8.00, 7.66-7.49, 7.19-7.05, 4.74-4.65, 4.59, 3.76-3.49, 3.42-3.34, 2.79, 2.58-2.43, 1.75-1.34;

SFC Retention Time: 3.083 min (Method: F2).

### Reference Example 33: methyl 6-(3,5-difluorophenyl)-3-pyridazinecarboxylate

To a solution of methyl 6-chloropyridazine-3-carboxylate (CAS No. 65202-50-8, 25 g) and (3,5-difluorophenyl)boronic acid (32 g) in DMF (250 mL) was added K₃PO₄ (40 g) and Pd(dppf)Cl₂ (1.1 g) under N₂. The mixture was stirred at 70° C. for 12 h under N₂. The mixture was cooled to 20° C., poured into water and filtered. The filter cake was washed with water. The filtrate was triturated with MeOH and dried in vacuo to give the title compound (10 g), which was used in the next step without any further purification.

### Reference Example 34: [6-(3,5-difluorophenyl)-3-pyridazinyl]methanol

To a mixture of the compound prepared in Reference Example 33 (4.2 g) in THF (80 mL) was added CaCl₂ (1.9 g) and NaBH₄ (0.95 g) at 0° C. The reaction mixture was stirred at 10° C. for 0.5 h and followed by addition of MeOH (4 mL) dropwise at 0° C. The mixture was allowed to stirred at 10° C. for 2 h to give a yellow solution. The reaction mixture was acidified with 1M HClaq. to pH=8. Water and EtOAc was added to the mixture. The resulting mixture was filtered. The filtrate was extracted with EtOAc. The combined organic phase were washed brine, dried over anhydrous Na₂SO₄, filtered and concentrated in vacuo. The residue was purified by flash column with silica gel (0%~40% EtOAc in PE,) to give the title compound (2.3 g).

TLC R_{f}=0.10 (PE/EtOAc=2/1).

### Example 139 and 140

Example 139 and 140 were synthesized according to procedures analogous to Reference Example 23, 24, 25, 26, Example 71 and 72 using the corresponding alcohol prepared in Reference example 34 instead of (4-ethylphenyl)methanol.

### Example 139: (3S,4R,5R,6S)-1-[(5S)-6-{[6-(3,5-difluorophenyl)-3-pyridazinyl]methoxy}-5-fluorohexyl]-3,4,5,6-azepanetetrol (Isomer 1)

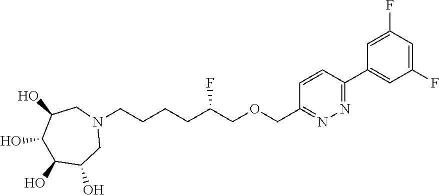 [SMILES: O[C@H]1[C@H](O)[C@@H](O)CN(CCCC[C@H](F)COCc2ccc(-c3cc(F)cc(F)c3)nn2)C[C@@H]1O]

LCMS: retention time 0.658 min, (Method A); MS (ESI, Pos.): m/z 486.1 (M+H)+;

¹H NMR (CD₃OD) δ: 8.25, 7.92, 7.82-7.71, 7.19-7.08, 4.94, 4.80-4.63, 3.85-3.70, 3.60-3.52, 3.43-3.36, 2.81, 2.59-2.49, 1.76-1.42;

SFC Retention Time: 1.838 min (Method: F6).

### Example 140: (3S,4R,5R,6S)-1-[(5R)-6-{[6-(3,5-difluorophenyl)-3-pyridazinyl]methoxy}-5-fluorohexyl]-3,4,5,6-azepanetetrol (Isomer 2)

LCMS: retention time 0.662 min, (Method A); MS (ESI, Pos.): m/z 486.1 (M+H)+;

¹H NMR (CD₃OD) δ: 8.25, 7.93, 7.82-7.71, 7.19-7.08, 4.94, 4.79-4.63, 3.89-3.68, 3.60-3.52, 3.43-3.36, 2.81, 2.62-2.44, 1.76-1.35;

SFC Retention Time: 3.359 min (Method: F6).

### Reference Example 35: (3E)-6-(4-biphenylylmethoxy)-3-hexen-1-ol

To a solution of 4-(bromomethyl)-1,1′-biphenyl (CAS No. 2567-29-5, 250 mg) in THF (3 mL) and H₂O (0.6 mL) was added TBAB (33 mg), sat. NaOH aq. (0.28 mL) and (E)-hex-3-ene-1,6-diol (CAS No. 71655-17-9, 141 mg) at 20-25° C., the reaction was stirred at 60° C. for 15 h to give a yellow solution. The reaction mixture was poured into water and extracted with EtOAc. The combined organic phases were dried over Na₂SO₄, filtered and concentrated to dryness. The residue was directly purified by flash column with silica gel (PE to 15% EtOAc in PE) to afford the title compound (130 mg).

¹H NMR (CDCl₃) δ: 7.62-7.56, 7.47-7.38, 7.37-7.32, 5.65-5.55, 5.54-5.45, 4.56, 3.64, 3.54, 2.42-2.34, 2.29.

### Example 141: (3S,4R,5R,6S)-1-[(3E)-6-(4-biphenylylmethoxy)-3-hexen-1-yl]-3,4,5,6-azepanetetrol

Example 141 was synthesized according to procedures analogous to Reference Example 7 and Example 69 using the corresponding alcohol prepared in Reference example 35 instead of the compound prepared in Reference Example 6.

LCMS: retention time 0.725 min, (Method A); MS (ESI, Pos.): m/z 428.1 (M+H)+;

¹H NMR (DMSO-D₆) δ: 7.68-7.62, 7.50-7.43, 7.40, 7.38-7.33, 5.49-5.44, 4.68, 4.50, 3.45, 3.40-3.34, 3.21-3.16, 2.67, 2.48-2.38, 2.29-2.21, 2.14-2.05.

### Example 142 to 146

Example 142 to 146 were synthesized according to procedures analogous to Reference Example 7 and Example 69 using the corresponding alcohol instead of the compound prepared in Reference Example 6.

### Example 142: (3S,4R,5R,6S)-1-[(3E)-6-{[2-(2,3,4-trifluorophenyl)-1,3-thiazol-4-yl]methoxy}-3-hexen-1-yl]-3,4,5,6-azepanetetrol

LCMS: retention time 1.705 min, (Method B); MS (ESI, Pos.): m/z 489.1 (M+H)+;

¹H NMR (CD₃OD) δ: 8.16-7.93, 7.63, 7.36-7.21, 5.59-5.46, 4.70, 3.63-3.52, 3.41-3.34, 2.81, 2.59-2.51, 2.36-2.29, 2.23-2.15.

### Example 143: (3S,4R,5R,6S)-1-{(3E)-6-[(4-propylbenzyl)oxy]-3-hexen-1-yl}-3,4,5,6-azepanetetrol

LCMS: retention time 1.784 min, (Method B); MS (ESI, Pos.): m/z 394.4 (M+H)+;

¹H NMR (CD₃OD) δ: 7.24, 7.15, 5.54-5.48, 4.46, 3.59-3.54, 3.48, 3.41-3.37, 2.83, 2.61-2.52, 2.31-2.23, 2.23-2.17, 1.68-1.54, 0.93.

### Example 144: (3S,4R,5R,6S)-1-[(3E)-6-{[2-(3,4-difluorophenyl)-1,3-thiazol-4-yl]methoxy}-3-hexen-1-yl]-3,4,5,6-azepanetetrol

LCMS: retention time 1.663 min, (Method B); MS (ESI, Pos.): m/z 471.1 (M+H)+;

¹H NMR (CD₃OD) δ: 7.90-7.86, 7.81-7.72, 7.49, 7.45-7.35, 5.60-5.45, 4.65, 3.65-3.51, 3.42-3.35, 2.82, 2.59-2.52, 2.37-2.27, 2.25-2.14.

### Example 145: (3S,4R,5R,6S)-1-[(3E)-6-{[2-(3,5-difluorophenyl)-1,3-thiazol-4-yl]methoxy}-3-hexen-1-yl]-3,4,5,6-azepanetetrol

LCMS: retention time 1.671 min, (Method B); MS (ESI, Pos.): m/z 471.2 (M+H)+;

¹H NMR (CD₃OD) δ: 7.70-7.46, 7.19-6.97, 5.57-5.48, 4.66, 3.64-3.53, 3.40-3.37, 2.82, 2.62-2.50, 2.33, 2.25-2.15.

### Example 146: (3S,4R,5R,6S)-1-[(3E)-6-{[2-(4-fluorophenyl)-1,3-thiazol-4-yl]methoxy}-3-hexen-1-yl]-3,4,5,6-azepanetetrol

LCMS: retention time 0.68 min, (Method A); MS (ESI, Pos.): m/z 453.1 (M+H)+;

¹H NMR (DMSO-D₆) δ: 7.98, 7.59, 7.34, 5.46, 4.67, 4.58, 4.49, 3.51, 3.36, 3.20-3.15, 2.66, 2.48-2.37, 2.25, 2.09.

### Reference Example 36: 6-{[2-(2-bromo-1,3-thiazol-4-yl)-2-propanyl]oxy}-1-hexanol

To a solution of 2-(2-bromo-1,3-thiazol-4-yl)-2-propanol (CAS No. 2228788-36-9, 180 mg) in DCM (3 mL) were added TsOH (167 mg) and 1,6-hexanediol (0.44 mL). The mixture was stirred at 50° C. for 16 h. The reaction mixture was purified by silica gel column chromatography (PE to 18% EtOAc in PE) to give the title compound (48 mg).

TLC R_{f}=0.2 (PE/EtOAc=3/1).

### Reference Example 37: 6-({2-[2-(1-propyn-1-yl)-1,3-thiazol-4-yl]-2-propanyl}oxy)-1-hexanol

To a solution of the compound prepared in Reference Example 36 (48 mg) in THF (1 mL) was added TEA (0.041 mL), followed by CuI (1.4 mg) and Pd(PPh₃)₂Cl₂ (5.2 mg). After stirring 5 min at 20° C., 1-propyne (1 mol/L in THF, 0.89 mL) was added dropwise to the reaction mixture. The reaction mixture was stirred at 50° C. for 16 h. The reaction mixture was diluted with EtOAc (10 mL) and filtered. The filtrate was concentrated. The residue was purified by silica gel column chromatography (PE to 20% EtOAc in PE) to give the title compound (25 mg).

TLC R_{f}=0.2 (PE/EtOAc=2/1).

### Example 147: (3S,4R,5R,6S)-1-[6-({2-[2-(1-propyn-1-yl)-1,3-thiazol-4-yl]-2-propanyl}oxy)hexyl]-3,4,5,6-azepanetetrol

Example 147 was synthesized according to procedures analogous to Reference Example 16 and Example 69 using the compound prepared in Reference Example 37 instead of the compound prepared in Reference Example 7.

LCMS: retention time 0.665 min, (Method A); MS (ESI, Pos.): m/z 427.1 (M+H)+;

¹H NMR (CD₃OD) δ: 7.35, 3.59, 3.40, 3.23, 2.88-2.78, 2.63-2.47, 2.13, 1.55, 1.53-1.44, 1.39-1.23.

### Reference Example 38: ethyl [2-(4-chloro-3-fluorophenyl)-1,3-oxazol-4-yl]acetate

A mixture of 4-chloro-3-fluorobenzamide (CAS No. 874781-07-4, 1.0 g) and ethyl 4-chloro-3-oxo-butanoate (CAS No. 638-07-3, 1.9 g) was stirred at 140° C. for 2 h. The reaction mixture was diluted with H₂O and extracted with EtOAc. The combined organic layers were dried over Na₂SO₄ and concentrated in vacuum to give the residue. The residue was purified by flash column with silica gel (5% EtOAc in PE) to give the title compound (760 mg).

¹H NMR (CDCl₃) δ: 7.83-7.70, 7.54-7.41, 4.27-4.18, 3.68, 1.33-1.27.

### Reference Example 39: 2-[2-(4-chloro-3-fluorophenyl)-1,3-oxazol-4-yl]ethanol

To a solution of the compound prepared in Reference Example 38 (400 mg) in THF (4 mL) was added LiAlH₄ (64 mg) at 0° C. The mixture was stirred at 20° C. for 1 h. The reaction mixture was quenched by water, and 15% NaOHaq. The mixture was stirred for 10 min. The suspension was diluted with EtOAc filtered and the filter cake was washed with EtOAc. The filtrate was dried over anhydrous Na₂SO₄, filtered and concentrated in vacuum to give the title compound (320 mg).

ESI/MS: 241.8 [M+H]⁺.

### Reference Example 40: 2-(4-chloro-3-fluorophenyl)-4-[2-(4-iodobutoxy)ethyl]-1,3-oxazole

To a solution of the compound prepared in Reference Example 39 (50 mg) in THF (0.50 mL) was added 15% NaOHaq. (2.5 mL), 1,4-diiodobutane (CAS No. 628-21-7, 0.050 mL) and TBAI (67 mg) at 20° C., the reaction mixture was stirred at 50° C. for 17 h. The reaction mixture was poured into water and extracted with EtOAc. The combined organic phases were dried over Na₂SO₄, filtered and concentrated to dryness. The residue was purified by prep-TLC (PE/EtOAc=5/1, R_{f}=0.4) to afford the title compound (25 mg).

TLC R_{f}=0.40 (PE/EtOAc=5/1).

### Example 148: (3S,4R,5R,6S)-1-(4-{2-[2-(4-chloro-3-fluorophenyl)-1,3-oxazol-4-yl]ethoxy}butyl)-3,4,5,6-azepanetetrol

Example 148 was synthesized according to procedures analogous to Example 1 using the iodide prepared in Reference Example 40 instead of the compound prepared in Reference Example 1.

LCMS: retention time 0.69 min, (Method A); MS (ESI, Pos.): m/z 458.9 (M+H)+;

¹H NMR (CD₃OD) δ: 7.92-7.80, 7.69-7.62, 3.76, 3.63, 3.53, 3.44, 2.88, 2.61, 1.61.

### Example 149 to 153

Example 149 to 153 were synthesized according to procedures analogous to Reference Example 1 and Example 1 using 1,5-diiodopentane (CAS No. 628-77-3) and corresponding alcohol compounds.

### Example 149: (3S,4R,5R,6S)-1-[5-(4-biphenylylmethoxy)pentyl]-3,4,5,6-azepanetetrol

LCMS: retention time 1.734 min, (Method B); MS (ESI, Pos.): m/z 416.3 (M+H)+;

¹H NMR (CD₃OD) δ: 7.61, 7.46-7.40, 7.32, 4.55, 3.61-3.51, 3.43-3.37, 2.81, 2.59-2.48, 1.72-1.61, 1.59-1.48, 1.46-1.37.

### Example 150: (3S,4R,5R,6S)-1-(5-{[4-(4-fluorophenyl)-1,3-thiazol-2-yl]methoxy}pentyl)-3,4,5,6-azepanetetrol Hydrochloride (1:1)

LCMS: retention time 0.739 min, (Method A); MS (ESI, Pos.): m/z 441.2 (M+H)+;

¹H NMR (D₂O) δ: 7.81-7.73, 7.19, 4.85, 4.08-4.06, 3.69-3.66, 3.34, 3.23-3.18, 1.73-1.63, 1.62-1.39.

### Example 151: (3S,4R,5R,6S)-1-[5-(3-biphenylylmethoxy)pentyl]-3,4,5,6-azepanetetrol

LCMS: retention time 0.708 min, (Method A); MS (ESI, Pos.): m/z 416.1 (M+H)+;

¹H NMR (CD₃OD) δ: 7.63-7.58, 7.54, 7.47-7.40, 7.37-7.29, 4.57, 3.59, 3.54, 3.43-3.38, 2.83, 2.63-2.51, 1.70-1.61, 1.59-1.48, 1.47-1.36.

### Example 152: (3S,4R,5R,6S)-1-(5-{[2-(3,5-difluorophenyl)-1,3-thiazol-4-yl]methoxy}pentyl)-3,4,5,6-azepanetetrol

LCMS: retention time 1.632 min, (Method B); MS (ESI, Pos.): m/z 459.2 (M+H)+;

¹H NMR (CD₃OD) δ: 7.64-7.49, 7.13-7.03, 4.65, 3.64-3.53, 3.42-3.35, 2.82, 2.78, 2.58-2.48, 1.70-1.61, 1.58-1.48, 1.47-1.37.

### Example 153: (3S,4R,5R,6S)-1-[5-(2-biphenylylmethoxy)pentyl]-3,4,5,6-azepanetetrol

LCMS: retention time 1.736 min, (Method B); MS (ESI, Pos.): m/z 416.3 (M+H)+;

¹H NMR (CD₃OD) δ: 7.52-7.47, 7.44-7.39, 7.39-7.32, 7.28-7.22, 4.36, 3.60-3.54, 3.41-3.36, 2.80, 2.57-2.47, 1.58-1.43, 1.37-1.28.

### Reference Example 41: 2-[2-(3,5-difluorophenyl)-1,3-thiazol-4-yl]ethanol

Titled compound was synthesized according to procedures analogous to Reference Example 39 using ethyl 2-(2-(3,5-difluorophenyl)thiazol-4-yl)acetate (CAS No. 1501541-90-7) instead of the compound prepared in Reference Example 38.

TLC R_{f}=0.40 (PE/EtOAc=3/1).

### Example 154: (3S,4R,5R,6S)-1-(5-{2-[2-(3,5-difluorophenyl)-1,3-thiazol-4-yl]ethoxy}pentyl)-3,4,5,6-azepanetetrol

Example 154 was synthesized according to procedures analogous to Reference Example 1 and Example 1 using 1,5-diiodopentane and the compound prepared in Reference Example 41.

LCMS: retention time 0.706 min, (Method A); MS (ESI, Pos.): m/z 473 (M+H)+;

¹H NMR (DMSO-D₆) δ: 7.64-7.57, 7.51, 7.40-7.32, 4.66, 4.48, 3.71, 3.42-3.37, 3.19-3.14, 2.99, 2.61, 2.40-2.31, 1.52-1.40, 1.40-1.29, 1.28-1.17.

### Example 155 and 156

Example 155 and 156 were synthesized according to procedures analogous to Reference Example 1 and Example 1 using 1,7-diiodoheptane (CAS No. 51526-03-5) and corresponding alcohol compounds.

### Example 155: (3S,4R,5R,6S)-1-(7-{[4-(4-fluorophenyl)-1,3-thiazol-2-yl]methoxy}heptyl)-3,4,5,6-azepanetetrol

LCMS: retention time 0.781 min, (Method C); MS (ESI, Pos.): m/z 469.2 (M+H)+;

¹H NMR (CD₃OD) δ: 7.99-7.90, 7.76, 7.18-7.12, 4.82, 3.64, 3.58-3.57, 3.40-3.38, 2.85-2.72, 2.57-2.50, 1.68-1.64, 1.50-1.33.

### Example 156: (3S,4R,5R,6S)-1-(7-{[2-(4-fluorophenyl)-1,3-thiazol-4-yl]methoxy}heptyl)-3,4,5,6-azepanetetrol

LCMS: retention time 0.702 min, (Method A); MS (ESI, Pos.): m/z 469.1 (M+H)+;

¹H NMR (DMSO-D₆) δ: 7.98, 7.58, 7.34, 4.68, 4.56, 4.49, 3.50, 3.45-3.37, 3.24-3.11, 2.75-2.55, 2.47-2.26, 1.62-1.47, 1.44-1.18.

### Example 157: (3S,4R,5R,6S)-1-{4-[(4′-methyl-4-biphenylyl)methoxy]butyl}-3,4,5,6-azepanetetrol

Example 157 was synthesized according to procedures analogous to Reference Example 1 and Example 1 using 1,4-diiodobutane and corresponding alcohol compounds.

LCMS: retention time 0.723 min, (Method A); MS (ESI, Pos.): m/z 416.1 (M+H)+;

¹H NMR (CDCl₃) δ: 7.55, 7.48, 7.37, 7.23, 4.51, 3.64, 3.58-3.36, 2.85, 2.66-2.46, 2.38, 2.11-1.70, 1.68-1.48, 1.35-1.23.

### Reference Example 42: methyl 4,4-difluoro-6-hydroxyhexanoate

To a mixture of 5,5-difluorooxepan-2-one (CAS No. 35196-95-3, 1.7 g) in methanol (20 mL) was added MeONa (600 mg). The reaction mixture was stirred at 25° C. for 16 h. The reaction mixture was quenched by sat. NH₄Claq. and extracted with DCM. The combined organic phase were washed with brine, dried over anhydrous Na₂SO₄ and filtered. The filtrate was concentrated in vacuum to give the title compound (1.7 g), which was used in the next step without any further purification.

### Reference Example 43: methyl 4,4-difluoro-6-[(triisopropylsilyl)oxy]hexanoate

To a mixture of the compound prepared in Reference Example 42 (0.70 g) in DMF (10 mL) was added chlorotriisopropylsilane (0.90 mL) and imidazole (520 mg). The reaction mixture was stirred at 25° C. for 16 h to give a brown mixture. The reaction mixture was diluted with water and extracted with EtOAc. The combined organic phase was washed with water, brine, dried over anhydrous Na₂SO₄ and filtered. The filtrate was concentrated. The residue was purified by silica gel column chromatography (PE to 10% EtOAc in PE) to give the title compound (0.73 g).

TLC R_{f}=0.4 (PE/EtOAc=20/1).

### Reference Example 44: 4,4-difluoro-6-[(triisopropylsilyl)oxy]-1-hexanol

To a mixture of the compound prepared in Reference Example 43 (730 mg) in THF (9 mL) was added LiAlH₄ (123 mg). The reaction mixture was stirred at 25° C. for 2 h. The reaction mixture was quenched by water, and 15% NaOHaq. The suspension was diluted with THF and filtered. The filter cake was washed with THF. The filtrate was concentrated in vacuo to give the title compound (640 mg), which was used in the next step without any further purification.

### Reference Example 45: [(3,3-difluoro-6-iodohexyl)oxy](triisopropyl)silane

To a solution of I₂ (785 mg) in DCM (8 mL) were added PPh₃ (811 mg), imidazole (281 mg) and the compound prepared in Reference Example 44 (640 mg). The reaction mixture was stirred at 25° C. for 16 h. The reaction mixture was diluted with DCM and filtered. The filtrate was concentrated in vacuum to give a residue. The residue was purified by silica column chromatography (0~6% EtOAc in PE) to give the title compound (360 mg).

TLC R_{f}=0.7 (PE/EtOAc=10/1).

### Reference Example 46: 2-(3,5-difluorophenyl)-4-[({4,4-difluoro-6-[(triisopropylsilyl)oxy]hexyl}oxy)methyl]-1,3-thiazole

To a mixture of [2-(3,5-difluorophenyl)-1,3-thiazol-4-yl]methanol (CAS No. 1480594-24-8, 100 mg) in DMF (2 mL) was added NaH (35 mg, 60% in oil) at 0° C. The mixture was stirred at 0° C. for 30 min and followed by addition of the compound prepared in Reference Example 45 (222 mg). The reaction mixture was stirred at 25° C. for 16 h. The reaction mixture was diluted with water and extracted with EtOAc. The combined organic layers were washed with brine, dried over anhydrous Na₂SO₄ and concentrated. The residue was purified by flash column with silica gel (0~8% EtOAc in PE) to give the title compound (110 mg).

TLC R_{f}=0.7 (PE/EtOAc=10/1).

### Example 158: (3S,4R,5R,6S)-1-(6-{[2-(3,5-difluorophenyl)-1,3-thiazol-4-yl]methoxy}-3,3-difluorohexyl)-3,4,5,6-azepanetetrol

Example 158 was synthesized according to procedures analogous to Reference Example 15, 16 and Example 69 using the compound prepared in Reference Example 46 instead of the compound prepared in Reference Example 14.

LCMS: retention time 0.732 min, (Method A); MS (ESI, Pos.): m/z 531.1 (M+Na)+;

¹H NMR (CD₃OD) δ: 7.62-7.53, 7.13-7.04, 4.67, 3.63, 3.58-3.52, 3.38, 2.81, 2.74-2.66, 2.54, 2.14-1.92, 1.87-1.75.

### Example 159 to 161

Example 159 to 161 were synthesized according to procedures analogous to Reference Example 46, 15, 16 and Example 69 using the corresponding alcohol compounds instead of [2-(3,5-difluorophenyl)-1,3-thiazol-4-yl]methanol.

### Example 159: (3S,4R,5R,6S)-1-(6-{[2-(3,4-difluorophenyl)-1,3-thiazol-4-yl]methoxy}-3,3-difluorohexyl)-3,4,5,6-azepanetetrol

LCMS: retention time 1.724 min, (Method B); MS (ESI, Pos.): m/z 509.1 (M+H)+;

¹H NMR (CD₃OD) δ: 7.96-7.86, 7.80-7.73, 7.51, 7.44-7.30, 4.65, 3.62, 3.59-3.52, 3.40-3.36, 2.81, 2.75-2.65, 2.54, 2.15-1.90, 1.85-1.75.

### Example 160: (3S,4R,5R,6S)-1-(3,3-difluoro-6-{[2-(2,3,4-trifluorophenyl)-1,3-thiazol-4-yl]methoxy}hexyl)-3,4,5,6-azepanetetrol

LCMS: retention time 1.753 min, (Method B); MS (ESI, Pos.): m/z 527.1 (M+H)+;

¹H NMR (CD₃OD) δ: 8.19-7.91, 7.64, 7.38-7.21, 4.69, 3.63, 3.58-3.52, 3.43-3.34, 2.81, 2.70, 2.59-2.50, 2.13-1.92, 1.86-1.75.

### Example 161: (3S,4R,5R,6S)-1-(3,3-difluoro-6-{[2-(4-fluorophenyl)-1,3-thiazol-4-yl]methoxy}hexyl)-3,4,5,6-azepanetetrol

LCMS: retention time 1.649 min, (Method B); MS (ESI, Pos.): m/z 491.2 (M+H)+;

¹H NMR (CD₃OD) δ: 8.03-7.95, 7.46, 7.27-7.19, 4.65, 3.62, 3.58-3.50, 3.41-3.36, 2.81, 2.74-2.66, 2.54, 2.14-1.91, 1.85-1.75.

### Reference Example 47: 4,4-difluoro-6-[(triisopropylsilyl)oxy]hexyl Benzoate

To a solution of the compound prepared in Reference Example 44 (15 g) in DCM (500 mL) was added TEA (10 mL), DMAP (590 mg), benzoyl chloride (6.7 mL) at 0-5° C. The reaction mixture was stirred at 20° C. for 1 h to give a yellow solution. The reaction mixture was poured into water and extracted with DCM. The combined organic phases were dried over Na₂SO₄, filtered and concentrated to dryness. The residue was purified by flash column with silica gel (EtOAc in PE from 0 to 10%) to afford the title compound (18 g).

¹H NMR (CDCl₃) δ: 8.03, 7.56, 7.44, 4.36, 3.89, 2.23-1.91, 1.12-0.99.

### Reference Example 48: 4,4-difluoro-6-hydroxyhexyl Benzoate

To a solution of the compound prepared in Reference Example 47 (15 g) in THF (100 mL) was added 1M TBAF in THF (54.3 mL) at 20° C. The reaction mixture was stirred at 20° C. for 0.5 h to give a yellow solution. The reaction mixture was concentrated to dryness. The residue was directly purified by flash column with silica gel (EtOAc in PE from 0 to 50%) to afford the title compound (8.5 g).

¹H NMR (CDCl₃) δ: 8.04, 7.57, 7.45, 4.37, 3.89, 2.24-1.99.

### Reference Example 49: 6-[(4-ethylbenzyl)oxy]-4,4-difluoro-1-hexanol

To a mixture of the compound prepared in Reference Example 48 (93 mg) in DMF (2 mL) was added NaH (11 mg, 60% in oil) at 0° C. The mixture was stirred at 0° C. for 30 min and followed by 1-(bromomethyl)-4-ethylbenzene (CAS No. 57825-30-6, 100 mg) and TBAI (13 mg). The reaction mixture was stirred at 25° C. for 12 hr. The reaction mixture was diluted with water and extracted with EtOAc. The combined organic phase was washed with brine, dried over anhydrous Na₂SO₄ and then filtered. The filtration was concentrated. The residue was purified by flash column with silica gel (EtOAc in PE from 0~30%) to give the title compound (60 mg).

¹H NMR (CDCl₃) δ: 7.23, 7.20-7.16, 4.47, 3.73-3.58, 2.64, 2.26-2.12, 2.03-1.91, 1.81-1.70, 1.25-1.20, 1.25-1.20.

### Example 162: (3S,4R,5R,6S)-1-{6-[(4-ethylbenzyl)oxy]-4,4-difluorohexyl}-3,4,5,6-azepanetetrol

Example 162 was synthesized according to procedures analogous to Reference Example 7 and Example 69 using the compound prepared in Reference Example 49 instead of the compound prepared in Reference Example 6.

LCMS: retention time 1.711 min, (Method B); MS (ESI, Pos.): m/z 418.2 (M+H)+;

¹H NMR (CD₃CN) δ: 7.26, 7.21, 4.44, 3.63-3.25, 2.85-2.42, 1.93-1.83, 1.67-1.54, 1.20.

### Reference Example 50: 1-{[2-(3-methoxyphenyl)-1,3-thiazol-4-yl]methoxy}-6-[(triisopropylsilyl)oxy]-2-hexanol

To a solution of (2-(3-methoxyphenyl)thiazol-4-yl)methanol (CAS No. 885280-53-5, 500 mg) in tert-butanol (5 mL) was added 15% NaOHaq. (5 mL), TBAI (728 mg) and triisopropyl(4-(oxiran-2-yl)butoxy)silane (0.92 g) at 20° C. The reaction mixture was stirred at 90° C. for 15 h to give a yellow solution. The reaction mixture was poured into water and extracted with EtOAc. The combined organic phases were washed with brine, dried over Na₂SO₄, filtered and concentrated to dryness. The residue was purified by flash column with silica gel (PE to 20% EtOAc in PE) to afford the title compound (390 mg).

TLC R_{f}=0.50 (PE/EtOAc=3/1).

### Reference Example 51: 1-{[2-(3-methoxyphenyl)-1,3-thiazol-4-yl]methoxy}-6-[(triisopropylsilyl)oxy]-2-hexanone

To a solution of the compound prepared in Reference Example 50 (200 mg) in DCM (5 mL) was added DMP (259 mg) at 20° C. The reaction was stirred at 20° C. for 2 h. The reaction mixture was poured into sat. NaHCO₃ aq. and extracted with DCM. The combined organic phases were washed with sat. NaHCO₃ aq., dried over Na₂SO₄, filtered and concentrated to dryness. The residue was purified by flash column with silica gel (PE to 15% EtOAc in PE) to afford the title compound (140 mg).

TLC R_{f}=0.60 (PE/EtOAc=3/1).

### Reference Example 52: 4-[({2,2-difluoro-6-[(triisopropylsilyl)oxy]hexyl}oxy)methyl]-2-(3-methoxyphenyl)-1,3-thiazole

To a solution of the compound prepared in Reference Example 51 (140 mg) in DCM (3 mL) was added diethylaminosulfur trifluoride (0.30 mL) at 20° C. The reaction mixture was stirred at 35° C. for 15 h to give a yellow solution. The reaction was quenched by sat. NaHCO₃ aq. and extracted with DCM. The combined organic phases were dried over Na₂SO₄, filtered and concentrated to dryness. The residue was purified by flash column with silica gel (PE to 8% EtOAc in PE) to afford the title compound (80 mg).

TLC R_{f}=0.80 (PE/EtOAc=3/1).

### Example 163: (3S,4R,5R,6S)-1-(5,5-difluoro-6-{[2-(3-methoxyphenyl)-1,3-thiazol-4-yl]methoxy}hexyl)-3,4,5,6-azepanetetrol

Example 163 was synthesized according to procedures analogous to Reference Example 15, 16 and Example 69 using the compound prepared in Reference Example 52 instead of the compound prepared in Reference Example 14.

LCMS: retention time 0.696 min, (Method A); MS (ESI, Pos.): m/z 503.1 (M+H)+;

¹H NMR (DMSO-D₆) δ: 7.68, 7.51, 7.45-7.40, 7.09-7.04, 4.71, 4.68, 4.50, 3.83, 3.78, 3.42-3.35, 3.20-3.15, 2.62, 2.41-2.32, 1.98-1.83, 1.39.

### Reference Example 53: 4-[(4-biphenylylmethyl)thio]-1-butanol

To a solution of 4-(bromomethyl)-1,1′-biphenyl (200 mg) and 4-mercapto-1-butanol (CAS No. 14970-83-3, 103 mg) in DMF (5 mL) was added K₂CO₃ (224 mg), the mixture was stirred at 25° C. for 2 h. The mixture was diluted with water, extracted with EtOAc. The combined organic layers were washed with brine, dried over Na₂SO₄ and concentrated. The residue was purified by flash column with silica gel (EtOAc in PE from 0 to 90%) to afford the title compound (200 mg).

¹H NMR (CDCl₃) δ: 7.70-7.52, 7.49-7.32, 3.83-3.73, 3.71-3.58, 2.50, 1.77-1.59.

### Example 164: (3S,4R,5R,6S)-1-{4-[(4-biphenylylmethyl)thio]butyl}-3,4,5,6-azepanetetrol

Example 164 was synthesized according to procedures analogous to Reference Example 12 and Example 70 using the compound prepared in Reference Example 53 instead of the compound prepared in Reference Example 11.

LCMS: retention time 0.784 min, (Method A); MS (ESI, Pos.): m/z 418.1 (M+H)+;

¹H NMR (CD₃OD) δ: 7.62-7.55, 7.45-7.37, 7.36-7.27, 3.75, 3.61-3.49, 3.44-3.35, 2.78, 2.56-2.44, 1.64-1.52.

### Reference Example 54: 4-(2-bromoethyl)-2-(4-fluorophenyl)-1,3-thiazole

To a solution of 2-(2-(4-fluorophenyl)thiazol-4-yl)ethan-1-ol (CAS No. 871689-11-1, 220 mg) in THF (5 mL) was added PPh₃ (550 mg) and CBr₄ (660 mg) at 0° C., the reaction was stirred at 15~20° C. for 5 h to give a yellow suspension. The reaction mixture was quenched by water and extracted with EtOAc. The combined organic phase were concentrated to dryness. The residue was purified by flash column with silica gel (EtOAc in PE=0~20%,) and concentrated to give the title compound (260 mg).

TLC R_{f}=0.70 (PE/EtOAc=3/1).

### Reference Example 55: 4-({2-[2-(4-fluorophenyl)-1,3-thiazol-4-yl]ethyl}thio)-1-butanol

To a solution of the compound prepared in Reference Example 54 (260 mg) in DMF (5 mL) was added K₂CO₃ (251 mg) and 4-mercapto-1-butanol (0.10 mL) at 20° C. The resulting mixture was stirred at 15~20° C. for 16 h. The reaction mixture was washed with water, extracted with EtOAc. The combined organic layers were concentrated and purified by flash column with silica gel (EtOAc in PE=10~30%) to give the title compound (290 mg).

TLC R_{f}=0.20 (EtOAc/PE=1/3).

### Reference Example 56: 4-({2-[2-(4-fluorophenyl)-1,3-thiazol-4-yl]ethyl}thio)butanal

To a solution of the compound prepared in Reference Example 55 (100 mg) in DMSO (1 mL) and DCM (2 mL) was added TEA (0.22 mL), Py·SO₃ (340 mg) at 0° C., the reaction was stirred at 0~20° C. for 18 h to give a white suspension. The mixture was quenched by water and extracted with EtOAc. The combined organic layer was concentrated and purified by flash column with silica gel (EtOAc in PE=0~20%) to give the title compound (30 mg).

LCMS: retention time 0.898 min, (Method A); MS (ESI, Pos.): m/z 309.9 [M+H]f.

### Example 165: (3S,4R,5R,6S)-1-[4-({2-[2-(4-fluorophenyl)-1,3-thiazol-4-yl]ethyl}thio)butyl]-3,4,5,6-azepanetetrol Hydrochloride (1:1)

Example 165 was synthesized according to procedures analogous to Example 70 using the compound prepared in Reference Example 56 instead of the compound prepared in Reference Example 12.

LCMS: retention time 0.701 min, (Method A); MS (ESI, Pos.): m/z 457 (M+H)+;

¹H NMR (D₂O) δ: 7.85, 7.46, 7.25, 4.09-3.99, 3.83-3.06, 2.94, 2.53, 1.86-1.66, 1.63-1.52.

### Reference Example 57: S—{2-[2-(3,5-difluorophenyl)-1,3-thiazol-4-yl]ethyl}ethanethioate

To a solution of PPh₃ (435 mg) in THF (2 mL) was added DIAD (0.33 mL) at 0-5° C., the reaction was stirred at 0-5° C. for 0.5 h to give a white suspension. Then ethanethioic S-acid (CAS No. 507-09-5, 0.12 mL) and 2-(2-(3,5-difluorophenyl)thiazol-4-yl)ethan-1-ol (CAS No. 1874632-93-5, 200 mg) in THF (1 mL) was added in the mixture at 0~5° C. The reaction was stirred at 0-5° C. for 2 h, then the reaction was stirred at 20° C. for 15 h. The reaction mixture was concentrated to dryness. The residue was purified by preparative TLC (PE/EtOAc=5/1, R_{f}=0.6) to afford the title compound (240 mg).

TLC R_{f}=0.60 (PE/EtOAc=5/1).

### Reference Example 58: 5-({2-[2-(3,5-difluorophenyl)-1,3-thiazol-4-yl]ethyl}thio)-1-pentanol

To a solution of the compound prepared in Reference Example 54 (100 mg) in methanol (1 mL) was added K₂CO₃ (138 mg) at 20° C. The reaction mixture was stirred at 20° C. for 1 h. Then 5-bromopentan-1-ol (CAS No. 34626-51-2. 279 mg) was added to the mixture and the mixture was stirred at 20° C. for 2 h. The reaction mixture was poured into water and extracted with EtOAc. The combined organic phases were washed with brine, dried over Na₂SO₄, filtered and concentrated to dryness. The residue was directly purified by flash column with silica gel (PE to 25% EtOAc in PE,) to afford the title compound (70 mg).

TLC R_{f}=0.30 (PE/EtOAc=3/1).

### Example 166: (3S,4R,5R,6S)-1-[5-({2-[2-(3,5-difluorophenyl)-1,3-thiazol-4-yl]ethyl}thio)pentyl]-3,4,5,6-azepanetetrol

Example 166 was synthesized according to procedures analogous to Reference Example 12 and Example 70 using the compound prepared in Reference Example 58 instead of the compound prepared in Reference Example 11.

LCMS: retention time 0.732 min, (Method A); MS (ESI, Pos.): m/z 489 (M+H)+;

¹H NMR (DMSO-D₆) δ: 7.61, 7.54, 7.35, 3.55-3.49, 3.39-3.32, 3.21-3.15, 3.04-2.98, 2.91-2.84, 2.65-2.57, 2.40-2.29, 1.55-1.42, 1.40-1.24.

### Reference Example 59: 4-({2-[2-(3,5-difluorophenyl)-1,3-thiazol-4-yl]ethyl}thio)-1-butanol

Titled compound was synthesized according to procedures analogous to Reference Example 54 and 55 using the compound prepared in Reference Example 41.

LCMS: retention time 0.926 min, (Method A); MS (ESI, Pos.): m/z 330.0 [M+H]⁺.

### Reference Example 60: 2-(3,5-difluorophenyl)-4-{2-[(4-{[dimethyl(2-methyl-2-propanyl)silyl]oxy}butyl)thio]ethyl}-1,3-thiazole

To a mixture of the compound prepared in Reference Example 59 (120 mg) in DMF (4 mL) was added tert-butylchlorodimethylsilane (60 mg) and imidazole (50 mg). The reaction was stirred at 20° C. for 16 h to give a colorless solution. The mixture was washed with water and extracted with EtOAc. The combined organic layers were dried over Na₂SO₄ and concentrated under reduced pressure. The crude product was purified by silica column (PE to 8% EtOAc in PE) to give the title compound (196 mg).

LCMS: retention time 1.264 min, (Method A); MS (ESI, Pos.): m/z 444.1 [M+H]⁺.

### Reference Example 61: 2-(3,5-difluorophenyl)-4-{2-[(4-{[dimethyl(2-methyl-2-propanyl)silyl]oxy}butyl)sulfonyl]ethyl}-1,3-thiazole

To a solution of the compound prepared in Reference Example 60 (140 mg) in DCM (1.5 mL) was added mCPBA (60 mg). The mixture was stirred at 20° C. for 16 h to give a colorless solution. Then mCPBA (60 mg) was added and the mixture was stirred at 20° C. for 3 h to give a white suspension. The mixture was washed with water and extracted with EtOAc. The combined organic layers were dried over Na₂SO₄ and concentrated. The residue was purified by silica column (PE to 24% EtOAc in PE) to give the title compound (104 mg).

LCMS: retention time 1.116 min, (Method A); MS (ESI, Pos.): m/z 476.2 [M+H]⁺.

### Reference Example 62: 4-({2-[2-(3,5-difluorophenyl)-1,3-thiazol-4-yl]ethyl}sulfonyl)-1-butanol

To a mixture of the compound prepared in Reference Example 61 (104 mg) in THF (2 mL) was added 1M TBAF in THF (0.44 mL). The reaction was stirred at 20° C. for 16 h to give a yellow mixture. The mixture was concentrated in vacuum. The residue was purified by silica gel column (DCM to 6% MeOH in DCM) to give the title compound (60 mg).

LCMS: retention time 0.812 min, (Method A); MS (ESI, Pos.): m/z 362.0 [M+H]⁺.

### Example 167: (3S,4R,5R,6S)-1-[4-({2-[2-(3,5-difluorophenyl)-1,3-thiazol-4-yl]ethyl}sulfonyl)butyl]-3,4,5,6-azepanetetrol

Example 167 was synthesized according to procedures analogous to Reference Example 7 and Example 69 using the compound prepared in Reference Example 62 instead of the compound prepared in Reference Example 6.

LCMS: retention time 1.467 min, (Method B); MS (ESI, Pos.): m/z 507.1 (M+H)+;

¹H NMR (CD₃OD) δ: 7.63-7.58, 7.49, 7.15-7.04, 3.65-3.56, 3.47-3.35, 3.17-3.08, 2.94-2.81, 2.62, 1.92-1.84, 1.71-1.61.

### Reference Example 63: 2-[2-(4-fluorophenyl)-1,3-thiazol-4-yl]-2-propanol

To a mixture of ethyl 2-(4-fluorophenyl)thiazole-4-carboxylate (CAS No. 132089-35-1, 0.50 g) in THF (10 mL) was added MeMgBr (2.7 mL) at 0° C. The reaction was stirred at 20° C. for 16 h under N₂ to give a brown mixture. The reaction mixture was quenched by sat. NH₄Claq. and extracted with ethyl acetate. The organic phase was washed with brine, dried over anhydrous Na₂SO₄, concentrated in vacuum. The residue was purified by flash column with silica gel (PE to 40% EtOAc in PE) to give the title compound (0.45 g).

TLC R_{f}=0.50 (PE/EtOAc=3/1).

### Reference Example 64: 4-{2-[(6-bromohexyl)oxy]-2-propanyl}-2-(4-fluorophenyl)-1,3-thiazole

To a solution of the compound prepared in Reference Example 63 (150 mg) in DMF (2 mL) was added NaH (51 mg) and 1,6-dibromohexane (CAS No. 629-03-8, 0.29 mL). The mixture was stirred at 60° C. for 16 h. The reaction was quenched with saturated aqueous NH₄Cl, extracted with EtOAc. The combined organic layers were washed with water, brine, dried over Na₂SO₄, filtered and concentrated. The residue was purified by flash column with silica gel (EtOAc in PE from 5% to 10%) to give the title compound (120 mg).

¹H NMR (CDCl₃) δ: 7.90-7.86, 7.07-7.02, 3.32-3.22, 1.85-1.75, 1.55, 1.54-1.36.

### Example 168: (3S,4R,5R,6S)-1-[6-({2-[2-(4-fluorophenyl)-1,3-thiazol-4-yl]-2-propanyl}oxy)hexyl]-3,4,5,6-azepanetetrol

Example 168 was synthesized according to procedures analogous to Example 1 using the compound prepared in Reference Example 64 instead of the compound prepared in Reference Example 1.

LCMS: retention time 0.724 min, (Method A); MS (ESI, Pos.): m/z 483.1 (M+H)+;

¹H NMR (CD₃OD) δ: 8.00, 7.37, 7.22, 3.57, 3.42-3.36, 3.31-3.28, 2.78, 2.59-2.41, 1.63, 1.58-1.49, 1.48-1.40, 1.39-1.31, 1.30-1.21.

### Reference Example 65: {[7-(2-biphenylylmethoxy)-5-heptyn-1-yl]oxy}(triisopropyl)silane

Titled compound was synthesized according to procedures analogous to Reference Example 6 using ((triisopropylsilyl)oxy)hept-5-yn-7-ol (CAS No. 1802596-80-0) and 1-(bromomethyl)-2-phenylbenzene (CAS No. 19853-09-9).

TLC Rf=0.75 (PE/EtOAc=5/1).

### Example 169: (3S,4R,5R,6S)-1-[7-(2-biphenylylmethoxy)-5-heptyn-1-yl]-3,4,5,6-azepanetetrol Hydrochloride (1:1)

Titled compound was synthesized according to procedures analogous to Reference Example 15, 16 and Example 69 using the compound prepared in Reference Example 65 instead of the compound prepared in Reference Example 14.

LCMS: retention time 0.733 min, (Method A); MS (ESI, Pos.): m/z 440.2 (M+H)+;

¹H NMR (CD₃OD) δ:7.53-7.48, 7.46-7.40, 7.40-7.34, 7.31-7.25, 4.45, 4.16-4.01, 3.83, 3.60-3.56, 3.45-3.32, 3.27-3.13, 2.33-2.20, 1.95-1.73, 1.58-1.47.

### Example 170 and 171

Example 170 and 171 were synthesized according to procedures analogous to Reference Example 21, 22, 15, 16, Example 71 and 72 using [2-(3,5-difluorophenyl)-1,3-thiazol-4-yl]methanol instead of [5-(3,5-difluorophenyl)-2-methyl-1,3-oxazol-4-yl]methanol.

### Example 170: (3S,4R,5R,6S)-1-[(5R)-6-{[2-(3,5-difluorophenyl)-1,3-thiazol-4-yl]methoxy}-5-fluorohexyl]-3,4,5,6-azepanetetrol Hydrochloride (1:1) (Isomer 1)

LCMS: retention time 0.792 min, (Method A); MS (ESI, Pos.): m/z 491.3 (M+H)+;

¹H NMR (DMSO-D₆) 6:9.39, 7.76, 7.64, 7.41, 4.86-4.61, 3.96-3.84, 3.76-3.53, 3.34, 3.28-3.20, 3.16-3.03, 1.82-1.54, 1.47-1.28;

SFC Retention Time: 4.121 min (Method: A3).

### Example 171: (3S,4R,5R,6S)-1-[(5S)-6-{[2-(3,5-difluorophenyl)-1,3-thiazol-4-yl]methoxy}-5-fluorohexyl]-3,4,5,6-azepanetetrol Hydrochloride (1:1) (Isomer 2)

LCMS: retention time 0.694 min, (Method A); MS (ESI, Pos.): m/z 491.3 (M+H)+;

¹H NMR (DMSO-D₆) δ: 9.57, 7.76, 7.68-7.59, 7.41, 4.82-4.60, 4.66, 3.98-3.83, 3.75-3.56, 3.34, 3.29-2.96, 1.83-1.2;

SFC Retention Time: 4.535 min (Method: A3).

### Example 172 to 174

Example 172 to 174 were synthesized according to procedures analogous to Reference Example 1 and Example 1 using the corresponding alcohol compound instead of (4-methylphenyl)methanol.

### Example 172: (3S,4R,5R,6S)-1-{6-[(3-ethoxy-4-fluorobenzyl)oxy]hexyl}-3,4,5,6-azepanetetrol

LCMS: retention time 0.681 min, (Method A); MS (ESI, Pos.): m/z 416.1 (M+H)+;

¹H NMR (CD₃OD) δ: 7.11-7.00, 6.92-6.82, 4.44, 4.10, 3.59, 3.48, 3.45-3.31, 2.90-2.76, 2.54, 1.66-1.48, 1.45-1.24.

### Example 173: (3S,4R,5R,6S)-1-{6-[(4-chlorobenzyl)oxy]hexyl}-3,4,5,6-azepanetetrol

LCMS: retention time 0.683 min, (Method A); MS (ESI, Pos.): m/z 388.1 (M+H)+;

¹H NMR (CD₃OD) δ: 7.33, 4.47, 3.70-3.43, 3.39, 2.86-2.74, 2.61-2.43, 1.71-1.44, 1.41-1.27.

### Example 174: (3S,4R,5R,6S)-1-(6-{[5-(3,5-difluorophenyl)-2-methyl-1,3-oxazol-4-yl]methoxy}hexyl)-3,4,5,6-azepanetetrol Hydrochloride (1:1)

LCMS: retention time 0.689 min, (Method A); MS (ESI, Pos.): m/z 493.1 (M+Na)+;

¹H NMR (CD₃OD) δ: 7.44-7.28, 7.22, 4.54, 4.17-4.03, 3.84, 3.63-3.56, 3.42-3.33, 3.22-3.04, 2.54, 1.83-1.62, 1.51-1.37.

### Pharmacological Experimental Example 1: Evaluation of Human GBA1 Enzyme Inhibitory Activity

The inhibitory activity was determined using recombinant GBA enzyme and 4-methylumbelliferyl-β-D-glucopyranoside (4-MUG) as a substrate in 384-well format. The reaction was carried out in the acidic assay buffer (pH5.2 Mcllvaine buffer, 0.25% sodium taurocholate, and 0.1% Triton X-100). 5 μL of 4× test compound solution was preincubated with the 10 μL of GBA enzyme (0.1 ng/μL) at RT for 5 min. The reaction was initiated by adding 5 μL of 2 mmol/L substrate solution followed by incubation at 37° C. for 60 min. The reaction was stopped by addition of an equal volume of stop solution (1 mol/L glycine solution adjusted to pH10 using 1 mol/L NaOH) which raised the pH for optimal fluorescence intensity. The released 4-methylumbelliferone (4-MU) was measured (excitation 355 nm, emission 450 nm) on fluorescence spectrophotometer. The blank includes GBA followed by sequential addition of stop solution and substrate. The inhibitory activity (IC₅₀ value) of a test compound was calculated from the inhibition rate of 4-MU production at each compound concentration, with the average of the blank group as 100% inhibition and the average of the compound non-treated group as 0% inhibition.

From the above pharmacological experiments, it was found that the compound of the present disclosure had GBA1 inhibitory activity. For example, the IC₅₀ values of some compounds of the present disclosure are shown in Table 1 below. These data suggest that the compound of the present disclosure may be able to bind GBA1 and stabilize the GBA1 to such an extent that it is able to protect it from degradation.

### Pharmacological Experimental Example 2: Evaluation of Human GCS Enzyme Inhibitory Activity

The lysate of HL60 cells were used in this assay. HL60 cells were thawed and cultured in RPMI1640 medium with 10% FBS. The cells were passaged once before use. The cells were collected to a centrifuge tube and washed three times with PBS, then the number of the cells was counted. The M-PER (R) Mammalian Protein Extraction Reagent containing protease cocktail inhibitor (1:100) was added to the cells at 1 mL/4×10⁷ cells, and the mixture was kept on ice for 30 min while being mixed every 5 min to lysis the cells. The cell lysate was sonicated for 15 min in ice-water bath, and centrifuged at 1000 rpm for 10 min at 4° C. The supernatant was collected and the protein concentration was determined using BCA assay. The supernatant was aliquoted and stored at −80° C. as the enzyme source. Twenty-five nanoliters of 1000× test compound was dispensed to the 384-well assay plate by ECHO. The reaction cocktail (100 mmol/L Tris buffer (pH7.5) containing 10 μmol/L NBD-C6 ceramide, 25 μmol/L UDP-glucose, 35 μmol/L dioleoylphosphatidylcholine, 1 mmol/L dithiothreitol, and 1 mmol/L EGTA) was prepared and dispensed at 15 μL to each well. The reaction was initiated by adding 10 μL of cell lysate (1 mg/mL) to the well. The plate was kept 37° C. for 90 min and the reaction was terminated by adding 75 μL of stop solution (methanol:acetonitrile (6:5) containing 200 ng/mL tolbutamide as internal control). The reaction solution was vortexed and centrifuged to obtain the supernatant for further LC/MSMS analysis. The reaction product, NBD-C6-glucosylceramide, was analyzed using AB Sciex triple quadrupole mass spectrometer. The inhibitory activity (IC₅₀ value) of a test compound was calculated from the inhibition rate of NBD-C6-glucosylceramide production at each compound concentration, with the average of the blank group as 100% inhibition and the average of the compound non-treated group as 0% inhibition.

From the above pharmacological experiments, it was found that the compound of the present disclosure had potent GCS inhibitory activity. For example, the IC₅₀ values of some compounds of the present disclosure are shown in Table 1 below.

### Pharmacological Experimental Example 3: Evaluation of Human GBA2 Enzyme Inhibitory Activity

The human GBA2 plasmid was prepared as follows. The complete open reading frames for GBA2 (human NM_020944.2) was amplified from human tissue, using specific primers containing restriction sites and an extended Kozak sequence in front of the start codon. PCR products was subsequently cloned in the pGEM-T vector, and their sequence was verified. The appropriate fragment was subcloned in pCDNA-3.1 using the added restriction sites. HEK293T cells were transiently transfected with the human GBA2 plasmid using Lipo3000 reagents. The transfected cells were homogenized in 4 volumes of ice-cold 25 mM potassium phosphate (pH5.5) followed by sonication for 3×1 min and centrifuged for 1 h at 100,000×g. The supernatant was discarded and the pellet was resuspended and centrifuged again. The membrane pellet was resuspended in ice-cold 25 mM potassium phosphate (pH5.5) which was used as crude enzyme in the following enzymatic assay.

The inhibitory activity was determined using the GBA2-rich membrane suspensions prepared as above and 4-MUG as a substrate in 384-well format. The reaction was carried out in the assay buffer (pH5.2 Mcilvaine buffer, 0.25% sodium taurocholate, and 0.1% Triton X-100). 10 g of enzyme suspension was preincubated at 4° C. for 30 min with 1 mmol/L Conduritol B Epoxide (CBE) to inhibit GBA1. The reaction was initiated by adding 0.5 mmol/L (final concentration) substrate solution, followed by incubation at 37° C. for 45 min. The reaction was stopped by addition of stop solution (400 mmol/L glycine solution adjusted to pH10.6 using 1 mol/L NaOH). The released 4-methylumbelliferone was measured (excitation 362 nm, emission 450 nm) on fluorescence spectrophotometer. The inhibitory activity (IC₅₀ value) of a test compound was calculated from the inhibition rate of 4-MU production at each compound concentration, with the average of the blank group as 100% inhibition and the average of the compound non-treated group as 0% inhibition.

From the above pharmacological experiments, it was found that the compound of the present disclosure did not have potent GBA2 inhibitory activity. The compound of the present disclosure was found to be a compound with less concern for the accumulation of glycolipid by GBA2 inhibition. For example, the IC₅₀ values of some compounds of the present disclosure are shown in Table 1 below.

TABLE 1

| | GBA1 | GCS | GBA2 |
|---|---|---|---|
| | inhibition | inhibition | inhibition |
| Example | IC₅₀ (μM) | IC₅₀ (μM) | IC₅₀ (μM) |
| 1 | 5.1 | 2.0 | >10 |
| 21 | 2.4 | 0.06 | >10 |
| 72 | 1.2 | 0.19 | >10 |
| 74 | 1.7 | 0.03 | >10 |
| 101 | 1.1 | 0.17 | >10 |
| 107 | 1.2 | 0.13 | >10 |
| 135 | 3.2 | 0.67 | >10 |
| 139 | 5.1 | 0.22 | >10 |

Using the compound described in UV6.2 (Comparative compound A), or UV6.8 (Comparative compound B) in Patent Document 1, or the Compound 3 (Comparative compound C) described in Patent Document 2 as comparative compounds, the inhibitory activities of GBA1, GCS, and GBA2 were measured. The IC₅₀ values of comparative compounds are shown in Table 1-1 below.

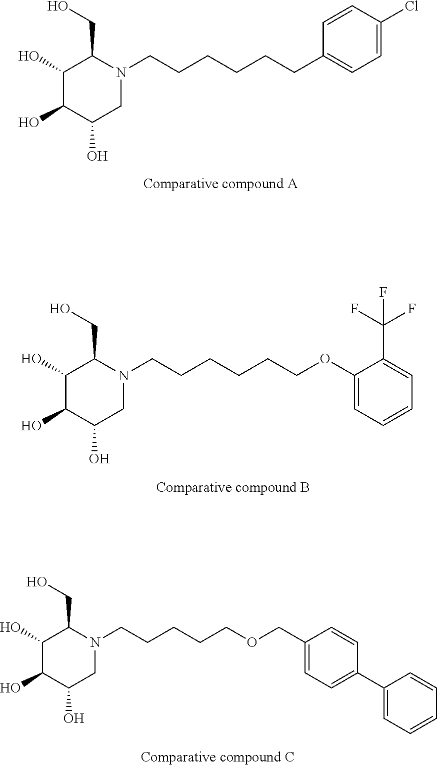 [SMILES: OC[C@@H]1[C@@H](O)[C@H](O)[C@@H](O)CN1CCCCCCc1ccc(Cl)cc1 ; OC[C@@H]1[C@@H](O)[C@H](O)[C@@H](O)CN1CCCCCCOc1ccccc1C(F)(F)F ; OC[C@@H]1[C@@H](O)[C@H](O)[C@@H](O)CN1CCCCCOCc1ccc(-c2ccccc2)cc1]

TABLE 1-1

| | GBA1 | GCS | GBA2 |
|---|---|---|---|
| Comparative | inhibition | inhibition | inhibition |
| compound | IC₅₀ (μM) | IC₅₀ (μM) | IC₅₀ (μM) |
| A | 0.08 | 0.91 | 0.001 |
| B | 0.04 | 1.20 | 0.002 |
| C | 0.28 | 0.01 | 0.001 |

From the above results, it was found that the compound of the present disclosure has less GBA2 inhibitory activity than comparative compounds.

### Pharmacological Experimental Example 4: Evaluation of Human GBA1 Cellular Activity

The Gaucher's cell line with β-glucocerebrosidase mutation of N307S (Coriell Institute, Cat. GM00372) was cultured and expanded using EMEM with 15% FBS. The cell was passaged at least twice and could be used until the 20th passages. The cells were seeded at the density of 1.25×10⁴ cells/well in 96-well plate. After cell culturing overnight, 50 μL of cell medium was discarded, and 50 μL of the diluted compound was added (with 0.3% DMSO). The cells were treated with compound for 6 days. On day 6, the cells were washed once with HBSS and incubated in HBSS for 3 h followed by lysing using 30 μL assay buffer (pH5.2 Mcllvaine buffer supplemented with 0.25% sodium taurocholate, 0.5% Triton X-100 and protease cocktail inhibitor) for 5 min at 4° C. on shaker. For GBA enzymatic assay, 15 μL of cell lysate was incubated with 5 μL of 4-MUG (6 mmol/L) for 60 min in 384 well format followed by adding 20 μL of stop solution (1 mmol/L glycine solution adjusted to pH10 using 1 mol/L NaOH). The reaction product of 4-MU was measured (excitation 355 nm, emission 450 nm) on fluorescence spectrophotometer. For the protein quantification, 4 μL of cell lysate was mixed with 36 μL of Pierce BCA reagents for 30 min. The protein was detected according to the manufacturer's instructions. Enzyme activity in cell lysate was normalized to protein concentration. The blank included substrate and assay buffer followed by addition of stop solution. Enzyme activity in cells treated with DMSO was used as a negative control, and results were represented as the percent change in enzyme activity in cells treated with each compound. For example, the percent change in enzyme activity in cells are shown in Table 2 below.

From the above pharmacological experiments, it was found that the compound of the present disclosure had potent GBA1 chaperone activity.

TABLE 2

| | Percent change in enzyme activity at test concentration | | | | |
|---|---|---|---|---|---|
| Example | 0.12 μM | 0.37 μM | 1.1 μM | 3.3 μM | 10 μM |
| 1 | 155 | 166 | 200 | 221 | 230 |
| 72 | 160 | 214 | 248 | 259 | 284 |
| 74 | 96 | 150 | 185 | 193 | 216 |
| 100 | 142 | 147 | 145 | 163 | 169 |
| 101 | 157 | 157 | 209 | 219 | 227 |
| 107 | 109 | 110 | 141 | 157 | 199 |
| 135 | 102 | 108 | 127 | 153 | 191 |
| 139 | 115 | 119 | 154 | 157 | 204 |

### Pharmacological Experimental Example 5: Evaluation of Sphingolipids Lowering Activity

The Gaucher's cell line with β-glucocerebrosidase mutation of N307S (Coriell Institute, Cat. GM00372) will be cultured and expanded using EMEM with 15% FBS. The cell will be passaged at least twice once thawed. The cells were seeded at cell density of 1×10⁵ cells/dish in 6 cm dish. After cell culturing overnight, the cell medium will be discarded and 4 mL of test compound solution will be added (with 0.1% DMSO). The cells will be treated with compound for 6 days. On day 6, the cells will be washed twice with PBS. Then, 1 mL of 20 mmol/L acetic acid in ethanol will be added to the dish and the cells will be collected using Cell scraper. This step will be repeated to obtain 2 mL of cell extracts. Ten microliters of internal standard (β-GlcCer d18:1/17:0 (200 ng/mL), β-GalSPB d18:1 (d₅) (200 ng/mL), and β-LacCer d18:1/17:0 (200 ng/mL), Cer d18:1/17:0 (200 ng/mL) will be added to it and the mixture will be vortexed, followed by addition of 1 mL of chloroform. The mixture will be sonicated for 10 min on ice and vortexed again, then centrifugated at 4° C. for 3 min at 2,130×g. The supernatant will be collected and dried up by centrifugal concentrator. The dried sample will be dissolved by 0.2 mL of chloroform/Methanol (2:1, v/v) and the level of sphingolipids will be measured by SFC/MS/MS.

### Pharmacological Experimental Example 6: Evaluation of Cognitive Impairment in Mice

Evaluating the pharmacological activity of a test compound include improvement of cognitive impairment in mice. For example, improvement of cognitive impairment by a compound may be evaluated by using the following method.

Mice (16 to 23-month old male C57BL/6 mice (Charles River Laboratories Japan, Inc.)) are used. A test compound and its vehicle are orally administered once daily for 2 weeks. After that, the cognitive function will be assessed by the novel object recognition (NOR) test using a plastic box (460 mm*300 mm*450 mm) with a video camera as the experimental apparatus. The evaluation is performed in a dark room and the light intensity is adjusted to approximately 20 lux near the experimental apparatus. To acclimatize animals to the evaluation environment, the mice are moved to the evaluation room at least 1 hour prior to the start of the trial. The study will be conducted over 3 days. For acclimation to the experimental apparatus on Day 1, the mice are placed in a cage and allowed to move freely for 10 minutes. This step is performed twice at 4 h intervals. In the acquisition trial on Day 2, two identical objects (familiar objects) are placed on the cage and performed in the same way for 10 minutes. The two objects are placed apart on a line parallel to the long side of one cage. The distance between the object and the inner wall of the two adjacent surfaces is 10 cm. In the test trial on Day 3, one of the objects used in the acquisition trial is replaced by a novel object with a different color and shape, and the animals behave in the same manner for 10 minutes. The test trials start 24 hours (Acceptable range: 23 to 25 hours) after the acquisition trials. From the video images recorded in the test trials, the exploration time for each object is measured. From the exploration time of familiar and novel objects, the total exploration time and the novel object recognition rate are calculated by the following equation.

Total exploration time (seconds)=Exploration Time for familiar object+Exploration time for novel object

Recognition index (%)=100*(Exploration time for novel object/Total exploration time)

### Formulation Examples

The following components are mixed by an ordinary method and compressed to obtain about 10,000 tablets containing 10 mg of the active ingredient in one tablet.

| | |
|---|---|
| (3S,4R,5R,6S)-1-{6-[(4-methylbenzyl)oxy]hexyl}-3,4,5,6- | 100 g |
| azepanetetrol | |
| Carboxymethylcellulose calcium (disintegrant) | 20 g |
| Magnesium stearate (lubricant) | 10 g |
| Microcrystalline cellulose | 870 g |

### INDUSTRIAL APPLICABILITY

The compound of the present disclosure has GBA1 and GCS dual modulatory activity, and thus a drug containing the compound of the present disclosure as an active ingredient is useful as an agent for preventing and/or treating a disease associated with GBA1 and/or GCS.

1. A compound of formula (I) or a pharmaceutically acceptable salt thereof: 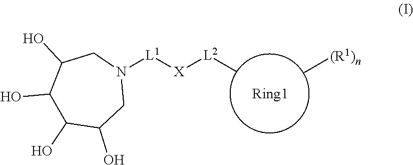
wherein
L¹ represents (1) a C4-7 alkylene, (2) a C4-7 alkenylene, or (3) a C4-7 alkynylene,
L¹ may be substituted with one to five R^{L1}s,
R^{L1} represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, or (10) a hydroxyl group,
a plurality of R^{L1}s may be the same or different,
L² represents (1) a C1-3 alkylene, (2) a C2-3 alkenylene or (3) a C2-3 alkynylene,
L² may be substituted with one to four R^{L2}s,
R^{L2} represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, or (10) a hydroxyl group,
a plurality of R^{L2}s may be the same or different,
X represents (1) a bond, (2) —O—, (3) —S—, (4) —SO—, or (5) —SO₂—,
Ring1 represents (1) a benzene ring (2) a naphthalene ring or (3) a 5- to 10-membered heterocyclyl,
R¹ represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, (10) a C5-7 carbocyclyl, (11) a 5- to 7-membered heterocyclyl, (12) —O—(C5-7 carbocyclyl), or (13) —O-(5- to 7-membered heterocyclyl),
a plurality of R¹s may be the same or different,
the groups (2) to (13) in R¹ may be substituted with one to five R¹⁻¹s,
R¹⁻¹ represents (1) a halogen atom, (2) a C1-6 alkyl group, (3) a C2-6 alkenyl group, (4) a C2-6 alkynyl group, (5) a C1-6 alkoxy group, (6) a C1-6 haloalkyl group, (7) a C2-6 haloalkenyl group, (8) a C2-6 haloalkynyl group, (9) a C1-6 haloalkoxy group, (10) a C1-6 alkyl sulfonyl group, (11) a cyano group, or (12) a phenyl group,
a plurality of R¹⁻¹s may be the same or different,
the symbol: [symbol]
represents that a substituent is bonded to the α-configuration, β-configuration or a mixture of α- and β-configurations at an appropriate ratio, and
n represents an integer of 0 to 5.

2. The compound according to claim 1 or a pharmaceutically acceptable salt thereof, wherein L¹ represents a hexylene group.

3. The compound according to claim 1 or a pharmaceutically acceptable salt thereof, wherein X represents —O—.

4. The compound according to claim 1 or a pharmaceutically acceptable salt thereof, wherein L² represents a methylene group.

5. The compound according to claim 1 or a pharmaceutically acceptable salt thereof, wherein Ring1 represents a ring structure selected from the group consisting of the following ring structures; 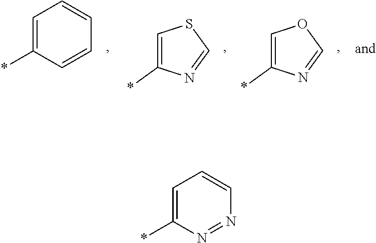 [SMILES: *c1ccccc1 ; *c1cscn1 ; *c1cocn1 ; *c1cccnn1]
wherein the * mark represents a bonding position with L².

6. The compound according to claim 1 or a pharmaceutically acceptable salt thereof, wherein R^{L1} represents (1) a halogen atom, or (2) a C1-6 alkyl group.

7. The compound according to claim 1 or a pharmaceutically acceptable salt thereof, wherein the compound of formula (I) is a compound of formula (I-1): 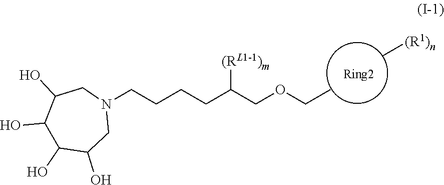
wherein R^{L1-1} represents (1) a halogen atom, or (2) a C1-6 alkyl group,
a plurality of R^{L1-1}s may be the same or different,
m represents an integer of 1 to 2,
Ring2 represents a ring structure selected from the group consisting of the following ring structures; 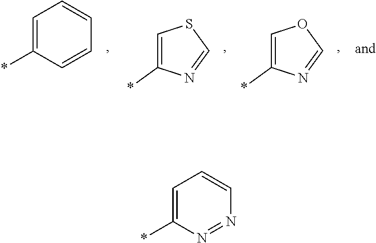 [SMILES: *c1ccccc1 ; *c1cscn1 ; *c1cocn1 ; *c1cccnn1]
wherein the * mark represents a bonding position with methylene, and
other symbols represent the same meaning as the symbols described in claim 1.

8. The compound according to claim 1 or a pharmaceutically acceptable salt thereof, wherein the compound of formula (I) is a compound selected from the group consisting of:
(1) (3S,4R,5R,6S)-1-{6-[(4-methylbenzyl)oxy]hexyl}-3,4,5,6-azepanetetrol,
(2) (3S,4R,5R,6S)-1-(6-{[2-(3,5-difluorophenyl)-1,3-thiazol-4-yl]methoxy}hexyl)-3,4,5,6-azepanetetrol,
(3) (3S,4R,5R,6S)-1-{(5R)-6-[(3-ethoxy-4-fluorobenzyl)oxy]-5-fluorohexyl}-3,4,5,6-azepanetetrol,
(4) (3S,4R,5R,6S)-1-{(5S)-6-[(3-ethoxy-4-fluorobenzyl)oxy]-5-fluorohexyl}-3,4,5,6-azepanetetrol,
(5) (3S,4R,5R,6S)-1-[(5R)-5-fluoro-6-{[2-(4-fluorophenyl)-1,3-thiazol-4-yl]methoxy}hexyl]-3,4,5,6-azepanetetrol,
(6) (3S,4R,5R,6S)-1-[(5S)-5-fluoro-6-{[2-(4-fluorophenyl)-1,3-thiazol-4-yl]methoxy}hexyl]-3,4,5,6-azepanetetrol,
(7) (3S,4R,5R,6S)-1-[(5R)-6-{[5-(3,5-difluorophenyl)-2-methyl-1,3-oxazol-4-yl]methoxy}-5-fluorohexyl]-3,4,5,6-azepanetetrol,
(8) (3S,4R,5R,6S)-1-[(5S)-6-{[5-(3,5-difluorophenyl)-2-methyl-1,3-oxazol-4-yl]methoxy}-5-fluorohexyl]-3,4,5,6-azepanetetrol,
(9) (3S,4R,5R,6S)-1-{(5S)-6-[(4-ethylbenzyl)oxy]-5-fluorohexyl}-3,4,5,6-azepanetetrol,
(10) (3S,4R,5R,6S)-1-{(5R)-6-[(4-ethylbenzyl)oxy]-5-fluorohexyl}-3,4,5,6-azepanetetrol,
(11) (3S,4R,5R,6S)-1-{(5S)-6-[(4-chlorobenzyl)oxy]-5-fluorohexyl}-3,4,5,6-azepanetetrol,
(12) (3S,4R,5R,6S)-1-{(5R)-6-[(4-chlorobenzyl)oxy]-5-fluorohexyl}-3,4,5,6-azepanetetrol,
(13) (3S,4R,5R,6S)-1-{(5S)-5-fluoro-6-[(4-methylbenzyl)oxy]hexyl}-3,4,5,6-azepanetetrol,
(14) (3S,4R,5R,6S)-1-{(5R)-5-fluoro-6-[(4-methylbenzyl)oxy]hexyl}-3,4,5,6-azepanetetrol,
(15) (3S,4R,5R,6S)-1-[(5S)-6-{[6-(3,5-difluorophenyl)-3-pyridazinyl]methoxy}-5-fluorohexyl]-3,4,5,6-azepanetetrol, and
(16) (3S,4R,5R,6S)-1-[(5R)-6-{[6-(3,5-difluorophenyl)-3-pyridazinyl]methoxy}-5-fluorohexyl]-3,4,5,6-azepanetetrol.

9. A pharmaceutical composition comprising the compound according to claim 1 or a pharmaceutically acceptable salt thereof as an active ingredient, and a pharmaceutically acceptable carrier.

10. The pharmaceutical composition according to claim 9, which is a GBA1 chaperone modulator.

11. the pharmaceutical composition according to claim 9, which is a GCS inhibitor.

12. The pharmaceutical composition according to claim 9, which is a GBA1 and GCS dual modulator.

13. The pharmaceutical composition according to claim 1, which is an agent for treating and/or preventing a disease associated with GBA1 and/or GCS.

14. The pharmaceutical composition according to claim 13, wherein the disease associated with GBA1 and/or GCS is selected from the group consisting of Parkinson's disease, Lewy body disease, Gaucher disease, Multiple system atrophy, Fabry disease, Niemann-Pick disease type C, lysosomal storage diseases, Dementia, epilepsy, bipolar disorder, schizophrenia, anxiety disorder, major depression disorder, Sandhoffs disease, Tay-Sachs disease, multiple sclerosis, Huntington's disease, Alzheimer's disease, amyotrophic lateral sclerosis, frontotemporal lobar degeneration, cognitive impairment, learning disabilities, gait disorders, sleeping disorder, neurological disorders, CNS disorders, polycystic kidney disease, type 2 diabetes, cancer, open angle glaucoma, endometriosis, multiple myeloma, virus infection, inflammation, systemic lupus erythematosus, hepatitis, Guillain-Barre Syndrome, diabetic nephropathy, and skin disorder.

15. The pharmaceutical composition according to claim 13, wherein the disease associated with GBA1 and/or GCS is selected from the group consisting of Parkinson's disease, Lewy body disease, and Gaucher disease.

16. The pharmaceutical composition according to claim 9, wherein the pharmaceutical composition is administered in combination with one or more agent(s) selected from the group consisting of levodopa, carbidopa, pramipexole, ropinirole, rotigotine, apomorphine, zonisamide, droxidopa, cabergoline, pergolide, tolcapone, entacapone, selegiline, rasagiline, safinamide, istradefylline, foslevodopa/foscarbidopa, benserazide, bromocriptine, talipexole, amantadine, trihexyphenidyl, biperiden, donepezil hydrochloride, imiglucerase, velaglucerase alfa, eliglustat, alglucerase, and miglustat.

17. (canceled)

18. A method for preventing and/or treating a disease associated with GBA1 and/or GCS, the method comprising administering (a) the compound according to claim 1 or a pharmaceutically acceptable salt thereof, or (b) a pharmaceutical composition comprising the compound according to claim 1 or a pharmaceutically acceptable salt thereof as an active ingredient, and a pharmaceutically acceptable carrier, to a patient in need of prevention and/or treatment of a disease associated with GBA1 and/or GCS.

19. (canceled)

20. (canceled)

21. The method according to claim 18, wherein the disease associated with GBA1 and/or GCS is selected from the group consisting of Parkinson's disease, Lewy body disease, Gaucher disease, Multiple system atrophy, Fabry disease, Niemann-Pick disease type C, lysosomal storage diseases, Dementia, epilepsy, bipolar disorder, schizophrenia, anxiety disorder, major depression disorder, Sandhoffs disease, Tay-Sachs disease, multiple sclerosis, Huntington's disease, Alzheimer's disease, amyotrophic lateral sclerosis, frontotemporal lobar degeneration, cognitive impairment, learning disabilities, gait disorders, sleeping disorder, neurological disorders, CNS disorders, polycystic kidney disease, type 2 diabetes, cancer, open angle glaucoma, endometriosis, multiple myeloma, virus infection, inflammation, systemic lupus erythematosus, hepatitis, Guillain-Barre Syndrome, diabetic nephropathy, and skin disorder.

22. The method according to claim 18, wherein the disease associated with GBA1 and/or GCS is selected from the group consisting of Parkinson's disease, Lewy body disease, and Gaucher disease.

23. The method according to claim 18, which further comprises administering one or more agent(s) selected from the group consisting of levodopa, carbidopa, pramipexole, ropinirole, rotigotine, apomorphine, zonisamide, droxidopa, cabergoline, pergolide, tolcapone, entacapone, selegiline, rasagiline, safinamide, istradefylline, foslevodopa/foscarbidopa, benserazide, bromocriptine, talipexole, amantadine, trihexyphenidyl, biperiden, donepezil hydrochloride, imiglucerase, velaglucerase alfa, eliglustat, alglucerase, and miglustat.

24. The method according to claim 23,
wherein the compound or a pharmaceutically acceptable thereof or the pharmaceutical composition and the one or more agent(s) are in a form of a blended drug or separate preparations; and/or
wherein the compound or a pharmaceutically acceptable thereof or the pharmaceutical composition and the one or more agent(s) are administered simultaneously or sequentially.
